SEQUENCE LISTING (151 sequences)

SEQ ID NO: 1 - skipped

SEQ ID NO: 2 - protein, 8 aa, synthetic construct
  SITE 3: note = D-Alanine
  SITE 5: note = D-Phenylalanine
RCAHFRWC

SEQ ID NO: 3 - protein, 6 aa, synthetic construct
  SITE 1: note = D-Alanine
  SITE 3: note = D-Phenylalanine
AHFRWC

SEQ ID NO: 4 - protein, 6 aa, synthetic construct
  SITE 1: note = D-Alanine
  MOD_RES 2: note = N-methyl Histidine
  SITE 3: note = D-Phenylalanine
  MOD_RES 5: note = N-methyl Tryptophan
AHFRWC

SEQ ID NO: 5 - protein, 7 aa, synthetic construct
  SITE 4: note = D-Phenylalanine
CEHFRWC

SEQ ID NO: 6 - protein, 7 aa, synthetic construct
  SITE 4: note = D-Phenylalanine
CGHFRWC

SEQ ID NO: 7 - protein, 7 aa, synthetic construct
  SITE 2: note = D-Alanine
  SITE 4: note = D-Phenylalanine
CAHFRWC

SEQ ID NO: 8 - protein, 8 aa, synthetic construct
  SITE 3: note = D-Alanine
  SITE 5: note = D-Phenylalanine
RCARFRWC

SEQ ID NO: 9 - protein, 8 aa, synthetic construct
  SITE 3: note = D-Alanine
RCAHFRWC

SEQ ID NO: 10 - protein, 8 aa, synthetic construct
  SITE 2: note = Nle
  SITE 5: note = D-Phenylalanine
RXCHFRWC

SEQ ID NO: 11 - protein, 8 aa, synthetic construct
  SITE 2: note = Nle
  SITE 5: note = D-Phenylalanine
RXCRFRWC

SEQ ID NO: 12 - protein, 8 aa, synthetic construct
  SITE 3: note = D-Alanine
  SITE 5: note = D-Phenylalanine
  SITE 7: note = D-Phenylalanine
RCARFRFC

SEQ ID NO: 13 - protein, 8 aa, synthetic construct
  SITE 3: note = D-Alanine
  SITE 5: note = D-Phenylalanine
  SITE 7: note = D-Tryptophan
RCAHFRWC

SEQ ID NO: 14 - protein, 8 aa, synthetic construct
  SITE 3: note = D-Alanine
  SITE 5: note = D-Phenylalanine
  SITE 7: note = D-Tryptophan
RCARFRWC

SEQ ID NO: 15 - protein, 8 aa, synthetic construct
  SITE 3: note = D-Alanine
  SITE 5: note = D-Phenylalanine
  SITE 7: note = D-3-(2-Naphthyl)alanine
RCARFRXC

SEQ ID NO: 16 - protein, 8 aa, synthetic construct
  SITE 3: note = D-Alanine
  SITE 5: note = D-Phenylalanine
  SITE 7: note = D-Phenylalanine
RCARFRFC

SEQ ID NO: 17 - protein, 8 aa, synthetic construct
  SITE 5: note = D-Phenylalanine
RCARFRWC

SEQ ID NO: 18 - protein, 8 aa, synthetic construct
  SITE 5: note = D-Phenylalanine
  SITE 7: note = D-Tryptophan
RCARFRWC

SEQ ID NO: 19 - protein, 8 aa, synthetic construct
  MOD_RES 3: note = N-methylalanine
  SITE 5: note = D-Phenylalanine
  SITE 7: note = D-Tryptophan
RCARFRWC

SEQ ID NO: 20 - protein, 8 aa, synthetic construct
  SITE 3: note = Aib
  SITE 5: note = D-Phenylalanine
  SITE 6: note = Citrulline
RCXRFXWC

SEQ ID NO: 21 - protein, 8 aa, synthetic construct
  SITE 3: note = D-Alanine
  MOD_RES 5: note = alpha-Methyl-D-phenylalanine
RCARFRWC

SEQ ID NO: 22 - protein, 8 aa, synthetic construct
  SITE 3: note = D-Alanine
  SITE 5: note = D-Phenylalanine
  SITE 6: note = Citrulline
  SITE 7: note = D-Tryptophan
RCARFXWC

SEQ ID NO: 23 - protein, 8 aa, synthetic construct
  SITE 3: note = D-Alanine
  SITE 5: note = D-Phenylalanine
  MOD_RES 6: note = N-methylarginine
  SITE 7: note = D-Tryptophan
RCARFRWC

SEQ ID NO: 24 - protein, 8 aa, synthetic construct
  SITE 3: note = D-Alanine
  SITE 4: note = Citrulline
  LIPID 5: note = D-Phenylalanine
RCAXFRWC

SEQ ID NO: 25 - protein, 8 aa, synthetic construct
  SITE 3: note = D-Alanine
  SITE 4: note = Citrulline
  SITE 5: note = D-Phenylalanine
  SITE 7: note = D-Tryptophan
RCAXFRWC

SEQ ID NO: 26 - protein, 8 aa, synthetic construct
  SITE 4: note = Citrulline
  SITE 5: note = D-Phenylalanine
RCAXFRWC

SEQ ID NO: 27 - protein, 8 aa, synthetic construct
  SITE 4: note = Citrulline
  SITE 5: note = D-Phenylalanine
  SITE 7: note = D-Tryptophan
RCAXFRWC

SEQ ID NO: 28 - protein, 8 aa, synthetic construct
  SITE 3: note = 2-Aminoisobutyric acid
  SITE 4: note = Citrulline
  SITE 5: note = D-Phenylalanine
RCXXFRWC

SEQ ID NO: 29 - protein, 8 aa, synthetic construct
  SITE 3: note = 2-Aminoisobutyric acid
  SITE 4: note = Citrulline
  SITE 5: note = D-Phenylalanine
  SITE 7: note = D-Tryptophan
RCXXFRWC

SEQ ID NO: 30 - protein, 8 aa, synthetic construct
  SITE 3: note = 2-Aminoisobutyric acid
  SITE 5: note = D-Phenylalanine
RCXHFRWC

SEQ ID NO: 31 - protein, 8 aa, synthetic construct
  SITE 3: note = 2-Aminoisobutyric acid
  SITE 5: note = D-Phenylalanine
RCXRFRWC

SEQ ID NO: 32 - protein, 8 aa, synthetic construct
  SITE 5: note = D-Phenylalanine
RCGRFRWC

SEQ ID NO: 33 - protein, 8 aa, synthetic construct
  SITE 4: note = Citrulline
  SITE 5: note = D-Phenylalanine
RCGXFRWC

SEQ ID NO: 34 - protein, 8 aa, synthetic construct
  SITE 3: note = D-2-Aminobutyric acid
  SITE 5: note = D-Phenylalanine
RCXHFRWC

SEQ ID NO: 35 - protein, 8 aa, synthetic construct
  SITE 3: note = 2-Aminoisobutyric acid
  SITE 5: note = D-Phenylalanine
  SITE 7: note = D-Tryptophan
RCXHFRWC

SEQ ID NO: 36 - protein, 8 aa, synthetic construct
  SITE 3: note = 2-Aminoisobutyric acid
  SITE 5: note = D-Tryosine
  SITE 7: note = D-Tryptophan
RCXRYRWC

SEQ ID NO: 37 - protein, 8 aa, synthetic construct
  SITE 3: note = 2-Aminoisobutyric acid
  SITE 5: note = D-Tyrosine
RCXRYRFC

SEQ ID NO: 38 - protein, 8 aa, synthetic construct
  SITE 3: note = 2-Aminoisobutyric acid
  SITE 4: note = Citrulline
  SITE 5: note = D-Tyrosine
RCXXYRFC

SEQ ID NO: 39 - protein, 8 aa, synthetic construct
  SITE 3: note = 2-Aminoisobutyric acid
  SITE 5: note = D-Phenylalanine
RCXRFRFC

SEQ ID NO: 40 - protein, 8 aa, synthetic construct
  SITE 3: note = 2-Aminoisobutyric acid
  SITE 4: note = Orn
  SITE 5: note = D-Phenylalanine
RCXXFRFC

SEQ ID NO: 41 - protein, 7 aa, synthetic construct
  SITE 3: note = 2-Aminoisobutyric acid
  DOMAIN 4: note = Homocitrulline
  SITE 5: note = D-Phenylalanine
RCXFRFC

SEQ ID NO: 42 - protein, 8 aa, synthetic construct
  SITE 3: note = 2-Aminoisobutyric acid
  SITE 4: note = 3-(2-Pyridyl)alanine
  SITE 5: note = D-Phenylalanine
RCXXFRFC

SEQ ID NO: 43 - protein, 8 aa, synthetic construct
  SITE 3: note = 2-Aminoisobutyric acid
  SITE 4: note = 3-(3-Pyridyl)alanine
  SITE 5: note = D-Phenylalanine
RCXXFRFC

SEQ ID NO: 44 - protein, 8 aa, synthetic construct
  SITE 3: note = 2-Aminoisobutyric acid
  SITE 5: note = 3-(4-Pyridyl)alanine
RCXXFRFC

SEQ ID NO: 45 - protein, 8 aa, synthetic construct
  SITE 2: note = Nle
  SITE 4: note = Citrulline
  SITE 5: note = D-Phenylalanine
RXCXFRWC

SEQ ID NO: 46 - protein, 8 aa, synthetic construct
  SITE 2: note = Nle
  SITE 4: note = Citrulline
  SITE 5: note = D-Phenylalanine
  SITE 7: note = D-Tryptophan
RXCXFRWC

SEQ ID NO: 47 - protein, 8 aa, synthetic construct
  SITE 2: note = Nle
  SITE 4: note = Citrulline
RXCXFRWC

SEQ ID NO: 48 - protein, 8 aa, synthetic construct
  SITE 2: note = Nle
  SITE 5: note = 3-(1-Naphthyl)alanine
RXCHXRWC

SEQ ID NO: 49 - protein, 8 aa, synthetic construct
  SITE 2: note = Nle
  SITE 5: note = 3-(1-Naphthyl)alanine
RXCRXRWC

SEQ ID NO: 50 - protein, 8 aa, synthetic construct
  SITE 2: note = Nle
  SITE 5: note = D-Phenylalanine
  SITE 7: note = D-Tryptophan
RXCRFRWC

SEQ ID NO: 51 - protein, 8 aa, synthetic construct
  SITE 2: note = Nle
  SITE 4: note = Orn
  SITE 5: note = D-Phenylalanine
RXCXFRWC

SEQ ID NO: 52 - protein, 8 aa, synthetic construct
  SITE 2: note = Nle
  SITE 4: note = Homocitrulline
  SITE 5: note = D-Phenylalanine
RXCXFRWC

SEQ ID NO: 53 - protein, 8 aa, synthetic construct
  SITE 2: note = Nle
  SITE 4: note = 3-(2-Pyridyl)alanine
  SITE 5: note = D-Phenylalanine
RXCXFRWC

SEQ ID NO: 54 - protein, 8 aa, synthetic construct
  SITE 2: note = Nle
  SITE 4: note = 3-(3-Pyridyl)alanine
  SITE 5: note = D-Phenylalanine
RXCXFRWC

SEQ ID NO: 55 - protein, 8 aa, synthetic construct
  SITE 2: note = Nle
  SITE 4: note = 3-(4-Pyridyl)alanine
  SITE 5: note = D-Phenylalanine
RXCXFRWC

SEQ ID NO: 56 - protein, 8 aa, synthetic construct
  SITE 2: note = Nle
  SITE 5: note = D-Phenylalanine
RXCRFRFC

SEQ ID NO: 57 - protein, 8 aa, synthetic construct
  SITE 2: note = Nle
  SITE 5: note = D-Phenylalanine
RXCRFRFC

SEQ ID NO: 58 - protein, 8 aa, synthetic construct
  SITE 2: note = Nle
  SITE 5: note = D-Tyrosine
RXCRYRFC

SEQ ID NO: 59 - protein, 8 aa, synthetic construct
  SITE 2: note = Nle
  SITE 5: note = D-Phenylalanine
RXCHFRFC

SEQ ID NO: 60 - protein, 8 aa, synthetic construct
  SITE 2: note = Nle
  SITE 4: note = Citrulline
  SITE 5: note = D-Phenylalanine
RXCXFRFC

SEQ ID NO: 61 - protein, 7 aa, synthetic construct
  SITE 2: note = D-Alanine
  SITE 5: note = D-Phenylalanine
CARFRWC

SEQ ID NO: 62 - protein, 7 aa, synthetic construct
  SITE 2: note = D-Alanine
  SITE 3: note = Citrulline
  SITE 4: note = D-Phenylalanine
CAXFRWC

SEQ ID NO: 63 - protein, 7 aa, synthetic construct
  SITE 4: note = D-Phenylalanine
CGRFRWC

SEQ ID NO: 64 - protein, 7 aa, synthetic construct
  SITE 3: note = Citrulline
  SITE 4: note = D-Phenylalanine
CGXFRWC

SEQ ID NO: 65 - protein, 7 aa, synthetic construct
  SITE 2: note = 2-Aminoisobutyric acid
  SITE 4: note = D-Phenylalanine
CXHFRWC

SEQ ID NO: 66 - protein, 7 aa, synthetic construct
  SITE 2: note = 2-Aminoisobutyric acid
  SITE 4: note = D-Phenylalanine
CXRFRWC

SEQ ID NO: 67 - protein, 7 aa, synthetic construct
  SITE 2: note = 2-Aminoisobutyric acid
  SITE 3: note = Citrulline
  SITE 4: note = D-Phenylalanine
CXXFRWC

SEQ ID NO: 68 - protein, 7 aa, synthetic construct
  SITE 2: note = 2-Aminoisobutyric acid
  SITE 4: note = D-Phenylalanine
CXHFRFC

SEQ ID NO: 69 - protein, 7 aa, synthetic construct
  SITE 2: note = 2-Aminoisobutyric acid
  SITE 4: note = D-Phenylalanine
CXRFRFC

SEQ ID NO: 70 - protein, 7 aa, synthetic construct
  SITE 2: note = 2-Aminoisobutyric acid
  SITE 3: note = Citrulline
  SITE 4: note = D-Phenylalanine
CXXFRFC

SEQ ID NO: 71 - protein, 7 aa, synthetic construct
  SITE 4: note = D-Phenylalanine
CGHFRFC

SEQ ID NO: 72 - protein, 7 aa, synthetic construct
  SITE 4: note = D-Phenylalanine
CGRFRFC

SEQ ID NO: 73 - protein, 7 aa, synthetic construct
  SITE 4: note = D-Phenylalanine
CGXFRFC

SEQ ID NO: 74 - protein, 6 aa, synthetic construct
  SITE 1: note = D-Alanine
  SITE 3: note = D-Phenylalanine
ARFRWC

SEQ ID NO: 75 - protein, 6 aa, synthetic construct
  SITE 1: note = D-Alanine
  SITE 2: note = Citrulline
  SITE 3: note = D-Phenylalanine
AXFRWC

SEQ ID NO: 76 - protein, 6 aa, synthetic construct
  SITE 3: note = D-Phenylalanine
GHFRWC

SEQ ID NO: 77 - protein, 6 aa, synthetic construct
  SITE 3: note = D-Phenylalanine
GRFRWC

SEQ ID NO: 78 - protein, 6 aa, synthetic construct
  SITE 3: note = D-Phenylalanine
  SITE 2: note = Citrulline
GXFRWC

SEQ ID NO: 79 - protein, 6 aa, synthetic construct
  SITE 1: note = 2-Aminoisobutyric acid
  SITE 3: note = D-Phenylalanine
XHFRWC

SEQ ID NO: 80 - protein, 6 aa, synthetic construct
  SITE 1: note = 2-Aminoisobutyric acid
  SITE 3: note = D-Phenylalanine
XRFRWC

SEQ ID NO: 81 - protein, 6 aa, synthetic construct
  SITE 1: note = 2-Aminoisobutyric acid
  SITE 2: note = Citrulline
  SITE 3: note = D-Phenylalanine
XXFRWC

SEQ ID NO: 82 - protein, 6 aa, synthetic construct
  SITE 1: note = 2-Aminoisobutyric acid
  SITE 3: note = D-Phenylalanine
XHFRFC

SEQ ID NO: 83 - protein, 6 aa, synthetic construct
  SITE 1: note = 2-Aminoisobutyric acid
  SITE 3: note = D-Phenylalanine
XRFRFC

SEQ ID NO: 84 - protein, 6 aa, synthetic construct
  SITE 1: note = 2-Aminoisobutyric acid
  SITE 2: note = Citrulline
  SITE 3: note = D-Phenylalanine
XXFRFC

SEQ ID NO: 85 - protein, 6 aa, synthetic construct
  SITE 3: note = D-Phenylalanine
GHFRFC

SEQ ID NO: 86 - protein, 6 aa, synthetic construct
  SITE 3: note = D-Phenylalanine
GRFRFC

SEQ ID NO: 87 - protein, 6 aa, synthetic construct
  SITE 2: note = Citrulline
  SITE 3: note = D-Phenylalanine
GXFRFC

SEQ ID NO: 88 - protein, 6 aa, synthetic construct
  SITE 1: note = D-Alanine
  SITE 3: note = D-Phenylalanine
AHFRFC

SEQ ID NO: 89 - protein, 6 aa, synthetic construct
  SITE 1: note = D-Alanine
  SITE 3: note = D-Phenylalanine
ARFRFC

SEQ ID NO: 90 - protein, 6 aa, synthetic construct
  SITE 1: note = D-Alanine
  SITE 2: note = Citrulline
  SITE 3: note = D-Phenylalanine
AXFRFC

SEQ ID NO: 91 - protein, 10 aa, synthetic construct
  SITE 1: note = Nle
  SITE 4: note = 3-(2-Naphthyl)alanine
  SITE 6: note = D-Tryptophan
XCHXRWCRFG

SEQ ID NO: 92 - protein, 8 aa, synthetic construct
  SITE 3: note = D-Alanine
  SITE 4: note = 4-bromo-L-phenylalanine
RCARFRWC

SEQ ID NO: 93 - protein, 8 aa, synthetic construct
  SITE 3: note = D-Alanine
  SITE 5: note = 4-bromo-D-phenylalanine
  SITE 7: note = D-Tryptophan
RCARFRWC

SEQ ID NO: 94 - protein, 8 aa, synthetic construct
  SITE 3: note = D-Alanine
  SITE 5: note = 4-bromo-L-phenylalanine
  SITE 7: note = D-Tryptophan
RCARFRWC

SEQ ID NO: 95 - protein, 11 aa, synthetic construct
  SITE 3: note = D-Alanine
  SITE 5: note = 4-bromo-D-phenylalanine
  SITE 7: note = D-Tryptophan
RCARFRWCKPV

SEQ ID NO: 96 - protein, 10 aa, synthetic construct
  SITE 3: note = D-Alanine
  SITE 5: note = 4-bromo-D-phenylalanine
  SITE 7: note = D-Tryptophan
RCARFRWCKP

SEQ ID NO: 97 - protein, 9 aa, synthetic construct
  SITE 3: note = D-Alanine
  SITE 5: note = 4-bromo-D-phenylalanine
  SITE 7: note = D-Tryptophan
RCARFRWCK

SEQ ID NO: 98 - protein, 11 aa, synthetic construct
  SITE 3: note = D-Alanine
  SITE 5: note = 4-bromo-D-phenylalanine
  SITE 7: note = D-Tryptophan
RCARFRWCRFG

SEQ ID NO: 99 - protein, 10 aa, synthetic construct
  SITE 3: note = D-Alanine
  SITE 5: note = 4-bromo-D-phenylalanine
  SITE 7: note = D-Tryptophan
RCARFRWCRF

SEQ ID NO: 100 - protein, 10 aa, synthetic construct
  SITE 3: note = D-Alanine
  SITE 5: note = 4-bromo-D-phenylalanine
  SITE 7: note = D-Tryptophan
  SITE 10: note = D-Phenylalanine
RCARFRWCRF

SEQ ID NO: 101 - protein, 8 aa, synthetic construct
  SITE 3: note = D-Alanine
  SITE 5: note = 4-bromo-D-phenylalanine
  SITE 7: note = D-Tryptophan
RCARFRWC

SEQ ID NO: 102 - protein, 8 aa, synthetic construct
  SITE 2: note = Nle
  SITE 5: note = 4-bromo-D-phenylalanine
RXCHFRWC

SEQ ID NO: 103 - protein, 8 aa, synthetic construct
  SITE 2: note = Nle
  SITE 5: note = 4-bromo-D-phenylalanine
RXCRFRWC

SEQ ID NO: 104 - protein, 8 aa, synthetic construct
  SITE 2: note = Nle
  SITE 5: note = 4-bromo-D-phenylalanine
  SITE 7: note = D-Tryptophan
RXCHFRWC

SEQ ID NO: 105 - protein, 8 aa, synthetic construct
  SITE 2: note = Nle
  SITE 5: note = 4-bromo-D-phenylalanine
  SITE 7: note = D-Tryptophan
RXCRFRWC

SEQ ID NO: 106 - protein, 8 aa, synthetic construct
  SITE 2: note = Nle
  SITE 5: note = 4-iodo-D-phenylalanine
  SITE 7: note = D-Tryptophan
RXCHFRWC

SEQ ID NO: 107 - protein, 7 aa, synthetic construct
  SITE 1: note = Nle
  SITE 4: note = 4-iodo-D-phenylalanine
  SITE 6: note = D-Tryptophan
XCHFRWC

SEQ ID NO: 108 - protein, 8 aa, synthetic construct
  SITE 3: note = D-Alanine
  SITE 5: note = D-Tyrosine
  SITE 7: note = D-Tryptophan
RCARFRWC

SEQ ID NO: 109 - protein, 8 aa, synthetic construct
  SITE 3: note = D-Alanine
  SITE 5: note = D-Tyrosine
  SITE 7: note = D-Tryptophan
RCARYRWC

SEQ ID NO: 110 - protein, 8 aa, synthetic construct
  SITE 3: note = D-Alanine
  SITE 5: note = O-methyl-D-tyrosine
  SITE 7: note = D-Tryptophan
RCARYRWC

SEQ ID NO: 111 - protein, 8 aa, synthetic construct
  SITE 3: note = D-Alanine
  SITE 5: note = Homophenylalanine
  SITE 7: note = D-Tryptophan
RCARXRWC

SEQ ID NO: 112 - protein, 8 aa, synthetic construct
  SITE 3: note = D-Alanine
  MOD_RES 5: note = alpha-methyl-D-Phenylalanine
  SITE 7: note = D-Tryptophan
RCARFRWC

SEQ ID NO: 113 - protein, 8 aa, synthetic construct
  SITE 3: note = D-Alanine
  SITE 5: note = 4-tert-butyl-D-phenylalanine
  SITE 7: note = D-Tryptophan
RCARFRWC

SEQ ID NO: 114 - protein, 10 aa, synthetic construct
  SITE 3: note = D-Alanine
  SITE 5: note = N-Fmoc-3-(4-biphenyl)-D-alanine
  SITE 7: note = D-Tryptophan
RCARBIPRWC

SEQ ID NO: 115 - protein, 8 aa, synthetic construct
  SITE 3: note = D-Alanine
  SITE 5: note = D-Tryptophan
  SITE 7: note = D-Tryptophan
RCARWRWC

SEQ ID NO: 116 - protein, 8 aa, synthetic construct
  SITE 3: note = D-Alanine
  SITE 5: note = N-alpha-Fmoc-4-(Boc-amino)-D-phenylalanine
  SITE 7: note = D-Tryptophan
RCARFRWC

SEQ ID NO: 117 - protein, 8 aa, synthetic construct
  SITE 3: note = 2-Aminoisobutyric acid
  SITE 5: note = 4-bromo-D-phenylalanine
  SITE 7: note = D-Tryptophan
RCXRFRWC

SEQ ID NO: 118 - protein, 10 aa, synthetic construct
  SITE 3: note = D-2-Aminobutyric acid
  SITE 5: note = 4-bromo-D-phenylalanine
  SITE 7: note = D-Tryptophan
RCABURFRWC

SEQ ID NO: 119 - protein, 8 aa, synthetic construct
  SITE 3: note = D-Alanine
  MOD_RES 4: note = N-methylarginine
  SITE 5: note = 4-bromo-D-phenylalanine
  SITE 7: note = D-Tryptophan
RCARFRWC

SEQ ID NO: 120 - protein, 8 aa, synthetic construct
  SITE 3: note = D-Alanine
  SITE 5: note = 4-bromo-D-phenylalanine
  SITE 7: note = D-Tryptophan
RCAPFRWC

SEQ ID NO: 121 - protein, 8 aa, synthetic construct
  SITE 3: note = D-Alanine
  SITE 4: note = D-Arginine
  SITE 5: note = 4-bromo-D-phenylalanine
  SITE 7: note = D-Tryptophan
RCARFRWC

SEQ ID NO: 122 - protein, 8 aa, synthetic construct
  SITE 3: note = D-Alanine
  SITE 5: note = 4-bromo-L-phenylalanine
  MOD_RES 6: note = N-methylarginine
  SITE 7: note = D-Tryptophan
RCARFRWC

SEQ ID NO: 123 - protein, 8 aa, synthetic construct
  SITE 3: note = D-Alanine
  SITE 5: note = 4-bromo-D-phenylalanine
  SITE 6: note = D-Arginine
  SITE 7: note = D-Tryptophan
RCARFRWC

SEQ ID NO: 124 - protein, 8 aa, synthetic construct
  SITE 3: note = D-Alanine
  SITE 5: note = 4-bromo-D-phenylalanine
  SITE 6: note = Citrulline
  SITE 7: note = D-Tryptophan
RCARFXWC

SEQ ID NO: 125 - protein, 8 aa, synthetic construct
  SITE 3: note = D-Alanine
  SITE 5: note = 4-bromo-D-phenylalanine
  SITE 7: note = D-3-(2-Naphthyl)alanine
RCARFRXC

SEQ ID NO: 126 - protein, 8 aa, synthetic construct
  SITE 3: note = D-Alanine
  SITE 5: note = 4-bromo-D-phenylalanine
  SITE 7: note = D-Phenylalanine
RCARFRFC

SEQ ID NO: 127 - protein, 8 aa, synthetic construct
  SITE 3: note = D-Alanine
  SITE 5: note = 4-bromo-D-phenylalanine
  SITE 7: note = D-2,3,4,5-Tetrahydroisoquinoline-3-carboxylic acid
RCARFRXC

SEQ ID NO: 128 - protein, 8 aa, synthetic construct
  SITE 3: note = D-Alanine
  SITE 5: note = 4-bromo-D-phenylalanine
RCARFRFC

SEQ ID NO: 129 - protein, 8 aa, synthetic construct
  SITE 3: note = D-Alanine
  SITE 5: note = 4-bromo-D-phenylalanine
  SITE 7: note = 2,3,4,5-Tetrahydroisoquinoline-3-carboxylic acid
RCARFRXC

SEQ ID NO: 130 - protein, 8 aa, synthetic construct
  SITE 3: note = D-Alanine
  SITE 5: note = 4-bromo-D-phenylalanine
  SITE 7: note = D-Alanine
RCARFRAC

SEQ ID NO: 131 - protein, 8 aa, synthetic construct
  SITE 3: note = 2-Aminoisobutyric acid
  SITE 5: note = D-Phenylalanine
  SITE 6: note = Citrulline
  SITE 7: note = D-Tryptophan
RCXRFXWC

SEQ ID NO: 132 - protein, 8 aa, synthetic construct
  SITE 3: note = D-Alanine
  MOD_RES 5: note = N-methyl-D-Phenylalanine
  SITE 7: note = D-Tryptophan
RCARFRWC

SEQ ID NO: 133 - protein, 8 aa, synthetic construct
  SITE 3: note = D-Alanine
  SITE 5: note = D-2,3,4,5-Tetrahydroisoquinoline-3-carboxylic acid
  SITE 7: note = D-Tryptophan
RCARXRWC

SEQ ID NO: 134 - protein, 8 aa, synthetic construct
  SITE 3: note = D-Alanine
  SITE 5: note = D-Tryptophan
RCARWRWC

SEQ ID NO: 135 - protein, 8 aa, synthetic construct
  SITE 3: note = D-Alanine
  SITE 5: note = D-Tryptophan
  SITE 7: note = D-Tryptophan
RCARWRWC

SEQ ID NO: 136 - protein, 8 aa, synthetic construct
  SITE 3: note = D-Alanine
  SITE 5: note = D-Homophenylalanine
  SITE 7: note = D-Tryptophan
RCARXRWC

SEQ ID NO: 137 - protein, 8 aa, synthetic construct
  SITE 3: note = D-Alanine
  MOD_RES 5: note = alpha-methyl-D-Phenylalanine
  SITE 7: note = D-Tryptophan
RCARFRWC

SEQ ID NO: 138 - protein, 8 aa, synthetic construct
  SITE 3: note = D-Alanine
  SITE 5: note = D-Diphenylalanine
  SITE 7: note = D-Tryptophan
RCARXRWC

SEQ ID NO: 139 - protein, 8 aa, synthetic construct
  SITE 3: note = D-Alanine
  SITE 5: note = D-Phenylalanine
  SITE 6: note = D-Arginine
RCARFRWC

SEQ ID NO: 140 - protein, 8 aa, synthetic construct
  SITE 3: note = D-Alanine
  SITE 5: note = D-Phenylalanine
  SITE 6: note = D-Arginine
  SITE 7: note = D-Tryptophan
RCARFRWC

SEQ ID NO: 141 - protein, 8 aa, synthetic construct
  SITE 3: note = 2-Aminoisobutyric acid
  SITE 4: note = Citrulline
  MOD_RES 5: note = O-methyl-D-tyrosine
  SITE 7: note = D-Tryptophan
RCXXYRWC

SEQ ID NO: 142 - protein, 8 aa, synthetic construct
  SITE 2: note = Nle
  SITE 5: note = 4-bromo-D-phenylalanine
RXCRFRWC

SEQ ID NO: 143 - protein, 8 aa, synthetic construct
  SITE 2: note = Nle
  SITE 5: note = 4-bromo-D-phenylalanine
  SITE 7: note = D-Tryptophan
RXCRFRWC

SEQ ID NO: 144 - protein, 8 aa, synthetic construct
  SITE 2: note = Nle
  MOD_RES 5: note = O-methyl-D-tyrosine
RXCRYRWC

SEQ ID NO: 145 - protein, 8 aa, synthetic construct
  SITE 2: note = Nle
  MOD_RES 5: note = O-methyl-D-tyrosine
  SITE 7: note = D-Tryptophan
RXCRYRWC

SEQ ID NO: 146 - protein, 7 aa, synthetic construct
  SITE 5: note = 4-bromo-D-phenylalanine
CGRFRFC

SEQ ID NO: 147 - protein, 7 aa, synthetic construct
  MOD_RES 4: note = O-methyl-D-tyrosine
CGRYRFC

SEQ ID NO: 148 - protein, 8 aa, synthetic construct
  SITE 3: note = D-Alanine
  SITE 5: note = 3-(2-Naphthyl)alanine
RCAPXRWC

SEQ ID NO: 149 - protein, 8 aa, synthetic construct
  SITE 3: note = D-Alanine
  SITE 5: note = 4-bromo-D-phenylalanine
RCAPFRWC

SEQ ID NO: 150 - protein, 8 aa, synthetic construct
  SITE 3: note = D-Alanine
  SITE 5: note = 4-bromo-D-phenylalanine
RCARFRWC

SEQ ID NO: 151 - protein, 8 aa, synthetic construct
  SITE 5: note = 4-bromo-D-phenylalanine
  SITE 3: note = D-Alanine
RCAHFRWC

(19) United States
(12) Patent Application Publication
(10) Pub. No.: US 2024/0391954 A1
(43) Pub. Date: Nov. 28, 2024

(54) MC4R AGONIST PEPTIDES

(71) Applicants: The Regents of the University of Michigan, Ann Arbor, MI (US); Courage Therapeutics, Inc., Newton, MA (US)

(72) Inventors: Savannah Y. WILLIAMS, Ann Arbor, MI (US); Luis E. GIMENEZ, Ann Arbor, MI (US); Tomi SAWYER, Newton, MA (US); Roger D. CONE, Ann Arbor, MI (US)

(21) Appl. No.: 18/686,144

(22) PCT Filed: Aug. 24, 2022
(86) PCT No.: PCT/US2022/075425

§ 371 Date: Feb. 23, 2024

Related U.S. Application Data
(60) Provisional application No. 63/236,485, filed on Aug. 24, 2021.

(51) Int. Cl. C07K 7/06; A61K 38/00; A61P 3/04

(52) U.S. Cl. CPC C07K 7/06; A61P 3/04; A61K 38/00

(57) ABSTRACT

Provided herein are melanocortin 4 receptor (MC4R) agonist peptides and methods of use thereof for the treatment and/or prevention of eating disorders (e.g., overeating), metabolic disorders (e.g., disorders resulting in positive energy imbalance), emotional/mental disorders, and/or dietary or syndromic obesity. In particular, provided herein are MC4R agonist peptides that exhibit enhanced selectivity for MC4R over other melanocortin receptors (e.g., MC1R, MC2R, MC3R, MC5R) and/or are MC3R antagonists or partial agonists. The peptides herein may exhibit enhanced in vitro potency, in vivo efficacy, pharmacokinetic properties, and/or stability compared to other known melanocortin receptor binding peptides.

### CROSS-REFERENCE TO RELATED APPLICATIONS

This application claims the benefit of U.S. Provisional Patent Application No. 63/236,485, filed on Aug. 24, 2021, which is incorporated by reference herein.

### SEQUENCE LISTING

The text of the computer readable sequence listing filed herewith, titled “39120-601_SEQUENCE_LISTING”, created Aug. 24, 2022, having a file size of 272,745 bytes, is hereby incorporated by reference in its entirety

### FIELD

Provided herein are melanocortin 4 receptor (MC4R) agonist peptides and methods of use thereof for the treatment and/or prevention of eating disorders (e.g., overeating), metabolic disorders (e.g., disorders resulting in positive energy imbalance), emotional/mental disorders, and/or dietary or syndromic obesity. In particular, provided herein are MC4R agonist peptides that exhibit enhanced selectivity for MC4R over other melanocortin receptors (e.g., MC1R, MC2R, MC3R, MC5R) and/or are MC3R antagonists or partial agonists. The peptides herein may exhibit enhanced in vitro potency, in vivo efficacy, pharmacokinetic properties, and/or stability compared to other known melanocortin receptor binding peptides.

### BACKGROUND

Obesity is a multifactorial condition involving an excessive amount of body fat. Obesity is a medical problem that increases the risk of other diseases and health problems, such as heart disease, diabetes, high blood pressure and certain cancers. A large body of research has established the critical role of hypothalamic AgRP neural circuits in stimulating feeding and intense effort has focused on identifying pharmacological targets that suppress these circuits as potential therapeutics for obesity. Therapeutics for the treatment/prevention of dietary or syndromic obesity, the upstream causes, and downstream effects are needed.

### SUMMARY

Provided herein are melanocortin 4 receptor (MC4R) agonist peptides and methods of use thereof for the treatment and/or prevention of eating disorders (e.g., overeating), metabolic disorders (e.g., disorders resulting in positive energy imbalance), emotional/mental disorders, and/or dietary or syndromic obesity. In particular, provided herein are MC4R agonist peptides that exhibit enhanced selectivity for MC4R over other melanocortin receptors (e.g., MC1R, MC2R, MC3R, MC5R) and/or are MC3R antagonists or partial agonists. The peptides herein may exhibit enhanced in vitro potency, in vivo efficacy, pharmacokinetic properties, and/or stability compared to other known melanocortin receptor binding peptides.

In some embodiments, an MC4R agonist peptide (and/or MC3R antagonist or partial agonist) herein exhibits 70% or greater in vivo efficacy (e.g., >70%, >75%, >80%, >85%, >90%, >95%, etc.).

In some embodiments, peptide herein exhibits selectivity for MC4R over other melanocortin receptors (e.g., MC1R, MC2R, MC3R, MC5R). In some embodiments, a peptide herein exhibits 10-fold or greater selectivity for MC4R over MC3R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC4R agonist peptide (and/or MC3R antagonist or partial agonist) herein exhibits 10-fold or greater selectivity for MC3R over MC1R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC4R agonist peptide (and/or MC3R antagonist or partial agonist) herein exhibits 10-fold or greater selectivity for MC3R over MC2R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC4R agonist peptide (and/or MC3R antagonist or partial agonist) herein exhibits 10-fold or greater selectivity for MC3R over MC5R (e.g., 10-fold, 15-fold, 20-fold, 25-fold, 30-fold, 40-fold, 50-fold, 60-fold, 70-fold, 80-fold, 90-fold, 100-fold, or more or ranges therebetween).

In some embodiments, peptide herein exhibits selectivity for MC3R over other melanocortin receptors (e.g., MC1R, MC2R, MC4R, MC5R). In some embodiments, a peptide herein exhibits 10-fold or greater selectivity for MC3R over MC4R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC4R agonist peptide (and/or MC3R antagonist or partial agonist) herein exhibits 10-fold or greater selectivity for MC3R over MC1R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC4R agonist peptide (and/or MC3R antagonist or partial agonist) herein exhibits 10-fold or greater selectivity for MC3R over MC2R (e.g., 10-fold, 15-fold, 20-fold, 25-fold, 30-fold, 40-fold, 50-fold, 60-fold, 70-fold, 80-fold, 90-fold, 100-fold, or more or ranges therebetween). In some embodiments, an MC4R agonist peptide (and/or MC3R antagonist or partial agonist) herein exhibits 10-fold or greater selectivity for MC3R over MC5R (e.g., 10-fold, 15-fold, 20-fold, 25-fold, 30-fold, 40-fold, 50-fold, 60-fold, 70-fold, 80-fold, 90-fold, 100-fold, or more or ranges therebetween).

In some embodiments, provided herein are methods (e.g., of treating an eating disorder (e.g., overeating), a metabolic disorder (e.g., disorders resulting in positive energy imbalance), emotional/mental disorders, and/or obesity) in a subject, the methods comprising administering a melanocortin 4 receptor (MC4R) agonist to a subject suffering from the eating disorder. In some embodiments, provided herein are methods (e.g., of treating an eating disorder (e.g., overeating), a metabolic disorder (e.g., disorders resulting in positive energy imbalance), emotional/mental disorders, and/or obesity) in a subject, the methods comprising administering a melanocortin 3 receptor (MC3R) antagonist to a subject suffering from the eating disorder. In some embodiments, provided herein are methods (e.g., of treating an eating disorder (e.g., overeating), a metabolic disorder (e.g., disorders resulting in positive energy imbalance), emotional/mental disorders, and/or obesity) in a subject, the methods comprising administering an MC3R partial agonist to a subject (e.g., a subject suffering form or at increased risk of the condition to be treated/prevented). In some embodiments, the eating disorder is characterized by overeating. In some embodiments, the eating disorder is characterized by one or more emotional/mental symptoms. In some embodiments, the eating disorder is characterized by anxiety and/or depression. In some embodiments, the eating disorder is stress-induced overeating. In some embodiments, the MC4R agonist (and/or MC3R antagonist or partial agonist) is a peptide. In some embodiments, a peptide is both an MC4R agonist and MC3R antagonist. In some embodiments, the MC4R agonist (and/or MC3R antagonist or partial agonist) is a peptide. In some embodiments, a peptide is both an MC4R agonist and MC3R partial agonist). In some embodiments, a peptide is an MC3R partial agonist which activates MC3R to a lesser extent than a full agonist and therefore reduces MC3R activation by competing with native full agonists. In some embodiments, an MC3R partial agonist activates MC3R to less than 50% of full activation (e.g., <40%, <30%, <20%, <10%, <5%, etc.). In some embodiments, the administration is repeated on a recurring basis for a period of at least 1 week (e.g., 1 week, 2 weeks, 1 month, 2 months, 4 months, 6 months, 9 months, 1 year, 2 years, 3, years, 4 years, or more). In some embodiments, the administration is repeated on a daily basis. In some embodiments, the administration is repeated on a twice-daily basis. In some embodiments, the administration is repeated on alternate days. In some embodiments, the administration is repeated on a weekly basis. In some embodiments, the administration is repeated on a recurring basis for a period of at least 1 month (e.g., 1 month, 2 months, 4 months, 6 months, 9 months, 1 year, 2 years, 3, years, 4 years, or more). In some embodiments, the administration is repeated on a recurring basis for a period of at least 1 year.

In some embodiments, provided herein are compositions comprising a peptide having 4 or fewer substitutions relative to the sequence: X-AA1-AA1B-AA2-AA3-AA4-AA5-AA6-AA7-AA8-AA9-AA10-AA11-Y (SEQ ID NO: 1); wherein X is a N-terminal cap moiety linked to the most N-terminal amino acid of the peptide and is acetyl, chloro acetyl, or absent; wherein AA1 is Arg or absent; wherein AA1B is Nle or absent; wherein AA2 is Cys or absent; wherein AA3 is D-Ala, Glu, D-Glu, D-Gly, D-Aib, Gly, Ala, NMe-Ala, Aib, Abu, D-Abu, or absent; wherein AA4 is Arg, D-Arg, NMe-Arg, NMe-D-Arg, Cit, D-Cit, His, D-His, Nme-His, NMe-D-His, Pro, D-Orn, Orn, Homo-Arg, Homo-Cit, Homo-D-Cit, Pal(2′), Pal(3′), Pal(4′), D-Pal(2′), D-Pal(3′), 4-guanidyl-Dab, 4-guanidyl-D-Dab, 3-guanidyl-Dap, 3-guanidyl-D-Dap, 5-carbamoyl-Dab, 5-carbamoyl-D-Dab, 3-carbamoyl-Dap, 3-carbamoyl-D-Dap, or D-Pal(4′); wherein AA5 is Phe, D-Phe, D-Phe(4-Br), D-Phe(4-I), D-Phe(4-F), D-Phe(4-tBu), Phe(4-Br), Phe(4-F), Nal(2′), D-Nal(2′), Nal(1′), D-Tyr, Tyr(4-OMe), or D-Tyr(4-OMe), D-Hph, D-Bip, D-Tic, D-Dip, D-Trp, aMe-D-Phe, D-Phe(4-NH-Ac), NMe-D-Phe, Phe(4-tBu), Trp, Hph, Bip, Tic, Dip, D-Nal(1′), aMe-Phe, Phe(4-NH-Ac), NMe-Phe, Tyr, D-Nal(1′), Phe(4-I), D-Phe(4-I), Phe(4-tBu)], D-Phe(4-guanidyl), or Phe(4-guanidyl); wherein AA6 is Arg, NMe-Arg, D-Arg, Cit, NMe-D-Arg, or D-Cit; wherein AA7 is Trp, D-Trp, NMe-Trp, Phe, D-Phe, D-Ala D-Nal(2′), D-Tic, NMe-D-Trp, Ala, Nal(2′), or Tic; wherein AA8 is Cys; wherein AA9 is Lys, Arg, or absent; wherein AA10 is Pro, Phe, D-Phe, or absent; wherein AA11 is Val, Gly, or absent; wherein Y is a C-terminal cap linked to the most C-terminal amino acid of the peptide and is NH₂ or absent; wherein AA1 is linked to AA2 if AA1B is absent; wherein AA9 is present if AA10 is present; wherein AA9 and AA10 are present if AA11 is present; wherein the peptide does not consist of Arg-Cys-(D-Ala)-His-(D-Phe)-Arg-Trp-Cys (SEQ ID NO: 2).

In some embodiments, provided herein are compositions comprising a peptide having 100% sequence similarity to the sequence: X-AA1-AA1B-AA2-AA3-AA4-AA5-AA6-AA7-AA8-AA9-AA10-AA11-Y (SEQ ID NO: 1); wherein X is a N-terminal cap moiety linked to the most N-terminal amino acid of the peptide and is acetyl, chloro acetyl, or absent; wherein AA1 is Arg or absent; wherein AA1B is Nle or absent; wherein AA2 is Cys or absent; wherein AA3 is D-Ala, Glu, D-Glu, D-Gly, D-Aib, Gly, Ala, NMe-Ala, Aib, Abu, D-Abu, or absent; wherein AA4 is Arg, D-Arg, NMe-Arg, NMe-D-Arg, Cit, D-Cit, His, D-His, Nme-His, NMe-D-His, Pro, D-Orn, Orn, Homo-Arg, Homo-Cit, Homo-D-Cit, Pal(2′), Pal(3′), Pal(4′), D-Pal(2′), D-Pal(3′), 4-guanidyl-Dab, 4-guanidyl-D-Dab, 3-guanidyl-Dap, 3-guanidyl-D-Dap, 5-carbamoyl-Dab, 5-carbamoyl-D-Dab, 3-carbamoyl-Dap, 3-carbamoyl-D-Dap, or D-Pal(4′); wherein AA5 is Phe, D-Phe, D-Phe(4-Br), D-Phe(4-I), D-Phe(4-F), D-Phe(4-tBu), Phe(4-Br), Phe(4-F), Nal(2′), D-Nal(2′), Nal(1′), D-Tyr, Tyr(4-OMe), or D-Tyr(4-OMe), D-Hph, D-Bip, D-Tic, D-Dip, D-Trp, aMe-D-Phe, D-Phe(4-NH-Ac), NMe-D-Phe, Phe(4-tBu), Trp, Hph, Bip, Tic, Dip, D-Nal(1′), aMe-Phe, Phe(4-NH-Ac), NMe-Phe, Tyr, D-Nal(1′), Phe(4-I), D-Phe(4-I), Phe(4-tBu)], D-Phe(4-guanidyl), or Phe(4-guanidyl); wherein AA6 is Arg, NMe-Arg, D-Arg, Cit, NMe-D-Arg, or D-Cit; wherein AA7 is Trp, D-Trp, NMe-Trp, Phe, D-Phe, D-Ala D-Nal(2′), D-Tic, NMe-D-Trp, Ala, Nal(2′), or Tic; wherein AA8 is Cys; wherein AA9 is Lys, Arg, or absent; wherein AA10 is Pro, Phe, D-Phe, or absent; wherein AA11 is Val, Gly, or absent; wherein Y is a C-terminal cap linked to the most C-terminal amino acid of the peptide and is NH₂ or absent; wherein AA1 is linked to AA2 if AA1B is absent; wherein AA9 is present if AA10 is present; wherein AA9 and AA10) are present if AA11 is present; wherein the peptide does not consist of Arg-Cys-(D-Ala)-His-(D-Phe)-Arg-Trp-Cys (SEQ ID NO: 2).

In some embodiments, provided herein are compositions comprising a peptide having the sequence; X-AA1-AA1B-AA2-AA3-AA4-AA5-AA6-AA7-AA8-AA9-AA10-AA11-Y (SEQ ID NO: 1); wherein X is a N-terminal cap moiety linked to the most N-terminal amino acid of the peptide and is acetyl, chloro acetyl, or absent; wherein AA1 is Arg or absent; wherein AA1B is Nle or absent; wherein AA2 is Cys or absent; wherein AA3 is D-Ala, Glu, D-Glu, D-Gly, D-Aib, Gly, Ala, NMe-Ala, Aib, Abu, D-Abu, or absent; wherein AA4 is Arg, D-Arg, NMe-Arg, NMe-D-Arg, Cit, D-Cit, His, D-His, Nme-His, NMe-D-His, Pro, D-Orn, Orn, Homo-Arg, Homo-Cit, Homo-D-Cit, Pal(2′), Pal(3′), Pal(4′), D-Pal(2′), D-Pal(3), 4-guanidyl-Dab, 4-guanidyl-D-Dab, 3-guanidyl-Dap, 3-guanidyl-D-Dap, 5-carbamoyl-Dab, 5-carbamoyl-D-Dab, 3-carbamoyl-Dap, 3-carbamoyl-D-Dap, or D-Pal (4); wherein AA5 is Phe, D-Phe, D-Phe(4-Br), D-Phe(4-I), D-Phe(4-F), D-Phe(4-tBu), Phe(4-Br), Phe(4-F), Nal(2′), D-Nal(2′), Nal(1′), D-Tyr, Tyr(4-OMe), or D-Tyr(4-OMe), D-Hph, D-Bip, D-Tic, D-Dip, D-Trp, aMe-D-Phe, D-Phe(4-NH-Ac), NMe-D-Phe, Phe(4-tBu), Trp, Hph, Bip, Tic, Dip, D-Nal(1′), aMe-Phe, Phe(4-NH-Ac), NMe-Phe, Tyr, D-Nal(1′), Phe(4-I), D-Phe(4-I), Phe(4-tBu)], D-Phe(4-guanidyl), or Phe(4-guanidyl); wherein AA6 is Arg, NMe-Arg, D-Arg, Cit, NMe-D-Arg, or D-Cit; wherein AA7 is Trp, D-Trp, NMe-Trp, Phe, D-Phe, D-Ala D-Nal(2), D-Tic, NMe-D-Trp, Ala, Nal(2′), or Tic; wherein AA8 is Cys; wherein AA9 is Lys, Arg, or absent; wherein AA10 is Pro, Phe, D-Phe, or absent; wherein AA11 is Val, Gly, or absent; wherein Y is a C-terminal cap linked to the most C-terminal amino acid of the peptide and is NH₂ or absent; wherein AA1 is linked to AA2 if AA1B is absent; wherein AA9 is present if AA10 is present; wherein AA9 and AA10 are present if AA11 is present; wherein the peptide does not consist of Arg-Cys-(D-Ala)-His-(D-Phe)-Arg-Trp-Cys (SEQ ID NO: 2).

In some embodiments, provided herein are peptides having 1-4 substitutions or terminal deletions relative to the amino acid sequence Arg-Cys-(D-Ala)-His-(D-Phe)-Arg-Trp-Cys (SEQ ID NO: 2). In some embodiments, peptides is selected from one or SEQ ID NOS: 3-151. In some embodiments, the peptide comprises one or more non-proteinogenic amino acids or amino acid analogs. In some embodiments, any L amino acids in the peptides herein may be substituted for D amino acids. In some embodiments, any D amino acids in the peptides herein may be substituted for L amino acids. In some embodiments, the peptide is cyclic. In some embodiments, AA3 is absent and AA1b is present. In some embodiments, AA3 is present and AA1b is absent.

In some embodiments, all or a portion of the peptide is cyclic.

In some embodiments, X is chloroacetyl, AA1, AA1b, and AA2 are absent, and the chloroacetyl reacts with the Cys at AA8 to form a thioether-linked cyclic peptide. In some embodiments, such thioether-linked cyclic peptides are provided. In some embodiments, the peptide comprises 0-4 substitutions relative to an amino acid sequence selected from SEQ ID NOS: 3-4 and 74-90. In some embodiments, the peptide comprises an amino acid sequence selected from SEQ ID NOS: 3-4 and 74-90.

In some embodiments, the amino acid corresponding to AA2 of SEQ ID NO: 1 is Cys, the amino acid corresponding to AA8 of SEQ ID NO: 1 is Cys, wherein the amino acid corresponding to AA2 of SEQ ID NO: 1 is linked to the amino acid corresponding to AA8 of SEQ ID NO: 1, and wherein the peptide segment corresponding to AA2-AA8 of SEQ ID NO: 1 is cyclic. In some embodiments, such cys-cys-linked cyclic peptides are provided. In some embodiments, the peptide further comprises an amino acid corresponding to AA1 or AA1b of SEQ ID NO: 1 linked to the amino acid corresponding to AA2 of SEQ ID NO: 1 and/or an amino acid corresponding to AA9 of SEQ ID NO: 1 linked to the amino acid corresponding to AA8 of SEQ ID NO: 1. In some embodiments, the peptide comprises 0-4 substitutions relative to an amino acid sequence selected from SEQ ID NOS: 5-73 and 91-151. In some embodiments, the peptide comprises an amino acid sequence selected from SEQ ID NOS: 3-4 and 5-73 and 91-151.

In some embodiments, the peptide comprises 0-4 substitutions relative to an amino acid sequence selected from SEQ ID NOS: 3-151. In some embodiments, the peptide comprises an amino acid sequence selected from SEQ ID NOS: 3-151.

In some embodiments, a peptide here comprises one or more non-proteinogenic amino acids or amino acid analogs. In some embodiments, a peptide herein is cyclic.

In some embodiments, provided herein are methods of treating a subject for a disease, condition, or disorder comprising administering a composition comprising a peptide described herein to the subject. In some embodiments, the subject suffers from positive energy balance as the cause or result of the disease, condition, or disorder. In some embodiments, the composition (peptide) is administered to treat (or prevent) positive energy balance. In some embodiments, the subject suffers from a disease, condition, or disorder characterized by overeating. In some embodiments, the composition (peptide) is administered to treat (or prevent) overeating. In some embodiments, the subject suffers from a disease, condition, or disorder characterized by one or more emotional/mental symptoms. In some embodiments, the composition (peptide) is administered to treat (or prevent) one or more emotional/mental symptoms. In some embodiments, the disease, condition, or disorder is caused by or is the result of obesity. In some embodiments, the composition (peptide) is administered to treat (or prevent) obesity. In some embodiments, the subject suffers from (or is at increased risk of) diabetes, heart disease, hypertension, sleep apnea, depression, kidney disease, and/or arthritis. In some embodiments, the composition (peptide) is administered to treat (or prevent) diabetes, heart disease, hypertension, sleep apnea, depression, kidney disease, and/or arthritis. In some embodiments, the composition is co-administered with nutritional therapy, psychotherapy, or other pharmaceutical agents. In some embodiments, administration is repeated on a recurring basis for a period of at least 1 week. In some embodiments, administration is repeated on a daily basis. In some embodiments, administration is repeated on a recurring basis for a period of at least 1 month. In some embodiments, administration is repeated on a recurring basis for a period of at least 1 year.

In some embodiments, provided herein is the use of a composition comprising a peptide described herein in the treatment or prevention of an condition, disease, or disorder. In some embodiments, provided herein is the use of composition comprising a peptide described herein as a medicament. In some embodiments, provided herein are compositions comprising a peptide described herein for use in the manufacture of a medicament.

In some embodiments, provided herein are compositions (e.g., pharmaceutical compositions) comprising a melanocortin 4 receptor (MC4R) agonist (and/or MC3R antagonist or partial agonist) peptide. In some embodiments, a peptide of a pharmaceutical composition herein is selective for MC4R and/or MC3R over other melanocortin receptors.

In some embodiments, the peptides herein find use in the treatment and/or prevention of various conditions, such as those related to obesity and/or overeating. In some embodiments, the MC4R agonist (and/or MC3R antagonist or partial agonist) peptides herein find use in the treatment or prevention of obesity, diabetes, heart disease, hypertension, sleep apnea, depression, kidney disease, arthritis, etc. In some embodiments, the subject suffers from obesity. In some embodiments, the subject suffers from dietary obesity. In some embodiments, the subject suffers from an obesity syndrome due to melanocortin-4 receptor haploinsufficiency. In some embodiments, the subject is at risk of overeating or becoming obese. In some embodiments, the subject has recovered from obesity or an over-eating disorder and is at risk of relapsing.

In some embodiments, provided herein are methods of treating an eating disorder comprising administering a composition (e.g., pharmaceutical compositions) comprising a peptide herein to a subject suffering from the eating disorder. In some embodiments, the eating disorder is characterized by overeating. In some embodiments, the eating disorder is characterized by one or more emotional/mental symptoms. In some embodiments, the composition is co-administered with nutritional therapy, psychotherapy, weight-management routines, a weight-loss device, bariatric surgery, diet, other weight-loss therapeutics, etc.

In some embodiments, methods are provided in which administration of a composition (e.g., pharmaceutical compositions) comprising a peptide herein is repeated on a recurring basis for a period of at least 1 week. In some embodiments, the administration is repeated on a daily basis. In some embodiments, the administration is repeated on a recurring basis for a period of at least 1 month. In some embodiments, the administration is repeated on a recurring basis for a period of at least 1 year.

In some embodiments, provided herein is the use of a composition (e.g., pharmaceutical compositions) comprising a peptide herein in the treatment or prevention of an eating disorder. In some embodiments, provided herein is the use of a composition (e.g., pharmaceutical compositions) comprising a peptide herein as a medicament. In some embodiments, provided herein is the use of a composition (e.g., pharmaceutical compositions) comprising a peptide herein the manufacture of a medicament.

### BRIEF DESCRIPTION OF THE DRAWINGS

FIG. 1. Results of pharmacological assay for setmelanotide. Concentration-response curves for setmelanotide at the human MC3R (blue) and human MC4R (red) were determined by a split-luciferase cAMP sensor dynamic assay in HEK-293 cells stably expressing the hMC3R or hMC4R and the GScAMP22F CAMP sensor. Each data point represents the mean±SEM of a representative experiment with three replicates each. Data is normalized to the ECmax of α-MSH.

FIG. 2. Results of pharmacological assay for exemplary peptides herein exhibiting dual MC4R agonist, MC3R antagonist properties. Concentration-response agonist curves for CTX-2205 and CTX-1214 at the human MC3R (blue) and human MC4R (red) were determined by a split-luciferase cAMP sensor dynamic assay in HEK-293 cells stably expressing the hMC3R or hMC4R and the GScAMP22F CAMP sensor. The antagonist curves were determined in the presence of an EC80 to EC90 concentration of α-MSH. Each data point represents the mean±SEM of two experiments with three replicates each per experiment. Data is normalized to the ECmax of α-MSH.

FIG. 3. Results of plasma stability testing for control peptides. Compounds shown were incubated in mouse plasma at 37° C. at 1 μM concentrations. 40 ul aliquots were removed at the times indicated and the reactions stopped with the addition of 160 μL cold acetonitrile containing 200 ng/ml of peptide internal standard (IS). The incubation solution was centrifuged at 3500 rpm for 10 minutes to precipitate protein. LC/MS/MS analysis was used to determine the % peptide remaining.

FIG. 4A-B. (A) Pharmacological properties of exemplary MC4R agonist peptides, including those with improved efficacy in the MC4R+/− mouse (CTX-1211, CTX-1227, and CTX-1228). Concentration-response agonist curves for peptides indicated at the human MC4R (red), human MC3R (blue), human MC5R (green), and human MC1R (black) were determined by a split-luciferase cAMP sensor dynamic assay in HEK-293 cells stably expressing the hMC1R, hMC3R, hMC4R, or hMC5R and the GScAMP22F cAMP sensor. Each data point represents the mean±SEM of two experiments with three replicates each per experiment. Data is normalized to the ECmax of α-MSH. (B) Same as above, for peptides representing reduced activity at the hMC1R. Inset tables indicate EC₅₀ values for the ligand indicated for each receptor.

FIG. 5. Pharmacokinetic data for an exemplary peptide relative to setmelanotide. Peptides at 1.5 mg/mL in PBS containing 10% DMSO and 10% PEG-400 were given by IP injection (15 mg/kg) to female CD-1 mice (n=15). At the given time points (0.5 h, 2 h, 4 h, 7 h and 24 h), brain samples were taken out and frozen at −80° C. immediately for later preparation and analysis, and blood samples were collected using heparinized calibrated pipettes. Blood samples were centrifuged at 15000 rpm for 10 min. Subsequently, blood plasma was collected from the upper layer. The plasma was frozen at −80° C. for later analysis. Peptide concentrations at times indicated were determined by LC MS/MS, using non-zero standards in blank Plasma and Brain Samples.

FIG. 6. Improved inhibition of food intake in the MC4R+/− mouse with CTX-1211, CTX-1227, and CTX-1228, relative to setmelanotide. Exemplary peptides significantly improve inhibition of food intake compared to setmelanotide control. Studies utilized 7 MC4R+/− male C57BL/6J mice per condition, 8-24 weeks of age. Mice were individually housed. Animals were injected daily at 5 PM with 150 ml of saline for 3 days or until animals acclimate, as indicated by the return of 14 hr food intake to pre-treatment levels. Animals were then randomized, and injected with either vehicle or drug in vehicle (2.5 mg/kg) on the experimental day. Injections were performed at 5 pm, 1 hour before the start of dark cycle. Food consumption was then determined at 8 pm (3 hours), 7 am (14 hours) and 5 pm next day (24 hours). Bars indicate mean±SEM.

FIG. 7. Quantification of acute feeding studies. Table shows quantification of data from FIG. 6, along with data from a similar experiment using diet-induced obese animals, produced by feeding 8 week old male C57BL/6J with high fat diet for 13 weeks.

FIG. 8. Chronic low-dose infusion studies. Peptides indicated were administered at 1200 nmol/kg/day via Alzet minipump model #1002 implanted subcutaneously in the subscapular region of 42 week old male C57BL/6J MC4R+/− mice (n=6-7). Cumulative food intake measured on the days indicated. Data points indicate mean±SEM.

FIG. 9. Inhibition of food intake by ICV administration of a dual MC4R agonist/MC3R antagonist. Individually housed obese male C57BL/6J mice were injected with either vehicle or peptides indicated, at doses shown, on the experimental day. Injections were performed at 5 pm, 1 hour before the start of dark cycle. Food consumption was then determined at 3, 6, 16, or 24 hr after compound administration. Bars indicate mean±SEM. Peptides were administered intraperitoneally (left), or intracerebroventricularly (ICV, right) via cannulas implanted in the lateral ventricle.

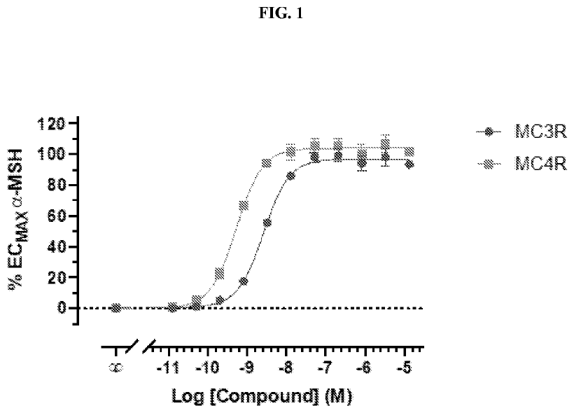

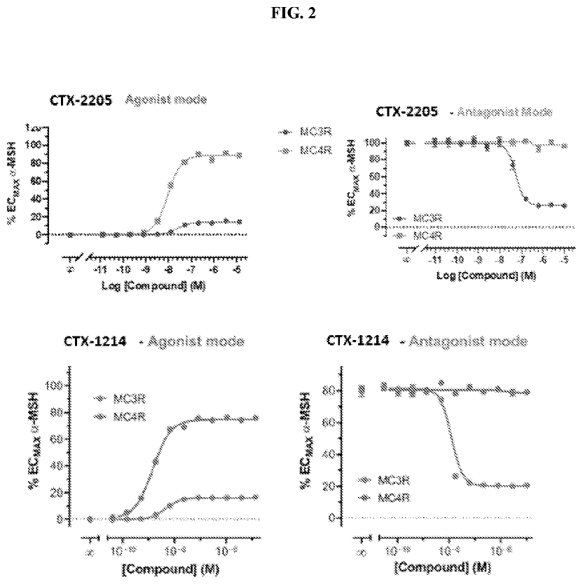

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an agonist peptide” is a reference to one or more agonist peptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of”' and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “MC4R agonist” refers to an agent (e.g., peptide, etc.) that binds to MC4R and promotes MC4R to produce its biological activity to at least the same degree as a natural ligand for MC4R (e.g., α-melanocyte stimulating hormone (α-MSH) or adrenocorticotropic hormone). In some embodiments, an MC4R agonist binds to MC4R in the same location as a natural MC3R ligand.

As used herein, the term “MC3R antagonist” refers to an agent (e.g., peptide, etc.) that binds to MC3R and inhibits MC3R from producing its biological activity. In some embodiments, an MC3R antagonist binds to MC3R in the same location as a natural MC3R ligand (e.g., melanocyte-stimulating hormone and adrenocorticotropic hormone).

As used herein, the term “MC3R partial agonist” refers to an agent (e.g., peptide, etc.) that binds to MC3R and promotes MC3R to produce its biological activity to a lesser extent than a full agonist (e.g., a natural agonist of MC3R (e.g., melanocyte-stimulating hormone and adrenocorticotropic hormone)). In some embodiments, an MC3R partial agonist binds to MC3R in the same location as a natural MC3R ligand (e.g., melanocyte-stimulating hormone and adrenocorticotropic hormone).

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “obesity” refers to a medical condition with excess body fat accumulation and people are generally defined to be obese when their body mass index (BMI; a value of body mass (kg) over body height squared (m)) is 30 or higher. Obesity is most commonly caused by energy imbalance due to excessive food intake compared to energy consumption over a long period of time (“positive energy balance”). Obesity, being a metabolic disease that affects the entire body, increases the possibility of developing of diabetes and hyperlipidemia, increases the risk of the incidence of sexual dysfunction, arthritis, and cardiovascular disease, and is associated with cancer development in some cases.

As used herein, the term “subject at risk for a disease,” for example, “a subject at risk for diabetes” or “a subject at risk for hypertension” refers to a subject with one or more risk factors (e.g., obesity, overeating, etc.) for developing the disease. Depending upon the specific disease, risk factors may include, but are not limited to, gender, age, genetic predisposition, environmental exposures, and previous incidents of diseases, lifestyle, etc.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., an MC4R agonist and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “instructions for administering said compound to a subject,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

The term “amino acid” refers to natural amino acids, unnatural amino acids, and amino acid analogs, all in their D and L stereoisomers, unless otherwise indicated, if their structures allow such stereoisomeric forms. Embodiments herein refer to various amino acid abbreviations (single-letter or three-letter abbreviations) that will be understood by those in the field. Any amino acid abbreviations not defined herein refer to their field-accepted meaning. For example, “NMe” preceding an amino acid name refers to an “N-methyl” group on the amino acid, “Nle” is “norleucine,” “Abu” is “α-Aminobutyric acid,” “Aib” is “2-Aminoisobutyric acid,” “Nal(2′) is “3-(2-Naphthyl)-L-alanine,” “tic” is “1,2,3,4-tetrahydroisoquinoline-3-carboxylic acid,” “HpH” is “homophenylalanine,” “Bip” is “N-alpha-Fmoc-beta-(4-biphenyl)-L-alanine,” “D-Phe(4tBu)” is “D-4-tert-butyl-phenylalanine,” and the single-letter or three-letter abbreviations for the common proteinogenic amino acids are provided below.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyrrolysine may also be considered proteinogenic amino acids

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxy methyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 30 amino acids or less in length. A peptide may comprise natural amino acids, non-natural amino acids, proteinogenic amino acids, non-proteinogenic amino acids, amino acid analogs, and/or modified amino acids. A peptide may be a subsequence of naturally occurring protein or a non-natural (artificial) sequence.

As used herein, the term “cyclic peptide” refers to a cyclic derivative of a peptide in which two amino acids that are not adjacent in the linear sequence are linked to form a loop in the peptide. For example, one or more additional groups suitable for cyclization may be added to facilitate cyclization of the peptide or peptide segment. A cyclic peptide may contain an intramolecular disulfide bond (e.g., —S—S—), an intramolecular amide bond between two residues, (e.g., —CONH— or —NHCO—), an intramolecular S-alkyl bond (e.g., —S—(CH₂)ₙ—CONH— or —NH—CO(CH₂)ₙ—S—, wherein n is 1-6), etc. Cyclization may be also carried out by triazine chemistry (e.g., as exemplified in Scharn, D. et al. (2001) J. Org, Chem 66; 507; incorporated by reference in its entirety). Cyclic peptides or peptide segments are denoted with the prefix “cyclo” in front of the peptide sequence and the cyclic part of the sequence within parenthesis (e.g., “Arg-cyclo(Cys-D-Ala-His-D-Phe-Arg-Trp-Cys)” wherein the two Cys residues are linked to form a cyclic peptide segment of “Cys-D-Ala-His-D-Phe-Arg-Trp-Cys”).

As used herein, the term “artificial” refers to compositions and systems that are designed or prepared synthetically, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another;
1) Alanine (A) and Glycine (G);
2) Aspartic acid (D) and Glutamic acid I;
3) Asparagine (N) and Glutamine (Q);
4) Arginine I and Lysine (K);
5) Isoleucine (I), Leucine (L), Methionine (M), and Valine (V);
6) Phenylalanine (F), Tyrosine (Y), and Tryptophan (W);
7) Serine(S) and Threonine (T); and
8) Cysteine I and Methionine (M).

Naturally occurring residues may be divided into classes based on common side chain properties, for example; polar positive (or basic) (histidine (H), lysine (K), and arginine I); polar negative (or acidic) (aspartic acid (D), glutamic acid I); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y% sequence identity (e.g., 90%) with SEQ ID NO:Z (e.g., 20 amino acids) may have up to X substitutions (e.g., 2) relative to SEQ ID NO:Z, and may therefore also be expressed as “having X (e.g., 2) or fewer substitutions relative to SEQ ID NO:Z.”

### DETAILED DESCRIPTION

Provided herein are melanocortin 4 receptor (MC4R) agonist peptides and methods of use thereof for the treatment and/or prevention of eating disorders (e.g., overeating), metabolic disorders (e.g., disorders resulting in positive energy imbalance), emotional/mental disorders, and/or dietary or syndromic obesity. In particular, provided herein are MC4R agonist peptides that exhibit enhanced selectivity for MC4R over other melanocortin receptors (e.g., MC1R, MC2R, MC3R, MC5R) and/or are MC3R antagonists or partial agonists. The peptides herein may exhibit enhanced in vitro potency, in vivo efficacy, pharmacokinetic properties, and/or stability compared to other known melanocortin receptor binding peptides.

Central regulation of feeding and body weight is primarily controlled by neural circuits located in the hypothalamus and hindbrain (Refs. 1-3; herein incorporated by reference in their entireties). The central melanocortin system, composed of a set of two neuronal cell types located in the hypothalamic arcuate nucleus, the agouti related peptide neurons (AgRP neurons) and the pro-opiomelanocortin neurons (POMC neurons), engages this hypothalamic and hindbrain circuitry to potently regulate feeding and body weight (Refs. 4-6; herein incorporated by reference in their entireties). AgRP and POMC neurons project to largely overlapping brain regions to exert opposing effects on feeding and body weight. For example, AgRP neurons synthesize and release the melanocortin receptor antagonist/inverse agonist, agouti related peptide (AgRP), GABA, and neuropeptide Y to stimulate feeding and body weight (Ref. 7; herein incorporated by reference in its entirety). In contrast, POMC neurons synthesize and release the endogenous melanocortin receptor agonist, alpha melanocyte stimulating hormone (α-MSH), in addition to fast excitatory/inhibitory neurotransmitters to suppress feeding and reduce body weight (Refs. 4, 8; herein incorporated by reference in their entireties).

Hypothalamic AgRP neurons play a potent role in stimulating feeding (Refs. 6, 9, 10; herein incorporated by reference in their entireties). Ablation of AgRP neurons in adult mice leads to starvation and death, while stimulation rapidly and robustly stimulates food intake and body weight in sated animals (Refs. 11-13; herein incorporated by reference in their entireties). In addition to stimulating feeding, AgRP neuronal activation also suppresses competing need states, such as anxiety and fear, thereby promoting food seeking behavior in response to negative energy balance (Refs. 14-15; herein incorporated by reference in their entireties). Intense effort has focused on identifying pharmacological targets which suppress AgRP neural circuits as a potential therapeutic strategy for obesity.

The melanocortin 4 receptor (MC4R), a component of the leptin-melanocortin pathway, plays a part in bodyweight regulation (Clement et al. The Lancet. 2020 December; 8(12):960-970; incorporated by reference in its entirety). Pro-opiomelanocortin (POMC)-derived peptides act on neurons expressing the Melanocortin 4 receptor (MC4R) to reduce body weight. Setmelanotide is a highly potent MC4R agonist that leads to weight loss in diet-induced obese animals and in obese individuals with complete POMC deficiency (Collet et al. Mol Metab. 2017 October; 6(10):1321-1329; incorporated by reference in its entirety):

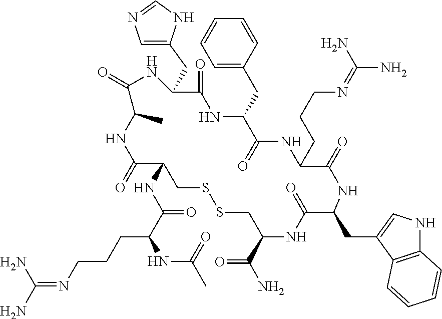 [SMILES: CC(=O)N[C@@H](CCCN=C(N)N)C(=O)N[C@H]1CSSC[C@H](C(N)=O)NC(=O)[C@H](Cc2c[nH]c3ccccc23)NC(=O)C(CCCN=C(N)N)NC(=O)[C@@H](Cc2ccccc2)NC(=O)[C@H](Cc2cnc[nH]2)NC(=O)[C@@H](C)NC1=O]

(setmelanotide). In some embodiments, the peptides provided herein are variants of setmelanotide, having sequence similarity and/or an analogous cyclization motif. In some embodiments, peptides herein comprise amino substitutions, deletions, additions, etc. relative to setmelanotide.

MC3R is a G-protein coupled receptor primarily expressed within the brain, with particular dense expression observed in the hypothalamic arcuate nucleus (Refs. 16-17; herein incorporated by reference in their entireties). MC3R is expressed in AgRP neurons and recent studies suggest that MC3R has an important role in regulating the orexigenic activity of these cells (Ref. 16; herein incorporated by reference in its entirety). For example, MC3R knockout mice show multiple deficits in conditions that activate AgRP neurons, such as impaired feeding in response to a fast or caloric restriction (Refs. 18-20; herein incorporated by reference in their entireties). MC3R acts within presynaptic AgRP terminals in the paraventricular hypothalamus (PVN), promoting GABA release onto anorexigenic PVN melanocortin 4 receptor expressing neurons (Ref. 18; herein incorporated by reference in its entirety). Further, the MC3R plays a developmental role in growth and maturation to puberty (Ref. 62; incorporated by reference in its entirety).

International Patent Application WO2020257662, which is incorporated by reference in its entirety, demonstrates and describes; that MC3R agonists stimulate feeding and increase body weight, and reduce anxiety in an AgRP neuron dependent manner; that MC3R is highly expressed in arcuate AgRP neurons, with significantly higher expression in these cells than anorexigenic POMC neurons; that MC3R agonist treatment phenocopies chemogenetic or optogenetic activation of ARC MC3R neurons, both stimulating feeding and body weight; that chemogenetic inhibition of AgRP neuron reduces feeding; and that targeting of MC3R is a therapeutic approach for combating disorders of energy metabolism.

Embodiments herein provide for the modulation (e.g., activation) of MC4R in order to achieve a desired impact on a condition of energy metabolism (e.g., obesity), eating habits (e.g., overeating, etc.), or downstream effect thereof (e.g., hypertension, heart disease, diabetes, etc.). In some embodiments, MC4R agonist peptides for activating MC4R are administered to a subject and/or co-administered with one or more additional therapeutics/therapies.

Certain embodiments herein provide for the modulation (e.g., inhibition or only partial activation) of MC3R in order to achieve a desired impact on a condition of energy metabolism (e.g., obesity), eating habits (e.g., overeating, etc.), or downstream effect thereof (e.g., hypertension, heart disease, diabetes, etc.). In some embodiments, an MC3R antagonist or partial agonist peptide for inhibiting MC3R is administered to a subject and/or co-administered with one or more additional therapeutics/therapies.

In some embodiments, provided herein are methods of treating, preventing, and/or ameliorating the symptoms of overeating, obesity, diabetes, heart disease, hypertension, sleep apnea, depression, kidney disease, arthritis, etc. by enhancing the activity of MC4R in a subject via administration of a MC4R agonist peptide. In some embodiments, provided herein are methods of treating, preventing, and/or ameliorating the symptoms of overeating, obesity, diabetes, heart disease, hypertension, sleep apnea, depression, kidney disease, arthritis, etc. by inhibiting of the activity of MC3R in a subject via administration of a MC3R antagonist or partial agonist peptide.

In some embodiments, the subject suffers from overeating, obesity (e.g., dietary obesity, syndromic obesity (e.g., melanocortin-4 receptor haploinsufficiency), etc.), diabetes, heart disease, hypertension, sleep apnea, depression, kidney disease, arthritis, etc. In some embodiments, the subject is at risk of developing obesity, diabetes, heart disease, hypertension, sleep apnea, depression, kidney disease, arthritis, etc., or having a recurrence of one of the aforementioned conditions.

In some embodiments, provided herein are MC4R agonist (and/or MC3R antagonist or partial agonist) peptides comprising sequence variants of setmelanotide (SEQ ID NO: 2). In some embodiments, provided herein are peptides of the sequence X-AA1-AA1B-AA2-AA3-AA4-AA5-AA6-AA7-AA8-AA9-AA10-AA11-Y (SEQ ID NO: 1); wherein X is a N-terminal cap moiety linked to the most N-terminal amino acid of the peptide and is acetyl, chloro acetyl, or absent; wherein AA1 is Arg or absent; wherein AA1B is Nle or absent; wherein AA2 is Cys or absent; wherein AA3 is D-Ala, Glu, D-Glu, D-Gly, D-Aib, Gly, Ala, NMe-Ala, Aib, Abu, D-Abu, or absent; wherein AA4 is Arg, D-Arg, NMe-Arg, NMe-D-Arg, Cit, D-Cit, His, D-His, Nme-His, NMe-D-His, Pro, D-Orn, Orn, Homo-Arg, Homo-Cit, Homo-D-Cit, Pal(2′), Pal(3′), Pal(4′), D-Pal(2′), D-Pal(3′), 4-guanidyl-Dab, 4-guanidyl-D-Dab, 3-guanidyl-Dap, 3-guanidyl-D-Dap, 5-carbamoyl-Dab, 5-carbamoyl-D-Dab, 3-carbamoyl-Dap, 3-carbamoyl-D-Dap, or D-Pal(4′); wherein AA5 is Phe, D-Phe, D-Phe(4-Br), D-Phe(4-I), D-Phe(4-F), D-Phe(4-tBu), Phe(4-Br), Phe(4-F), Nal(2′), D-Nal(2′), Nal(1′), D-Tyr, Tyr(4-OMe), or D-Tyr(4-OMe), D-Hph, D-Bip, D-Tic, D-Dip, D-Trp, aMe-D-Phe, D-Phe(4-NH-Ac), NMe-D-Phe, Phe(4-tBu), Trp, Hph, Bip, Tic, Dip, D-Nal(1′), aMe-Phe, Phe(4-NH-Ac), NMe-Phe, Tyr, D-Nal(1′), Phe(4-I), D-Phe(4-I), Phe(4-tBu)], D-Phe(4-guanidyl), or Phe(4-guanidyl); wherein AA6 is Arg, NMe-Arg, D-Arg, Cit, NMe-D-Arg, or D-Cit; wherein AA7 is Trp, D-Trp, NMe-Trp, Phe, D-Phe, D-Ala D-Nal(2′), D-Tic, NMe-D-Trp, Ala, Nal(2′), or Tic; wherein AA8 is Cys; wherein AA9 is Lys, Arg, or absent; wherein AA10 is Pro, Phe, D-Phe, or absent; wherein AA11 is Val, Gly, or absent; wherein Y is a C-terminal cap linked to the most C-terminal amino acid of the peptide and is NH₂ or absent; wherein AA1 is linked to AA2 if AA1B is absent; wherein AA9 is present if AA10 is present; wherein AA9 and AA10 are present if AA11 is present; wherein the peptide does not consist of Arg-Cys-(D-Ala)-His-(D-Phe)-Arg-Trp-Cys (SEQ ID NO: 2). In some embodiments, peptides comprise conservative or semiconservative substitutions relative to SEQ ID NO: 1. In some embodiments, a conservative or semi-conservative substitution may also comprise a non-proteinogenic amino acid or amino acid analog with similar characteristics.

In some embodiments, provided herein are peptides having at least 70% (e.g., >70%, >75%, >80%, >85%, >90%, >95%, 100%) conservative sequence similarity with the sequence X-AA1-AA1B-AA2-AA3-AA4-AA5-AA6-AA7-AA8-AA9-AA10-AA11-Y (SEQ ID NO: 1). In some embodiments, provided herein are peptides having at least 70% (e.g., >70%, >75%, >80%, >85%, >90%, >95%, 100%) semi-conservative sequence similarity with the sequence X-AA1-AA1B-AA2-AA3-AA4-AA5-AA6-AA7-AA8-AA9-AA10-AA11-Y (SEQ ID NO: 1). In some embodiments, provided herein are peptides having at least 70% (e.g., >70%, >75%, >80%, >85%, >90%, >95%, 100%) sequence identity with the sequence X-AA1-AA1B-AA2-AA3-AA4-AA5-AA6-AA7-AA8-AA9-AA10-AA11-Y (SEQ ID NO: 1).

In some embodiments, peptides herein have at least 1 (e.g., 1, 2, 3, 4, 5, or more) substitution or terminal deletion relative to SEQ ID NO: 2.

In some embodiments, provided herein are peptides having 4 or fewer (e.g., 4, 3, 2, 1) substitutions (e.g., conservative, semi-conservative, unconservative, etc.) relative to one or more of SEQ ID NOS: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 151.

In some embodiments, the N-terminal cap moiety linked to the most N-terminal amino acid of the peptide. In some embodiments, the N-terminal cap moiety and is an acetyl group. In some embodiments, the N-terminal cap moiety and is a chloro acetyl group. In embodiment sin which the N-terminal cap is a chloro acetyl group, the N-terminal group may react with a Cys residue within the peptide (e.g., AA8) to form a thio ether linkage, resulting in a thio-ether cyclized peptide (a cyclic peptide). In some embodiments, the N-terminal cap moiety is a pharmacokinetic (PK) modifying group. Such groups are described in Refs. 59-61 (incorporated by reference in their entireties) and include groups comprising a PK-modifying moiety (e.g., 4-iodo phenyl, C18 diacid, etc.), an amino acid linker moiety (e.g., Gly, γGlu, etc.), and a PEG moiety (e.g., methoxy PEG (e.g., mPEG-2, MPEG-3, MPEG-4, MPEG-5, mPEG-6, or more)). In some embodiments, an N-terminal cap is of the general structure:

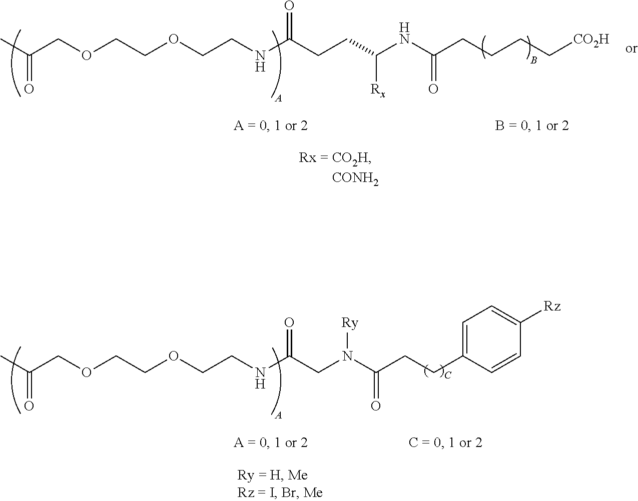

Examples of PK—modifying caps include C18 diacid-γGlu-mPEG2 (PKcap1) and Aryl(4-I)-Gly-mPEG2 (PKcap2):

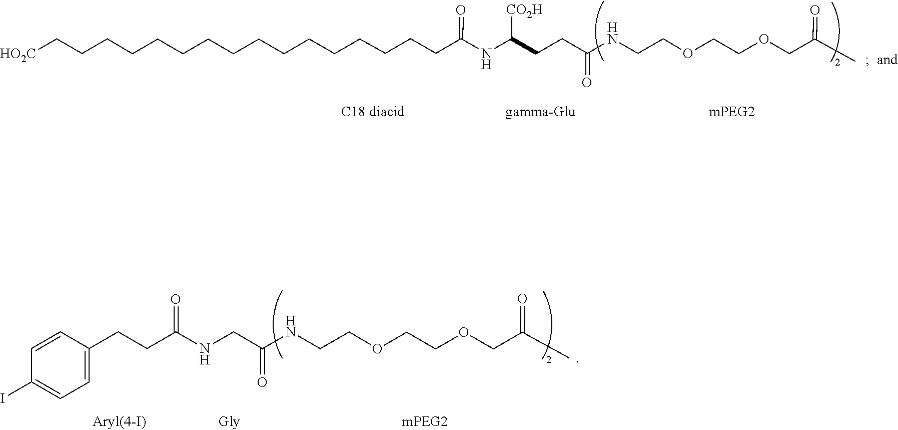

Other PK—modifying caps are within the scope herein.

In some embodiments, peptides herein comprise natural amino acids, unnatural amino acids, modified amino acids, non-proteinogenic amino acids, amino acid analogs, etc. In some embodiments, all or a portion of the peptide is cyclic. For example, certain peptides provided in Table 1 and Table 2, as depicted in those tables, comprise both a linear portion and a cyclic portion. In some embodiments, the amino acid corresponding to AA2 of SEQ ID NO: 1 is Cys, the amino acid corresponding to AA8 of SEQ ID NO: 1 is Cys, wherein the amino acid corresponding to AA2 of SEQ ID NO: 1 is linked to the amino acid corresponding to AA8 of SEQ ID NO: 1, and wherein the peptide segment corresponding to AA2-AA8 of SEQ ID NO: 1 is cyclic. In some embodiments, the peptide further comprises an amino acid corresponding to AA1 or AA1B of SEQ ID NO: 1 linked to the amino acid corresponding to AA2 of SEQ ID NO: 1 and/or an amino acid corresponding to AA9 of SEQ ID NO: 1 linked to the amino acid corresponding to AA8 of SEQ ID NO: 1. In some embodiments, the peptides of Table 1 or Table 2, or other peptides within the scope herein (e.g., corresponding to SEQ ID NO: 1) may be provided as linear peptides. In some embodiments, peptides herein (e.g., peptides corresponding SEQ ID NO: 1, peptides of Table 1 or 2, etc.) are cyclic but comprise an alternative cyclization. For example, insertion or substitution of Cys or D-Cys, or other suitable residues between or for residues in the peptides allow for cyclization of a peptide segment other than AA2-AA8. In some embodiments, a peptide herein may be cyclized by methods understood in the field and described herein. For example, a first amino acid within the sequence of the peptide may be substituted for a Cys or D-Cys, the amino acid corresponding to a second amino of the peptide may be substituted for a Cys, Orn, or D-Cys, and the amino acid corresponding to first amino acid is linked to the second amino acid, rendering the peptide segment between those amino acids of the peptide cyclicAny pairs of amino acids within the peptides herein can be used for cyclization. In some embodiments, any of AA1, AA1B, AA2, AA3, AA4, AA5, AA6, AA7, AA8, AA9, AA10, and AA11 may be amino acids capable of forming a cyclic section of peptide. For example, a five-amino-acid cyclic segment may be formed between AA1 and AA5, AA1B and AA6, AA2 and AA7, AA3 and AA8, AA4 and AA9, AA5 and AA10, or AA6 and AA11. In some embodiments, a four-amino-acid cyclic segment may be formed between AA1 and AA4, AA1B and AA5, AA2 and AA6, AA3 and AA7, AA4 and AA8, AA5 and AA9, AA6 and AA10, or AA7 and AA11. In some embodiments, a six-amino-acid cyclic segment may be formed between AA1 and AA6, AA1B and AA7, AA2 and AA8, AA3 and AA9, AA4 and AA10, or AA5 and AA11. In some embodiments, the endpoint amino acids of the cyclic portion are Cys or D-Cys and Cys, Orn, or D-Cys. Therefore, in some embodiments, any of AA1-AA11 may be Cys, Orn, or D-Cys in cyclic peptide, depending on the location of the cyclic portion. Other cyclic connections are within the scope herein.

In some embodiments, a MC4R agonist (and/or MC3R antagonist or partial agonist) peptide descried herein is linked at the N- and/or C-terminus to one or more additional amino acids, peptides, proteins, or other carriers. In some embodiments, provided herein is a fusion of a MC4R agonist (and/or MC3R antagonist or partial agonist) peptide descried herein with one or more additional peptide or polypeptide sequences. In some embodiments, an additional peptide or polypeptide fused to the MC4R agonist (and/or MC3R antagonist or partial agonist) peptide is a carrier that confers solubility, localization (within a cell, tissue, subject, etc.), stability, cell permeability, etc. In some embodiments, an additional peptide or polypeptide fused to the MC4R agonist (and/or MC3R antagonist or partial agonist) peptide is a therapeutic peptide or polypeptide. In some embodiments, any 1-10 additional amino acids may be fused to the N-terminus or C-terminus of a MC4R agonist (and/or MC3R antagonist or partial agonist) peptide descried herein. In some embodiments, a peptide or polypeptide of 10-50, 50-100, 100-200, or more amino acids is fused to the N-terminus or C-terminus of a MC4R agonist (and/or MC3R antagonist or partial agonist) peptide descried herein.

In some embodiments, a MC4R agonist (and/or MC3R antagonist or partial agonist) is administered to a subject (e.g., by any suitable route of administration and within any suitable pharmaceutical formulation). In some embodiments, the MC4R agonist (and/or MC3R antagonist or partial agonist) peptide binds to MC4R in the subject. In some embodiments, the MC4R agonist (and/or MC3R antagonist or partial agonist) peptide binds to MC3R in the subject. In some embodiments, the activity of MC4R is enhanced (MC4R is activated) by the administration of the MC4R agonist (and/or MC3R antagonist or partial agonist) peptide. In some embodiments, the activity of MC3R is inhibited (or the activation of MC3R is reduced) by the administration of the MC4R agonist (and/or MC3R antagonist or partial agonist) peptide.

In some embodiments, methods herein comprise administering a MC4R agonist (and/or MC3R antagonist or partial agonist) peptide to a subject at risk of and/or suffering from overeating, obesity (e.g., dietary obesity, syndromic obesity (e.g., melanocortin-4 receptor haploinsufficiency), etc.), diabetes, heart disease, hypertension, sleep apnea, depression, kidney disease, arthritis, etc. In some embodiments, administration of the MC4R agonist (and/or MC3R antagonist or partial agonist) peptide results in decreased eating, decreased bodyweight, and/or other changes in observable/measurable characteristic/biomarkers for obesity, diabetes, heart disease, hypertension, sleep apnea, depression, kidney disease, arthritis, etc.

In some embodiments, the MC4R agonist (and/or MC3R antagonist or partial agonist) peptide is administered locally. In some embodiments, the MC4R agonist (and/or MC3R antagonist or partial agonist) peptide is administered systemically. In some embodiments, the MC4R agonist (and/or MC3R antagonist or partial agonist) peptide is administered in a manner such that it reaches and/or localizes in the brain. In some embodiments, the MC4R agonist (and/or MC3R antagonist or partial agonist) is administered in a manner such that it reaches and/or localizes in the hypothalamus. In some embodiments, the MC4R agonist (and/or MC3R antagonist or partial agonist) peptide is administered in a manner such that it reaches and/or localizes in AgRP neurons. In some embodiments, the MC4R agonist (and/or MC3R antagonist or partial agonist) peptide is administered in a manner such that it reaches and/or localizes in POMC neurons.

In some embodiments, a MC4R agonist (and/or MC3R antagonist or partial agonist) peptide binds MC4R selectively over other melanocortin receptors (e.g., MC1R, MC2R, MC3R, MC5R). In some embodiments, a MC4R agonist (and/or MC3R antagonist or partial agonist) peptide binds MC4R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than the binding affinity of the MC4R agonist (and/or MC3R antagonist or partial agonist) peptide with one or more other melanocortin receptors (e.g., MC1R, MC2R, MC3R, MC5R). In some embodiments, a MC4R agonist (and/or MC3R antagonist or partial agonist) peptide herein binds MC4R selectively over MC3R. In some embodiments, a MC4R agonist (and/or MC3R antagonist or partial agonist) peptide binds MC4R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than the binding affinity of the MC4R agonist (and/or MC3R antagonist or partial agonist) with MC3R.

In some embodiments, a MC4R agonist (and/or MC3R antagonist or partial agonist) peptide binds MC3R selectively over other melanocortin receptors (e.g., MC1R, MC2R, MC4R, MC5R). In some embodiments, a MC4R agonist (and/or MC3R antagonist or partial agonist) peptide binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than the binding affinity of the MC4R agonist (and/or MC3R antagonist or partial agonist) peptide with one or more other melanocortin receptors (e.g., MC1R, MC2R, MC4R, MC5R). In some embodiments, a MC4R agonist (and/or MC3R antagonist or partial agonist) peptide herein binds MC3R selectively over MC4R. In some embodiments, a MC4R agonist (and/or MC3R antagonist or partial agonist) peptide binds MC3R with an affinity that is at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than the binding affinity of the MC4R agonist (and/or MC3R antagonist or partial agonist) with MC4R.

In some embodiments, a peptide herein is a MC4R agonist and activates MC4R selectively over one or more other melanocortin receptors (e.g., MC1R, MC2R, MC3R, MC5R). In some embodiments, a peptide herein is a MC4R agonist and activates MC4R at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than one or more other melanocortin receptors (e.g., MC1R, MC2R, MC3R, MC5R). In some embodiments, a peptide herein is a MC4R agonist and enhances the activity of MC4R selectively over MC3R. In some embodiments, a peptide herein is a MC4R agonist and activates MC4R at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than MC3R.

In some embodiments, a peptide herein is a MC3R antagonist or partial agonist and inhibits the activity of MC3R selectively over one or more other melanocortin receptors (e.g., MC1R, MC2R, MC4R, MC5R). In some embodiments, a peptide herein is a MC3R antagonist or partial agonist and inhibits the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than one or more other melanocortin receptors (e.g., MC1R, MC2R, MC4R, MC5R). In some embodiments, a peptide herein is a MC3R antagonist or partial agonist and inhibits the activity of MC3R selectively over MC4R. In some embodiments, a peptide herein is a MC3R antagonist or partial agonist and inhibits the activity of MC3R at least 2-fold greater (e.g., 2×, 3×, 4×, 5×, 6×, 7×, 8×, 9×, 10×, 20×, 30×, 40×, 50×, 60×, 70×, 80×, 90×, 100×, 200×, 500×, 1000×, 2000×, 5000×, or more) than MC4R.

In some embodiments, a MC4R agonist (and/or MC3R antagonist or partial agonist) peptide is co-administered with an additional agent or therapy. In some embodiments, the co-administered agent is for the treatment or prevention of the same condition/disease/symptom as the MC3R antagonist or partial agonist peptide (e.g., overeating, obesity, diabetes, heart disease, hypertension, sleep apnea, depression, kidney disease, arthritis, etc.). In some embodiments, the co-administered agent is for the treatment or prevention of a side-effect of the MC3R antagonist or partial agonist peptide. In some embodiments, the co-administered agent is for the treatment or prevention of a comorbidity not treated of prevented by the MC3R antagonist or partial agonist peptide.

In some embodiments, the MC4R agonist (and/or MC3R antagonist or partial agonist) peptide is co-administered with psychotherapy. The term “psychotherapy” refers to use of non-pharmacological therapies a clinician or therapist uses any of a variety of techniques that involve verbal and other interactions with a patient to affect a positive therapeutic outcome. Such techniques include, but are not limited to, behavior therapy, cognitive therapy, psychodynamic therapy, psychoanalytic therapy, group therapy, family counseling, art therapy, music therapy, vocational therapy, humanistic therapy, existential therapy, transpersonal therapy, client-centered therapy (also called person-centered therapy), Gestalt therapy, biofeedback therapy, rational emotive behavioral therapy, reality therapy, response based therapy, Sandplay therapy, status dynamics therapy, hypnosis and validation therapy. Any suitable psychotherapy techniques, including those listed above, may be co-administered with a MC3R antagonist or partial agonist peptide for the treatment/prevention of appropriate conditions/diseases (e.g., overeating, obesity, diabetes, heart disease, hypertension, sleep apnea, depression, kidney disease, arthritis, etc.).

In some embodiments, the MC4R agonist (and/or MC3R antagonist or partial agonist) peptide is co-administered with one or more drugs for treating or treating/preventing obesity, preventing weight gain or overeating, or inducing weight loss, such as semaglutide (WEGOVY), Phentermine (ADIPEX, IONAMIN, SUPRENZA), diethylpropion, Phentermine-Topiramate extended release (QSYMIA), Bupropion/Naltrexone (CONTRAVE), Liraglutide (SAXENDA), etc.

In some embodiments, a MC4R agonist (and/or MC3R antagonist or partial agonist) is co-administered with an antidepressant agent. Suitable antidepressants for co-administration may include serotonin and noradrenaline reuptake inhibitors (e.g., duloxetine (Cymbalta), venlafaxine (Effexor), desvenlafaxine (Pristiq), etc.), selective serotonin reuptake inhibitors (e.g., italopram (Celexa), escitalopram (Lexapro), fluoxetine (Prozac, Sarafem), fluvoxamine (Luvox), paroxetine (Paxil), sertraline (Zoloft), etc.), tricyclic antidepressants (e.g., amitriptyline (Elavil), amoxapine-clomipramine (Anafranil), desipramine (Norpramin), doxepin (Sinequan), imipramine (Tofranil), nortriptyline (Pamelor), protriptyline (Vivactil), trimipramine (Surmontil), etc.), monoamine oxidase inhibitors (e.g., phenelzine (Nardil), tranylcypromine (Parnate), isocarboxazid (Marplan), selegiline (EMSAM, Eldepryl), etc.), noradrenaline and specific serotoninergic antidepressants (e.g., Mianserin (Tolvon), Mirtazapine (Remeron, Avanza, Zispin, etc.), etc.

In some embodiments, a MC4R agonist (and/or MC3R antagonist or partial agonist) peptide is co-administered with an antianxiety agent. Suitable antianxiety medications for co-administration may include selective serotonin reuptake inhibitors, serotonin-norepinephrine reuptake inhibitors, tricyclics, benzodiazepines (e.g., alprazolam (Xanax), chlordiazepoxide (Librium), diazepam (Valium), lorazepam (Ativan) etc.), beta-blockers (e.g., atenolol (Tenormin), propranolol (Inderal), etc.), buspirone (BuSpar), monoamine oxidase inhibitors, etc.

In some embodiments, a MC4R agonist (and/or MC3R antagonist or partial agonist) peptide is co-administered with a mood stabilizer. Suitable mood stabilizers for co-administration may include lithium, anticonvulsants (e.g., valproate, lamotrigine, carbamazepine, etc.), etc.

In some embodiments, any suitable routes and/or modes of administering the agents herein (e.g., MC4R agonist (and/or MC3R antagonist or partial agonist), co-administered agent, etc.) find use in embodiments herein. In some embodiments, the compositions and methods described herein act upon the central nervous system (CNS) and therefore routes and/or modes of administration that facilitate entry of the agents into the CNS are utilized. In some embodiments, the compositions and methods described herein act upon the brain of a subject and therefore routes and/or modes of administration that facilitate entry of the agents into the brain (e.g., allow agents to cross the blood-brain barrier) are utilized. In some embodiments, the compositions and methods described herein act upon the hypothalamus of a subject and therefore routes and/or modes of administration that facilitate delivery of the agents to the hypothalamus are utilized. In some embodiments, the compositions and methods described herein act upon the arcuate nucleus of the hypothalamus of a subject and therefore routes and/or modes of administration that facilitate delivery of the agents to the arcuate nucleus are utilized. In some embodiments, the compositions and methods described herein act upon the AgRP neurons of a subject and therefore routes and/or modes of administration that facilitate delivery of the agents to AgRP neurons are utilized. In some embodiments, the compositions and methods described herein act upon the POMC neurons of a subject and therefore routes and/or modes of administration that facilitate delivery of the agents to POMC neurons are utilized.

In some embodiments, routes of administration, formation of the desired agent, and the pharmaceutical composition are selected to provide efficient and effective delivery. In some embodiments, the therapeutic agents herein (e.g., MC4R agonist (and/or MC3R antagonist or partial agonist) peptide, co-administered agent, etc.) are provided in pharmaceutical formulations for administration to a subject by a suitable route. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein (e.g., comprising a MC3R antagonist or partial agonist peptide, a co-administered agent, etc.) are formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

One may administer the compounds and/or compositions in a local rather than systemic manner, for example, via injection of the compound directly into an organ or tissue, often in a depot preparation or sustained release formulation. Such long-acting formulations may be administered by implantation (for example subcutaneously or intramuscularly) or by intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended-release formulation, or in the form of an intermediate release formulation.

Pharmaceutical preparations for oral use can be obtained by mixing one or more solid excipients with the therapeutic agent (e.g., MC4R agonist (and/or MC3R antagonist or partial agonist) peptide, a co-administered agent, etc.) with any suitable substituents and functional groups disclosed herein, optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as; polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In some embodiments, agents are delivered by inhalation. For administration by inhalation, the agents described herein (e.g., MC4R agonist (and/or MC3R antagonist or partial agonist) peptide, a co-administered agent, etc.) may be in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Buccal formulations that include the agents described herein (e.g., MC4R agonist (and/or MC3R antagonist or partial agonist) peptide, a co-administered agent, etc.) may be administered using a variety of formulations which include, but are not limited to, U.S. Pat. Nos. 4,229,447, 4,596,795, 4,755,386, and 5,739,136.

In some embodiments, the agents described herein (e.g., antagonist or partial agonist agonist peptide, a co-administered agent, etc.) are delivered transdermally. Transdermal formulations described herein may be administered using a variety of devices including but not limited to, U.S. Pat. Nos, 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336, 168, 5,665,378, 5,837,280, 5,869,090, 6,923,983, 6,929,801 and 6,946,144; incorporated by reference in their entireties.

In some embodiments, the agents described herein (e.g., MC4R agonist (and/or MC3R antagonist or partial agonist) peptide, a co-administered agent, etc.) are delivered by parenteral administration (e.g., intramuscular, subcutaneous, intravenous, epidural, intracerebral, intracerebroventricular, etc.). Formulations suitable for parenteral administration may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Agents described herein (e.g., MC4R agonist (and/or MC3R antagonist or partial agonist) peptide, a co-administered agent, etc.) may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

In certain embodiments, delivery systems for pharmaceutical agents (e.g., MC4R agonist (and/or MC3R antagonist or partial agonist) agonist, a co-administered agent, etc.) may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, an agent (e.g., MC4R agonist (and/or MC3R antagonist or partial agonist) peptide, a co-administered agent, etc.) is administered in a therapeutically effective amount. Thus, a therapeutically effective amount is an amount that is capable of at least partially preventing or reversing a disease, disorder, or symptoms thereof. The dose required to obtain an effective amount may vary depending on the agent, formulation, disease or disorder, and individual to whom the agent is administered.

Determination of effective amounts may involve in vitro assays in which varying doses of agent are administered to cells in culture and the concentration of agent effective for ameliorating some or all symptoms is determined in order to calculate the concentration required in vivo. Effective amounts may also be based in in vivo animal studies.

Pharmaceutical compositions may be in unit dosage forms suitable for single administration of precise dosages. In unit dosage form, the formulation is divided into unit doses containing appropriate quantities of one or more agents (e.g., MC4R agonist (and/or MC3R antagonist or partial agonist) peptide, a co-administered agent, etc.).

Dosing and administration regimes are tailored by the clinician, or others skilled in the pharmacological arts, based upon well-known pharmacological and therapeutic considerations including, but not limited to, the desired level of therapeutic effect, and the practical level of therapeutic effect obtainable.

In some embodiments, and upon the clinician's discretion, the administration of the compounds may be administered for an extended period of time, including throughout the duration of the patient's life in order to treat the disorder or ameliorate or otherwise control or limit the symptoms of the patient's disease.

In a case wherein the patient's status does improve, upon the clinician's discretion the administration of the agents (e.g., MC4R agonist (and/or MC3R antagonist or partial agonist) peptide, a co-administered agent, etc.) may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, once improvement of the patient's symptoms/disorder/condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

In some embodiments, the amount of a given agent that will correspond to such an amount will vary depending upon factors such as the particular compound, disease and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02—about 5000 mg per day, in some embodiments, about 1—about 1500 mg per day. The desired dose may conveniently be presented in a single dose or as divided doses administered simultaneously (or over a short period of time) or at appropriate intervals, for example as two, three, four or more sub-doses per day.

As discussed above, provided in certain embodiments herein are combination therapies in which a MC4R agonist (and/or MC3R antagonist or partial agonist) peptide is co-administered with an additional agent for the treatment of the disorder/condition, a side effect of the primary agent, or a comorbidity of the disorder/condition. Co-administered agents do not have to be administered in the same pharmaceutical composition, and may, because of different physical and chemical characteristics, have to be administered by different routes. Co-administered agents may be administered concurrently (in the same or separate formulations/compositions) or at separate times (separated by minutes, hours, days, etc.) The co-administered agents may be administered concurrently (e.g., simultaneously, essentially simultaneously or within the same treatment protocol) or sequentially, depending upon the nature of the disease, disorder, or condition, the condition of the patient, and the actual choice of agent used. The determination of the order of administration, and the number of repetitions of administration of each therapeutic agent during a treatment protocol, is well within the knowledge of the clinician after evaluation of the disease being treated and the condition of the patient.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered agents will of course vary depending on the type of co-drug employed, on the specific drug employed, on the disease being treated and so forth. In addition, when co-administered with one or more biologically active agents, the compound provided herein may be administered either simultaneously with the biologically active agent(s), or sequentially.

### EXPERIMENTAL

### Example 1

### Pharmacological In Vitro ASSAYS

Determination of intracellular cAMP levels in live cells: A genetically-encoded cAMP split-luciferase reporter stably-expressing cell line (Promega, Madison, WI) (Binkowski et al., 2011, ACS chemical biology 6, 1193-1197; incorporated by reference in its entirety) was used as the basis for the generation of stable clones expressing the human MC4R receptor (a gift from Promega), or the human MC3R (generated in-house by clonal selection). The stable cell lines were grown and maintained in selection media consisting of Dulbecco's modified Eagle media (DMEM) with 4.5 g/l D-glucose, and 4 mM L-glutamine (Thermo Fisher Scientific, Waltham, MA), supplemented with 10% fetal bovine serum, 100 units/ml penicillin, 100 μg/ml streptomycin, 2.5 μg/ml amphotericin B, 200 μg/ml hygromycin B (for positive selection of the GScAMP22f luciferase reporter), and Geneticin™ (G418) 700 μg/ml (for MC3R or MC4R selection). Actual serum concentration during the assay is estimated to be about 1%. Cell line identity is routinely verified by qPCR and MC3R-and MC4R-specific oligonucleotides.

The assay for the determination of the cAMP response in live cells was described previously. (Yu et al., 2020, Science 368, 428-433; incorporated by reference in its entirety). Cells were seeded at a density of 20,000 cells per well using 384-well poly-D lysine-coated, clear bottom, and black-wall assay plates (Corning Inc. Corning, NJ). Cells were allowed to attach to the plates for 18 to 24 h after which growth media was removed and 20 μl of 4% D-luciferin (Promega) in CO2 independent, serum-free medium (Thermo Fisher Scientific) was added to each well. The luciferase substrate was allowed to permeate the cells for 120 min at 37° C. Intracellular cAMP levels were measured using an FDSS 7000EX Functional Drug Screening System (Hamamatsu Photonics, Hamamatsu, Japan) in the Center for Chemical Genomics at the Life Sciences Institute. This instrument allowed the in-line addition of test-peptides and receptor agonists while simultaneously acquiring the luminescence signal from live cells. Assay read steps were set as follows: baseline acquisition of 2 min, the addition of 10 μl of varying 3× concentrations of test-peptides or vehicle followed by 11 min measurement (measurement window 1), and 10 μl addition of 4× concentration of the endogenous melanocortin agonist α-MSH (Bachem, Bubendorf, Switzerland) followed by an additional 11 min response measurement (measurement window 2). The resulting final concentration of α-MSH was close to the respective receptor EC90 dose for each receptor. Intraplate concentration response curves for α-MSH and SHU-9119 (Phoenix Pharmaceuticals, Burlingame CA) were included as reference controls. A submaximal forskolin (20 μM) concentration was also included to serve as a normalization reference to account for cell number variations and differences in assay transducer efficiency between cell lines.

With this set up it was possible to evaluate the direct effect of the test-peptides on the MC3R and MC4R cell lines during measurement window 1, while the antagonist profile in the presence of EC90 α-MSH was determined on measurement window 2. For data analysis baseline luminescence (i.e. the maximum luminescence signal from the initial 0 to 2 min window) was subtracted from the maximum luminescence obtained during measurement window 1 (2 to 13 min) and measurement window 2 (13 to 24 min) to yield the test-peptide elicited responses. EC50 or IC50 potency values were determined by non-linear regression by fitting the data to a sigmoid four-parameter variable slope model using the GraphPad Prism version 8.4 software package (San Diego CA).

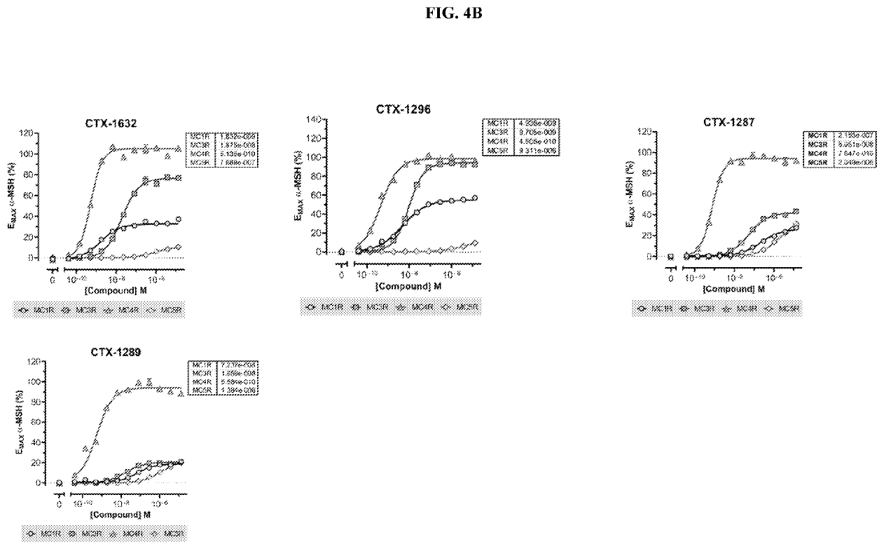

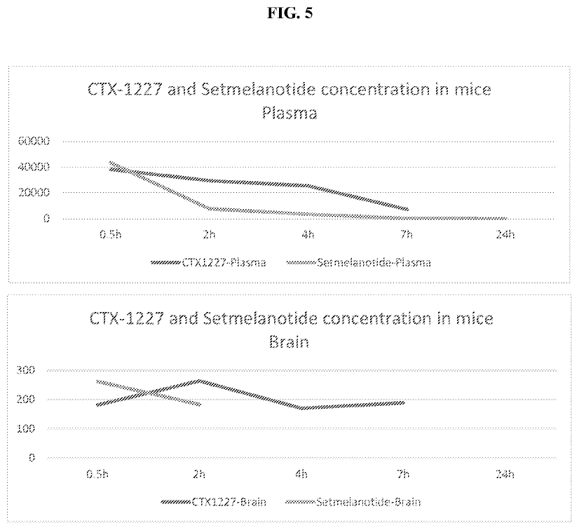

Exemplary results of pharmacological in vitro assays are provided in FIGS. 2 and 4, and Tables 1-4.

TABLE 1
MC3R and MC4R cAMP (EC50) and % maximal
activation for exemplary MC4R agonists.

| | | | | | MC3R | MC4R |
|---|---|---|---|---|---|---|
| | | | | | cAMP | cAMP |
| | N | | SEQ | C- | EC50 | EC50 |
| Peptide | term. | | ID | term. | (% max | (% max |
| code | Mod. | Sequence | NO. | Mod. | activation) | activation) |
| CTX- | Chloro | Thioether-cyclo((D- | 3 | NH2 | 130 nM | 0.65 nM |
| 1202 | acetyl | Ala-His-D-Phe-Arg- | | | (101%) | (104%) |
| | | Trp-Cys) | | | | |
| CTX- | Chloro | Thioether-cyclo(D- | 4 | NH2 | 2,612 nM | 16 nM |
| 1203 | acetyl | Ala-Nme-His-D- | | | (107%) | (109%) |
| | | Phe-Arg-Nme-Trp- | | | | |
| | | Cys) | | | | |
| CTX- | Ac | cyclo(Cys-Glu-His- | 5 | NH2 | 9.0 nM | 1.0 nM |
| 1205 | | D-Phe-Arg-Trp- | | | (92%) | (99%) |
| | | Cys) | | | | |
| CTX- | Ac | cyclo(Cys-Gly-His- | 6 | NH2 | 12 nM | 0.52 |
| 1206 | | D-Phe-Arg-Trp- | | | (94%) | (101%) |
| | | Cys) | | | | |
| CTX- | Ac | cyclo(Cys-D-Ala- | 7 | NH2 | 8.1 nM | 0.91 nM |
| 1207 | | His-D-Phe-Arg-Trp- | | | (89%) | (96%) |
| | | Cys) | | | | |
| CTX- | Ac | Arg-cyclo(Cys-D- | 8 | NH2 | 2.1 nM | 0.38 nM |
| 1211 | | Ala-Arg-D-Phe-Arg- | | | (91%) | (96%) |
| | | Trp-Cys) | | | | |
| CTX- | Ac | Arg-cyclo(Cys-D- | 9 | NH2 | 9.4 nM | 47 nM |
| 1213 | | Ala-His-Phe-Arg- | | | (97%) | (104%) |
| | | Trp-Cys) | | | | |
| CTX- | Ac | Arg-Nle-cyclo(Cys- | 10 | NH2 | 2.8 nM | 0.3 nM |
| 1227 | | His-D-Phe-Arg-Trp- | | | (96%) | (103%) |
| | | Cys) | | | | |
| CTX- | Ac | Arg-Nle-cyclo(Cys- | 11 | NH2 | 1.6 nM | 0.2 nM |
| 1228 | | Arg-D-Phe-Arg-Trp- | | | (93%) | (102%) |
| | | Cys) | | | | |
| CTX- | Ac | Arg-cyclo(Cys-D- | 12 | NH2 | 44 nM | 692 |
| 1257 | | Ala-Arg-D-Phe-Arg- | | | (88%) | (94%) |
| | | D-Phe-Cys) | | | | |
| CTX- | Ac | Arg-cyclo(Cys-D- | 13 | NH2 | 2.5 nM | 1.8 nM |
| 1262 | | Ala-His-D-Phe-Arg- | | | (65%) | (88%) |
| | | D-Trp-Cys) | | | | |
| CTX- | Ac | Arg-cyclo(Cys-D- | 14 | NH2 | 11 nM | 6.5 nM |
| 1263 | | Ala-Arg-D-Phe-Arg- | | | (69%) | (93%) |
| | | D-Trp-Cys) | | | | |
| CTX- | Ac | Arg-cyclo(Cys-D- | 15 | NH2 | 3.7 nM | 13 nM |
| 1264 | | Ala-Arg-D-Phe-Arg- | | | (44%) | (80%) |
| | | D-Nal(2′)-Cys) | | | | |
| CTX- | Ac | Arg-cyclo(Cys-D- | 16 | NH2 | 29.9 nM | 703.1 nM |
| 1265 | | Ala-Arg-D-Phe-Arg- | | | (60%) | (71%) |
| | | D-Phe-Cys) | | | | |
| CTX- | Ac | Arg-cyclo(Cys-Ala- | 17 | NH2 | 12 nM | 3 nM |
| 1267 | | Arg-D-Phe-Arg-Trp- | | | (40%) | (96%) |
| | | Cys) | | | | |
| CTX- | Ac | Arg-cyclo(Cys-Ala- | 18 | NH2 | 211 nM | 7 nM |
| 1268 | | Arg-D-Phe-Arg-D- | | | (44%) | (96%) |
| | | Trp-Cys) | | | | |
| CTX- | Ac | Arg-cyclo(Cys- | 19 | NH2 | 121 nM | 13 nM |
| 1269 | | NMe-Ala-Arg-D- | | | (39%) | (92%) |
| | | Phe-Arg-D-Trp- | | | | |
| | | Cys) | | | | |
| CTX- | Ac | Arg-cyclo(Cys-Aib- | 20 | NH2 | 52 nM | 27 nM |
| 1270 | | Arg-D-Phe-Cit-Trp- | | | (14%) | (98%) |
| | | Cys) | | | | |
| CTX- | Ac | Arg-cyclo(Cys-D- | 21 | NH2 | 418 nM | 36 nM |
| 1278 | | Ala-Arg-aMe-D- | | | (86%) | (105%) |
| | | Phe-Arg-Trp-Cys) | | | | |
| CTX- | Ac | Arg-cyclo(Cys-D- | 22 | NH2 | 28 nM | 97 nM |
| 1281 | | Ala-Arg-D-Phe-Cit- | | | (44%) | (92%) |
| | | D-Trp-Cys) | | | | |
| CTX- | Ac | Arg-cyclo(Cys-D- | 23 | NH2 | 1,035 nM | 128 nM |
| 1284 | | Ala-Arg-D-Phe-N- | | | (45%) | (91%) |
| | | Me-Arg-D-Trp-Cys) | | | | |
| CTX- | Ac | Arg-cyclo(Cys-D- | 24 | NH2 | 4.9 nM | 1.7 nM |
| 1285 | | Ala-Cit-D-Phe-Arg- | | | (28%) | (94%) |
| | | Trp-Cys) | | | | |
| CTX- | Ac | Arg-cyclo(Cys-D- | 25 | NH2 | 568 nM | 262 nM |
| 1286 | | Ala-Cit-D-Phe-Arg- | | | (24%) | (91%) |
| | | D-Trp-Cys) | | | | |
| CTX- | Ac | Arg-cyclo(Cys-Ala- | 26 | NH2 | 50 nM | 0.77 nM |
| 1287 | | Cit-D-Phe-Arg-Trp- | | | (43%) | (94%) |
| | | Cys) | | | | |
| CTX- | Ac | Arg-cyclo(Cys-Ala- | 27 | NH2 | 1,250 nM | 77 nM |
| 1288 | | Cit-D-Phe-Arg-D- | | | (36%) | (87%) |
| | | Trp-Cys) | | | | |
| CTX- | Ac | Arg-cyclo(Cys-Aib- | 28 | NH2 | 17 nM | 0.6 nM |
| 1289 | | Cit-D-Phe-Arg-Trp- | | | (21%) | (93%) |
| | | Cys) | | | | |
| CTX- | Ac | Arg-cyclo(Cys-Aib- | 29 | NH2 | 1,035 nM | 112 nM |
| 1290 | | Cit-D-Phe-Arg-D- | | | (20%) | (88%) |
| | | Trp-Cys) | | | | |
| CTX- | Ac | Arg-cyclo(Cys-Aib- | 30 | NH2 | 1.5 nM | 0.18 nM |
| 1291 | | His-D-Phe-Arg-Trp- | | | (91%) | (98%) |
| | | Cys) | | | | |
| CTX- | Ac | Arg-cyclo(Cys-Aib- | 31 | NH2 | 3.3 nM | 0.16 nM |
| 1292 | | Arg-D-Phe-Arg-Trp- | | | (76%) | 90%) |
| | | Cys) | | | | |
| CTX- | Ac | Arg-cyclo(Cys-Gly- | 32 | NH2 | 2.5 nM | 0.25 nM |
| 1293 | | Arg-D-Phe-Arg-Trp- | | | (95%) | (87%) |
| | | Cys) | | | | |
| CTX- | Ac | Arg-cyclo(Cys-Gly- | 33 | NH2 | 40 nM | 3.5 nM |
| 1294 | | Cit-D-Phe-Arg-Trp- | | | (33%) | (102%) |
| | | Cys) | | | | |
| CTX- | Ac | Arg-cyclo(Cys-D- | 34 | NH2 | 1.6 nM | 0.25 nM |
| 1295 | | Abu-His-D-Phe- | | | (97%) | (102%) |
| | | Arg-Trp-Cys) | | | | |
| CTX- | Ac | Arg-cyclo(Cys-Aib- | 35 | NH2 | 10 nM | 0.45 nM |
| 1296 | | His-D-Phe-Arg-D- | | | (93%) | (99%) |
| | | Trp-Cys) | | | | |
| CTX- | Ac | Arg-cyclo(Cys-Aib- | 36 | NH2 | >10,000 | 3.3 nM |
| 1297 | | Arg-D-Tyr(4-OMe)- | | | nM | (97%) |
| | | Arg-D-Trp-Cys) | | | | |
| CTX- | Ac | Arg-cyclo(Cys-Aib- | 37 | NH2 | >10,000 | 3.3 nM |
| 1298 | | Arg-D-Tyr(4-OMe)- | | | nM | (93%) |
| | | Arg-Phe-Cys) | | | | |
| CTX- | Ac | Arg-cyclo(Cys-Aib- | 38 | NH2 | >10,000 | 65 nM |
| 1299 | | Cit-D-Tyr(4-OMe)- | | | nM | (78%) |
| | | Arg-Phe-Cys) | | | | |
| CTX- | Ac | Arg-cyclo(Cys-Aib- | 39 | NH2 | 26 nM | 11 nM |
| 1601 | | Arg-D-Phe-Arg- | | | (73%) | (97%) |
| | | Phe-Cys) | | | | |
| CTX- | Ac | Arg-cyclo(Cys-Aib- | 40 | NH2 | 251 nM | 73 nM |
| 1602 | | Orn-D-Phe-Arg- | | | (80%) | (97%) |
| | | Phe-Cys) | | | | |
| CTX- | Ac | Arg-cyclo(Cys-Aib- | 41 | NH2 | >10,000 | 562 nM |
| 1603 | | homo-Cit-D-Phe- | | | nM | (91%) |
| | | Arg-Phe-Cys) | | | | |
| CTX- | Ac | Arg-cyclo(Cys-Aib- | 42 | NH2 | 167 nM | 1,524 nM |
| 1604 | | Pal(2′)-D-Phe-Arg- | | | (12%) | 96%) |
| | | Phe-Cys) | | | | |
| CTX- | Ac | Arg-cyclo(Cys-Aib- | 43 | NH2 | >10,000 | 33 nM |
| 1605 | | Pal(3′)-D-Phe-Arg- | | | nM | (85%) |
| | | Phe-Cys) | | | | |
| CTX- | Ac | Arg-cyclo(Cys-Aib- | 44 | NH2 | 82 nM | 133 nM |
| 1606 | | Pal(4′)-D-Phe-Arg- | | | (7.69%) | (92%) |
| | | Phe-Cys) | | | | |
| CTX- | Ac | Arg-Nle-cyclo(Cys- | 45 | NH2 | 14 nM | 1.8 nM |
| 1607 | | Cit-D-Phe-Arg-Trp- | | | (47%) | (93%) |
| | | Cys) | | | | |
| CTX- | Ac | Arg-Nle-cyclo(Cys- | 46 | NH2 | 274 nM | 288 nM |
| 1608 | | Cit-D-Phe-Arg-D- | | | (19%) | (79%) |
| | | Trp-Cys) | | | | |
| CTX- | Ac | Arg-Nle-cyclo(Cys- | 47 | NH2 | 407 nM | 86 nM |
| 1609 | | Cit-Phe-Arg-Trp- | | | (65%) | (96%) |
| | | Cys) | | | | |
| CTX- | Ac | Arg-Nle-cyclo(Cys- | 48 | NH2 | 163 nM | 247 nM |
| 1610 | | His-Nal(1′)-Arg-Trp- | | | (67%) | (90%) |
| | | Cys) | | | | |
| CTX- | Ac | Arg-Nle-cyclo(Cys- | 49 | NH2 | 11 nM | 1.6 nM |
| 1611 | | Arg-D-Nal(1′)-Arg- | | | (58%) | (67%) |
| | | Trp-Cys) | | | | |
| CTX- | Ac | Arg-Nle-cyclo(Cys- | 50 | NH2 | 61 nM | 16 nM |
| 1616 | | Arg-D-Phe-Arg-D- | | | (78%) | (93%) |
| | | Trp-Cys) | | | | |
| CTX- | Ac | Arg-Nle-cyclo(Cys- | 51 | NH2 | 2.3 nM | 0.21 nM |
| 1617 | | Orn-D-Phe-Arg- | | | (102%) | (102%) |
| | | Trp-Cys) | | | | |
| CTX- | Ac | Arg-Nle-cyclo(Cys- | 52 | NH2 | 29 nM | 1.8 nM |
| 1618 | | homo-Cit-D-Phe- | | | (33%) | (94%) |
| | | Arg-Trp-Cys) | | | | |
| CTX- | Ac | Arg-Nle-cyclo(Cys- | 53 | NH2 | 33 nM | 2.6 nM |
| 1619 | | Pal(2′)-D-Phe-Arg- | | | (84%) | (99%) |
| | | Trp-Cys) | | | | |
| CTX- | Ac | Arg-Nle-cyclo(Cys- | 54 | NH2 | 58 nM | 4.7 nM |
| 1620 | | Pal(3′)-D-Phe-Arg- | | | (39%) | (91%) |
| | | Trp-Cys) | | | | |
| CTX- | Ac | Arg-Nle-cyclo(Cys- | 55 | NH2 | 36 nM | 2.9 nM |
| 1621 | | Pal(4′)-D-Phe-Arg- | | | (15%) | (92%) |
| | | Trp-Cys) | | | | |
| CTX- | Ac | Arg-Nle-cyclo(Cys- | 56 | NH2 | 13 nM | 41 nM |
| 1622 | | Arg-D-Phe-Arg- | | | (100%) | (101%) |
| | | Phe-Cys) | | | | |
| CTX- | Ac | Arg-Nle-cyclo(Cys- | 57 | NH2 | 3.9 nM | 1.7 nM |
| 1623 | | Arg-D-Phe(4-Br)- | | | (62%) | (73%) |
| | | Arg-Phe-Cys) | | | | |
| CTX- | Ac | Arg-Nle-cyclo(Cys- | 58 | NH2 | 5.6 nM | 12 nM |
| 1624 | | Arg-D-Tyr(4-OMe)- | | | (66%) | (83%) |
| | | Arg-Phe-Cys) | | | | |
| CTX- | Ac | Arg-Nle-cyclo(Cys- | 59 | NH2 | 72 nM | 38 nM |
| 1625 | | His-D-Phe-Arg- | | | (92%) | (101%) |
| | | Phe-Cys) | | | | |
| CTX- | Ac | Arg-Nle-cyclo(Cys- | 60 | NH2 | 678 nM | 425 nM |
| 1626 | | Cit-D-Phe-Arg-Phe- | | | (51%) | (83%) |
| | | Cys) | | | | |
| CTX- | Ac | cyclo(Cys-D-Ala- | 61 | NH2 | 3.7 nM | 1.4 nM |
| 1627 | | Arg-D-Phe-Arg-Trp- | | | (98%) | (104%) |
| | | Cys) | | | | |
| CTX- | Ac | cyclo(Cys-D-Ala- | 62 | NH2 | 92 nM | 14 nM |
| 1628 | | Cit-D-Phe-Arg-Trp- | | | (45%) | (99%) |
| | | Cys) | | | | |
| CTX- | Ac | cyclo(Cys-Gly-Arg- | 63 | NH2 | 13 nM | 0.67 nM |
| 1629 | | D-Phe-Arg-Trp- | | | (83%) | (102%) |
| | | Cys) | | | | |
| CTX- | Ac | cyclo(Cys-Gly-Cit- | 64 | NH2 | 614 nM | 22 nM |
| 1630 | | D-Phe-Arg-Trp- | | | (35%) | (102%) |
| | | Cys) | | | | |
| CTX- | Ac | cyclo(Cys-Aib-His- | 65 | NH2 | 2.9 nM | 0.43 nM |
| 1631 | | D-Phe-Arg-Trp- | | | (100%) | (107%) |
| | | Cys) | | | | |
| CTX- | Ac | cyclo(Cys-Aib-Arg- | 66 | NH2 | 19 nM | 0.51 nM |
| 1632 | | D-Phe-Arg-Trp- | | | (77%) | (105%) |
| | | Cys) | | | | |
| CTX- | Ac | cyclo(Cys-Aib-Cit- | 67 | NH2 | 290 nM | 2.8 nM |
| 1633 | | D-Phe-Arg-Trp- | | | (45%) | (110%) |
| | | Cys) | | | | |
| CTX- | Ac | cyclo(Cys-Aib-His- | 68 | NH2 | 56 nM | 8.5 nM |
| 1634 | | D-Phe-Arg-Phe- | | | (94%) | (107%) |
| | | Cys) | | | | |
| CTX- | Ac | cyclo(Cys-Aib-Arg- | 69 | NH2 | 380 nM | 67 nM |
| 1635 | | D-Phe-Arg-Phe- | | | (70%) | (112%) |
| | | Cys) | | | | |
| CTX- | Ac | cyclo(Cys-Aib-Cit- | 70 | NH2 | 7,464 nM | 791 nM |
| 1636 | | D-Phe-Arg-Phe- | | | (44%) | (102%) |
| | | Cys) | | | | |
| CTX- | Ac | cyclo(Cys-Gly-His- | 71 | NH2 | 68 nM | 47 nM |
| 1637 | | D-Phe-Arg-Phe- | | | (98%) | (108%) |
| | | Cys) | | | | |
| CTX- | Ac | cyclo(Cys-Gly-Arg- | 72 | NH2 | 408 nM | 351 nM |
| 1638 | | D-Phe-Arg-Phe- | | | (75%) | (110%) |
| | | Cys) | | | | |
| CTX- | Ac | cyclo(Cys-Gly-Cit- | 73 | NH2 | 7,691 nM | 2,729 nM |
| 1639 | | D-Phe-Arg-Phe- | | | (44%) | (88%) |
| | | Cys) | | | | |
| CTX- | Chloro | Thioether-cyclo(D- | 74 | NH2 | 83 nM | 2.9 nM |
| 1642 | acetyl | Ala-Arg-D-Phe-Arg- | | | (90%) | (107%) |
| | | Trp-Cys) | | | | |
| CTX- | Chloro | Thioether-cyclo(D- | 75 | NH2 | 2,547 nM | 19 nM |
| 1643 | acetyl | Ala-Cit-D-Phe-Arg- | | | (81%) | (111%) |
| | | Trp-Cys) | | | | |
| CTX- | Chloro | Thioether- | 76 | NH2 | 120 nM | 2.3 nM |
| 1644 | acetyl | cyclo(Gly-His-D- | | | (107%) | (111%) |
| | | Phe-Arg-Trp-Cys) | | | | |
| CTX- | Chloro | Thioether- | 77 | NH2 | 68 nM | 1.6 nM |
| 1645 | acetyl | cyclo(Gly-Arg-D- | | | (97%) | (110%) |
| | | Phe-Arg-Trp-Cys) | | | | |
| CTX- | Chloro | Thioether- | 78 | NH2 | 2,421 nM | 13 nM |
| 1646 | acetyl | cyclo(Gly-Cit-D- | | | (70%) | (103%) |
| | | Phe-Arg-Trp-Cys) | | | | |
| CTX- | Chloro | Thioether- | 79 | NH2 | 21 nM | 0.68 nM |
| 1647 | acetyl | cyclo(Aib-His-D- | | | (102%) | (109%) |
| | | Phe-Arg-Trp-Cys) | | | | |
| CTX- | Chloro | Thioether- | 80 | NH2 | 72 nM | 1.1 nM |
| 1648 | acetyl | cyclo(Aib-Arg-D- | | | (91%) | (101%) |
| | | Phe-Arg-Trp-Cys) | | | | |
| CTX- | Chloro | Thioether- | 81 | NH2 | 479 nM | 6.6 nM |
| 1649 | acetyl | cyclo(Aib-Cit-D- | | | (79%) | (97%) |
| | | Phe-Arg-Trp-Cys) | | | | |
| CTX- | Chloro | Thioether- | 82 | NH2 | 15,171 nM | 288 nM |
| 1650 | acetyl | cyclo(Aib-His-D- | | | (125%) | (113%) |
| | | Phe-Arg-Phe-Cys) | | | | |
| CTX- | Chloro | Thioether- | 83 | NH2 | >10,000 | 1,194 nM |
| 1651 | acetyl | cyclo(Aib-Arg-D- | | | nM | (92%) |
| | | Phe-Arg-Phe-Cys) | | | | |
| CTX- | Chloro | Thioether- | 84 | NH2 | >10,000 | >10,000 |
| 1652 | acetyl | cyclo(Aib-Cit-D- | | | nM | nM |
| | | Phe-Arg-Phe-Cys) | | | | |
| CTX- | Chloro | Thioether- | 85 | NH2 | >10,000 | 1,225 nM |
| 1653 | acetyl | cyclo(Gly-His-D- | | | nM | (83%) |
| | | Phe-Arg-Phe-Cys) | | | | |
| CTX- | Chloro | Thioether- | 86 | NH2 | 5,888 nM | 1,089 nM |
| 1654 | acetyl | cyclo(Gly-Arg-D- | | | (43%) | (81%) |
| | | Phe-Arg-Phe-Cys) | | | | |
| CTX- | Chloro | Thioether- | 87 | NH2 | >10,000 | >10,000 |
| 1655 | acetyl | cyclo(Gly-Cit-D- | | | nM | nM |
| | | Phe-Arg-Phe-Cys) | | | | |
| CTX- | Chloro | Thioether-cyclo(D- | 88 | NH2 | 2,301 nM | 1,400 nM |
| 1656 | acetyl | Ala-His-D-Phe-Arg- | | | (86%) | (98%) |
| | | Phe-Cys) | | | | |
| CTX- | Chloro | Thioether-cyclo(D- | 89 | NH2 | 8,810 nM | 2,472 nM |
| 1657 | acetyl | Ala-Arg-D-Phe-Arg- | | | 30%) | (78%) |
| | | Phe-Cys) | | | | |
| CTX- | Chloro | Thioether-cyclo(D- | 90 | NH2 | >10,000 | >10,000 |
| 1658 | acetyl | Ala-Cit-D-Phe-Arg- | | | nM | nM |
| | | Phe-Cys) | | | | |
| CTX- | Ac | Nle-cyclo(Cys-His- | 91 | NH2 | 39 nM | 7,211 nM |
| 2128 | | Nal(2′)-Arg-D-Trp- | | | (99%) | (87%) |
| | | Cys)-Arg-Phe-Gly | | | | |

TABLE 2
MC3R and MC4R cAMP EC50 and % maximal activation for exemplary MC4R
agonists with significant MC3R antagonism or partial agonism

| | | | | | MC3R cAMP | MC4R cAMP |
|---|---|---|---|---|---|---|
| Peptide | N-term. | | SEQ | C-term. | EC50 (% max | EC50 (% max |
| code | Mod. | Sequence | ID NO. | Mod. | activation) | activation) |
| CTX-1212 | Ac | Arg-cyclo(Cys-D- | 92 | NH2 | 28 nM | 393 nM |
| | | Ala-Arg-Phe(4-Br)- | | | (0.1%) | (55%) |
| | | Arg-Trp-Cys) | | | | |
| CTX-1214 | Ac | Arg-cyclo(Cys-D- | 93 | NH2 | 4.1 nM | 2.6 nM |
| | | Ala-Arg-D-Phe(4- | | | (15%) | (74%) |
| | | Br)-Arg-D-Trp-Cys) | | | | |
| CTX-1215 | Ac | Arg-cyclo(Cys-D- | 94 | NH2 | 27 nM | 592 nM |
| | | Ala-Arg-Phe(4-Br)- | | | (2.1%) | (56%) |
| | | Arg-D-Trp-Cys) | | | | |
| CTX-1216 | Ac | Arg-cyclo(Cys-D- | 95 | NH2 | 3.5 nM | 1.8 nM |
| | | Ala-Arg-D-Phe(4- | | | (11%) | (69%) |
| | | Br)-Arg-D-Trp-Cys)- | | | | |
| | | Lys-Pro-Val | | | | |
| CTX-1217 | Ac | Arg-cyclo(Cys-D- | 96 | NH2 | 4.0 nM | 1.5 nM |
| | | Ala-Arg-D-Phe(4- | | | (13%) | (69%) |
| | | Br)-Arg-D-Trp-Cys)- | | | | |
| | | Lys-Pro | | | | |
| CTX-1218 | Ac | Arg-cyclo(Cys-D- | 97 | NH2 | 4.0 | 2.2 |
| | | Ala-Arg-D-Phe(4-Br)- | | | (16%) | (77%) |
| | | Arg-D-Trp-Cys)-Lys | | | | |
| CTX-1219 | Ac | Arg-cyclo(Cys-D- | 98 | free | 4.5 nM | 2.3 nM |
| | | Ala-Arg-D-Phe(4- | | | (20%) | (76%) |
| | | Br)-Arg-D-Trp-Cys)- | | | | |
| | | Arg-Phe-Gly | | | | |
| CTX-1220 | Ac | Arg-cyclo(Cys-D- | 99 | NH2 | 7.2 nM | 1.87 nM |
| | | Ala-Arg-D-Phe(4- | | | (21%) | (76%) |
| | | Br)-Arg-D-Trp-Cys)- | | | | |
| | | Arg-Phe | | | | |
| CTX-1222 | Ac | Arg-cyclo(Cys-D- | 100 | NH2 | 9.8 nM | 30 nM |
| | | Ala-Arg-D-Phe(4- | | | (38%) | (79%) |
| | | Br)-Arg-D-Trp-Cys)- | | | | |
| | | Arg-D-Phe | | | | |
| CTX-1226 | R2 | Arg-cyclo(Cys-D- | 101 | NH2 | 12 nM | 3.8 nM |
| | | Ala-Arg-D-Phe(4- | | | (29%) | (71%) |
| | | Br)-Arg-D-Trp-Cys) | | | | |
| CTX-1229 | Ac | Arg-Nle-cyclo(Cys- | 102 | NH2 | 3.5 nM | 18 nM |
| | | His-D-Phe(4-Br)- | | | (16%) | (31%) |
| | | Arg-Trp-Cys) | | | | |
| CTX-1230 | Ac | Arg-Nle-cyclo(Cys- | 103 | NH2 | 3.9 nM | 1.4 nM |
| | | Arg-D-Phe(4-Br)- | | | (23%) | (27%) |
| | | Arg-Trp-Cys) | | | | |
| CTX-1231 | Ac | Arg-Nle-cyclo(Cys- | 104 | NH2 | 7.0 nM | 2.3 nM |
| | | His-D-Phe(4-Br)- | | | (59%) | (64%) |
| | | Arg-D-Trp-Cys) | | | | |
| CTX-1232 | Ac | Arg-Nle-cyclo(Cys- | 105 | NH2 | 6.0 nM | 2.4 nM |
| | | Arg-D-Phe(4-Br)- | | | (41%) | (46%) |
| | | Arg-D-Trp-Cys) | | | | |
| CTX-1233 | Ac | Arg-Nle-cyclo(Cys- | 106 | NH2 | 4.1 nM | 2.2 nM |
| | | His-D-Phe(4-I)-Arg- | | | (22%) | (16%) |
| | | D-Trp-Cys) | | | | |
| CTX-1234 | Ac | Nle-cyclo(Cys-His- | 107 | NH2 | 3.4 nM | 2.6 nM |
| | | D-Phe(4-I)-Arg-D- | | | (18%) | (16%) |
| | | Trp-Cys) | | | | |
| CTX-1236 | Ac | Arg-cyclo(Cys-D- | 108 | NH2 | 2.6 nM | 1.9 nM |
| | | Ala-Arg-D-Phe(4- | | | (66%) | (97%) |
| | | F)-Arg-D-Trp-Cys) | | | | |
| CTX-1237 | Ac | Arg-cyclo(Cys-D- | 109 | NH2 | 79 nM | 998 nM |
| | | Ala-Arg-D-Tyr-Arg- | | | (19%) | (61%) |
| | | D-Trp-Cys) | | | | |
| CTX-1238 | Ac | Arg-cyclo(Cys-D- | 110 | NH2 | 3.0 nM | 6.2 nM |
| | | Ala-Arg-D-Tyr(O- | | | (21%) | (79%) |
| | | Me)-Arg-D-Trp-Cys) | | | | |
| CTX-1239 | Ac | Arg-cyclo(Cys-D- | 111 | NH2 | 74 nM | 249 nM |
| | | Ala-Arg-D-Hph-Arg- | | | (7.9%) | (28%) |
| | | D-Trp-Cys) | | | | |
| CTX-1240 | Ac | Arg-cyclo(Cys-D- | 112 | NH2 | 31 nM | 76 nM |
| | | Ala-Arg-aMe-D- | | | (27%) | (81%) |
| | | Phe-Arg-D-Trp-Cys) | | | | |
| CTX-1242 | Ac | Arg-cyclo(Cys-D- | 113 | NH2 | 2.2 nM | 26 nM |
| | | Ala-Arg-D-Phe(4- | | | (2.8%) | (5.8%) |
| | | tBu)-Arg-D-Trp-Cys) | | | | |
| CTX-1243 | Ac | Arg-cyclo(Cys-D- | 114 | NH2 | 7.9 nM | 21 nM |
| | | Ala-Arg-D-Bip-Arg- | | | (7.7%) | (32%) |
| | | D-Trp-Cys) | | | | |
| CTX-1244 | Ac | Arg-cyclo(Cys-D- | 115 | NH2 | 13 nM | 109 nM |
| | | Ala-Arg-D-Trp-Arg- | | | (10%) | (42%) |
| | | D-Trp-Cys) | | | | |
| CTX-1245 | Ac | Arg-cyclo(Cys-D- | 116 | NH2 | 73 nM | 4,446 nM |
| | | Ala-Arg-D-Phe(4- | | | (6.3%) | (34%) |
| | | NH-Ac)-Arg-D-Trp-Cys) | | | | |
| CTX-1246 | Ac | Arg-cyclo(Cys-Aib- | 117 | NH2 | 53 nM | 1.8 nM |
| | | Arg-D-Phe(4-Br)- | | | (21%) | (80%) |
| | | Arg-D-Trp-Cys) | | | | |
| CTX-1247 | Ac | Arg-cyclo(Cys-D- | 118 | NH2 | 6.7 nM | 3.3 nM |
| | | Abu-Arg-D-Phe(4- | | | (13%) | (75%) |
| | | Br)-Arg-D-Trp-Cys) | | | | |
| CTX-1248 | Ac | Arg-cyclo(Cys-D- | 119 | NH2 | 2.8 nM | 2 nM |
| | | Ala-NMe-Arg-D- | | | (18%) | (47%) |
| | | Phe(4-Br)-Arg-D- | | | | |
| | | Trp-Cys) | | | | |
| CTX-1249 | Ac | Arg-cyclo(Cys-D- | 120 | NH2 | 6.1 nM | 14 nM |
| | | Ala-Pro-D-Phe(4- | | | (1.9%) | (22%) |
| | | Br)-Arg-D-Trp-Cys) | | | | |
| CTX-1250 | Ac | Arg-cyclo(Cys-D- | 121 | NH2 | 2,089 nM | 185 nM |
| | | Ala-D-Arg-D-Phe(4- | | | (10%) | (8.7%) |
| | | Br)-Arg-D-Trp-Cys) | | | | |
| CTX-1251 | Ac | Arg-cyclo(Cys-D- | 122 | NH2 | 93 nM | 25 nM |
| | | Ala-Arg-D-Phe(4- | | | (30%) | (31%) |
| | | Br)-NMe-Arg-D- | | | | |
| | | Trp-Cys) | | | | |
| CTX-1252 | Ac | Arg-cyclo(Cys-D- | 123 | NH2 | 57 nM | 885 nM |
| | | Ala-Arg-D-Phe(4- | | | (1.1%) | (4.4%) |
| | | Br)-D-Arg-D-Trp-Cys) | | | | |
| CTX-1253 | Ac | Arg-cyclo(Cys-D- | 124 | NH2 | 4.0 nM | 7.6 nM |
| | | Ala-Arg-D-Phe(4- | | | (9.2%) | (58%) |
| | | Br)-Cit-D-Trp-Cys) | | | | |
| CTX-1254 | Ac | Arg-cyclo(Cys-D- | 125 | NH2 | 9.2 nM | 4.2 nM |
| | | Ala-Arg-D-Phe(4-Br)- | | | (5.5%) | (22%) |
| | | Arg-D-Nal(2′)-Cys) | | | | |
| CTX-1255 | Ac | Arg-cyclo(Cys-D- | 126 | NH2 | 7.5 nM | 31 nM |
| | | Ala-Arg-D-Phe(4- | | | (28%) | (60%) |
| | | Br)-Arg-D-Phe-Cys) | | | | |
| CTX-1256 | Ac | Arg-cyclo(Cys-D- | 127 | NH2 | 592 nM | 7,244 nM |
| | | Ala-Arg-D-Phe(4- | | | (17%) | (29%) |
| | | Br)-Arg-D-Tic-Cys) | | | | |
| CTX-1258 | Ac | Arg-cyclo(Cys-D- | 128 | NH2 | 7.4 nM | 2.4 nM |
| | | Ala-Arg-D-Phe(4- | | | (36%) | (92%) |
| | | Br)-Arg-Phe-Cys) | | | | |
| CTX-1259 | Ac | Arg-cyclo(Cys-D- | 129 | NH2 | 146 nM | 1,250 nM |
| | | Ala-Arg-D-Phe(4- | | | (27%) | (70%) |
| | | Br)-Arg-Tic-Cys) | | | | |
| CTX-1260 | Ac | Arg-cyclo(Cys-D- | 130 | NH2 | 239 nM | 4,932 nM |
| | | Ala-Arg-D-Phe(4- | | | (13%) | (38%) |
| | | Br)-Arg-D-Ala-Cys) | | | | |
| CTX-1271 | Ac | Arg-cyclo(Cys-Aib- | 131 | NH2 | 117 nM | 72 nM |
| | | Arg-D-Phe-Cit-D- | | | (16%) | (95%) |
| | | Trp-Cys) | | | | |
| CTX-1272 | Ac | Arg-cyclo(Cys-D- | 132 | NH2 | 671 nM | 750 nM |
| | | Ala-Arg-NMe-D- | | | (0.9%) | (5.3%) |
| | | Phe-Arg-D-Trp-Cys) | | | | |
| CTX-1273 | Ac | Arg-cyclo(Cys-D- | 133 | NH2 | 4,775 nM | 7,430 nM |
| | | Ala-Arg-D-Tic-Arg- | | | (3.7%) | (32%) |
| | | D-Trp-Cys) | | | | |
| CTX-1274 | Ac | Arg-cyclo(Cys-D- | 134 | NH2 | 164 nM | 1,400 nM |
| | | Ala-Arg-D-Trp-Arg- | | | (5.4%) | (77%) |
| | | Trp-Cys) | | | | |
| CTX-1275 | Ac | Arg-cyclo(Cys-D- | 135 | NH2 | 248 nM | 906 nM |
| | | Ala-Arg-D-Trp-Arg- | | | (3.3%) | (24%) |
| | | D-Trp-Cys) | | | | |
| CTX-1277 | Ac | Arg-cyclo(Cys-D- | 136 | NH2 | 45 nM | 429 nM |
| | | Ala-Arg-D-Hph-Arg- | | | (5.2%) | (29%) |
| | | D-Trp-Cys) | | | | |
| CTX-1279 | Ac | Arg-cyclo(Cys-D- | 137 | NH2 | 43 nM | 72 nM |
| | | Ala-Arg-a-Me-D- | | | (20%) | (79%) |
| | | Phe-Arg-D-Trp-Cys) | | | | |
| CTX-1280 | Ac | Arg-cyclo(Cys-D- | 138 | NH2 | 541 nM | 438 nM |
| | | Ala-Arg-D-Dip-Arg- | | | (2.6%) | (4%) |
| | | D-Trp-Cys) | | | | |
| CTX-1282 | Ac | Arg-cyclo(Cys-D- | 139 | NH2 | 545 nM | 1,422 nM |
| | | Ala-Arg-D-Phe-D- | | | (4.2%) | (72%) |
| | | Arg-Trp-Cys) | | | | |
| CTX-1283 | Ac | Arg-cyclo(Cys-D- | 140 | NH2 | 719 nM | 5,861 nM |
| | | Ala-Arg-D-Phe-D- | | | (7.8%) | (55%) |
| | | Arg-D-Trp-Cys) | | | | |
| CTX-1600 | Ac | Arg-cyclo(Cys-Aib- | 141 | NH2 | >10,000 nM | 141.6 nM |
| | | Cit-D-Tyr(4-OMe)- | | | | (58%) |
| | | Arg-D-Trp-Cys) | | | | |
| CTX-1612 | Ac | Arg-Nle-cyclo(Cys- | 142 | NH2 | 4.5 nM | 2 nM |
| | | Arg-D-Phe(4-Br)- | | | (27%) | (25%) |
| | | Arg-Trp-Cys) | | | | |
| CTX-1613 | Ac | Arg-Nle-cyclo(Cys- | 143 | NH2 | 4 nM | 1.9 nM |
| | | Arg-D-Phe(4-Br)- | | | (35%) | (32%) |
| | | Arg-D-Trp-Cys) | | | | |
| CTX-1614 | Ac | Arg-Nle-cyclo(Cys- | 144 | NH2 | 3.2 nM | 1.7 nM |
| | | Arg-D-Tyr(4-OMe)- | | | (28%) | (41%) |
| | | Arg-Trp-Cys) | | | | |
| CTX-1615 | Ac | Arg-Nle-cyclo(Cys- | 145 | NH2 | 27 nM | 13 nM |
| | | Arg-D-Tyr(4-OMe)- | | | (55%) | (68%) |
| | | Arg-D-Trp-Cys) | | | | |
| CTX-1640 | Ac | cyclo(Cys-Gly-Arg- | 146 | NH2 | 10 nM | 3.4 nM |
| | | D-Phe(4-Br)-Arg- | | | (27%) | (100%) |
| | | Phe-Cys) | | | | |
| CTX-1641 | Ac | cyclo(Cys-Gly-Arg- | 147 | NH2 | 52 nM | 44 nM |
| | | D-Tyr(4-OMe)-Arg- | | | (22%) | (104%) |
| | | Phe-Cys) | | | | |
| CTX-2203 | Ac | Arg-cyclo(Cys-D- | 148 | NH2 | 29 nM | 12 nM |
| | | Ala-Pro-D-Nal(2′)- | | | (1%) | (1.4%) |
| | | Arg-Trp-Cys) | | | | |
| CTX-2204 | Ac | Arg-cyclo(Cys-D- | 149 | NH2 | 11 nM | 58 nM |
| | | Ala-Pro-D-Phe(4- | | | (0.2%) | (13%) |
| | | Br)-Arg-Trp-Cys) | | | | |
| CTX-2205 | Ac | Arg-cyclo(Cys-D- | 150 | NH2 | 18 nM | 8 nM |
| | | Ala-Arg-D-Phe(4- | | | (13%) | (88%) |
| | | Br)-Arg-Trp-Cys) | | | | |
| CTX-2206 | Ac | Arg-cyclo(Cys-D- | 151 | NH2 | 22 nM | 3 |
| | | Ala-His-D-Phe(4- | | | (28%) | (98%) |
| | | Br)-Arg-Trp-Cys) | | | | |

TABLE 3
MC4R selectivity versus MC3R and MC1R for exemplary MC4R agonists

| | | | | | MC4R | MC4R |
|---|---|---|---|---|---|---|
| Peptide | N-term. | | SEQ | C-term. | selectivity | selectivity |
| code | Mod. | Sequence | ID NO. | Mod. | vs MC3R | vs MC1R |
| CTX-1200 | Ac | Arg-cyclo(Cys-D- | 152 | NH2 | 4.09 | 0.69 |
| ^{(setmelanotide)} | | Ala-His-D-Phe- | | | | |
| | | Arg-Trp-Cys) | | | | |
| CTX-1202 | Chloroacetyl | Thioether- | 3 | NH2 | 76 | 0.89 |
| | | cyclo((D-Ala-His- | | | | |
| | | D-Phe-Arg-Trp-Cys) | | | | |
| CTX-1203 | Chloroacetyl | Thioether-cyclo(D- | 4 | NH2 | 161 | 2.32 |
| | | Ala-Nme-His-D-Phe- | | | | |
| | | Arg-Nme-Trp-Cys) | | | | |
| CTX-1205 | Ac | cyclo(Cys-Glu- | 5 | NH2 | 8.95 | Not tested |
| | | His-D-Phe-Arg- | | | | against MC1R |
| | | Trp-Cys) | | | | |
| CTX-1206 | Ac | cyclo(Cys-Gly- | 6 | NH2 | 23 | Not tested |
| | | His-D-Phe-Arg- | | | | against MC1R |
| | | Trp-Cys) | | | | |
| CTX-1207 | Ac | cyclo(Cys-D-Ala- | 7 | NH2 | 8.87 | Not tested |
| | | His-D-Phe-Arg- | | | | against MC1R |
| | | Trp-Cys) | | | | |
| CTX-1211 | Ac | Arg-cyclo(Cys-D- | 8 | NH2 | 5.49 | 3.12 |
| | | Ala-Arg-D-Phe- | | | | |
| | | Arg-Trp-Cys) | | | | |
| CTX-1213 | Ac | Arg-cyclo(Cys-D- | 9 | NH2 | 0.2 | Not tested |
| | | Ala-His-Phe-Arg- | | | | against MC1R |
| | | Trp-Cys) | | | | |
| CTX-1227 | Ac | Arg-Nle- | 10 | NH2 | 11 | 0.92 |
| | | cyclo(Cys-His-D- | | | | |
| | | Phe-Arg-Trp-Cys) | | | | |
| CTX-1228 | Ac | Arg-Nle- | 11 | NH2 | 8.38 | 3.16 |
| | | cyclo(Cys-Arg-D- | | | | |
| | | Phe-Arg-Trp-Cys) | | | | |
| CTX-1257 | Ac | Arg-cyclo(Cys-D- | 12 | NH2 | 0.06 | 0.02 |
| | | Ala-Arg-D-Phe- | | | | |
| | | Arg-D-Phe-Cys) | | | | |
| CTX-1262 | Ac | Arg-cyclo(Cys-D- | 13 | NH2 | 1.34 | 4.4 |
| | | Ala-His-D-Phe- | | | | |
| | | Arg-D-Trp-Cys) | | | | |
| CTX-1263 | Ac | Arg-cyclo(Cys-D- | 14 | NH2 | 1.62 | 1.73 |
| | | Ala-Arg-D-Phe- | | | | |
| | | Arg-D-Trp-Cys) | | | | |
| CTX-1264 | Ac | Arg-cyclo(Cys-D- | 15 | NH2 | 0.29 | 0.14 |
| | | Ala-Arg-D-Phe- | | | | |
| | | Arg-D-Nal(2′)-Cys) | | | | |
| CTX-1265 | Ac | Arg-cyclo(Cys-D- | 16 | NH2 | 0.04 | 0.02 |
| | | Ala-Arg-D-Phe- | | | | |
| | | Arg-D-Phe-Cys) | | | | |
| CTX-1267 | Ac | Arg-cyclo(Cys- | 17 | NH2 | 4.07 | 0.5 |
| | | Ala-Arg-D-Phe- | | | | |
| | | Arg-Trp-Cys) | | | | |
| CTX-1268 | Ac | Arg-cyclo(Cys- | 18 | NH2 | 30 | 6.59 |
| | | Ala-Arg-D-Phe- | | | | |
| | | Arg-D-Trp-Cys) | | | | |
| CTX-1269 | Ac | Arg-cyclo(Cys- | 19 | NH2 | 9.31 | 2.56 |
| | | NMe-Ala-Arg-D- | | | | |
| | | Phe-Arg-D-Trp-Cys) | | | | |
| CTX-1270 | Ac | Arg-cyclo(Cys- | 20 | NH2 | 1.88 | 2.49 |
| | | Aib-Arg-D-Phe- | | | | |
| | | Cit-Trp-Cys) | | | | |
| CTX-1278 | Ac | Arg-cyclo(Cys-D- | 21 | NH2 | 12 | 1.13 |
| | | Ala-Arg-aMe-D- | | | | |
| | | Phe-Arg-Trp-Cys) | | | | |
| CTX-1281 | Ac | Arg-cyclo(Cys-D- | 22 | NH2 | 0.29 | 0.44 |
| | | Ala-Arg-D-Phe- | | | | |
| | | Cit-D-Trp-Cys) | | | | |
| CTX-1284 | Ac | Arg-cyclo(Cys-D- | 23 | NH2 | 8.11 | 0.15 |
| | | Ala-Arg-D-Phe- | | | | |
| | | N-Me-Arg-D-Trp-Cys) | | | | |
| CTX-1285 | Ac | Arg-cyclo(Cys-D- | 24 | NH2 | 2.9 | 134 |
| | | Ala-Cit-D-Phe- | | | | |
| | | Arg-Trp-Cys) | | | | |
| CTX-1286 | Ac | Arg-cyclo(Cys-D- | 25 | NH2 | 2.16 | 3.4 |
| | | Ala-Cit-D-Phe- | | | | |
| | | Arg-D-Trp-Cys) | | | | |
| CTX-1287 | Ac | Arg-cyclo(Cys- | 26 | NH2 | 66 | 281 |
| | | Ala-Cit-D-Phe- | | | | |
| | | Arg-Trp-Cys) | | | | |
| CTX-1288 | Ac | Arg-cyclo(Cys- | 27 | NH2 | 16 | 3.75 |
| | | Ala-Cit-D-Phe- | | | | |
| | | Arg-D-Trp-Cys) | | | | |
| CTX-1289 | Ac | Arg-cyclo(Cys- | 28 | NH2 | 30 | 130 |
| | | Aib-Cit-D-Phe- | | | | |
| | | Arg-Trp-Cys) | | | | |
| CTX-1290 | Ac | Arg-cyclo(Cys- | 29 | NH2 | 9.23 | 10 |
| | | Aib-Cit-D-Phe- | | | | |
| | | Arg-D-Trp-Cys) | | | | |
| CTX-1291 | Ac | Arg-cyclo(Cys- | 30 | NH2 | 7.99 | 1.31 |
| | | Aib-His-D-Phe- | | | | |
| | | Arg-Trp-Cys) | | | | |
| CTX-1292 | Ac | Arg-cyclo(Cys- | 31 | NH2 | 21 | 2.46 |
| | | Aib-Arg-D-Phe- | | | | |
| | | Arg-Trp-Cys) | | | | |
| CTX-1293 | Ac | Arg-cyclo(Cys- | 32 | NH2 | 10 | 8.81 |
| | | Gly-Arg-D-Phe- | | | | |
| | | Arg-Trp-Cys) | | | | |
| CTX-1294 | Ac | Arg-cyclo(Cys- | 33 | NH2 | 11 | 111 |
| | | Gly-Cit-D-Phe- | | | | |
| | | Arg-Trp-Cys) | | | | |
| CTX-1295 | Ac | Arg-cyclo(Cys-D- | 34 | NH2 | 6.31 | 2.79 |
| | | Abu-His-D-Phe- | | | | |
| | | Arg-Trp-Cys) | | | | |
| CTX-1296 | Ac | Arg-cyclo(Cys- | 35 | NH2 | 22 | 11 |
| | | Aib-His-D-Phe- | | | | |
| | | Arg-D-Trp-Cys) | | | | |
| CTX-1297 | Ac | Arg-cyclo(Cys- | 36 | NH2 | >3,000 | 0.52 |
| | | Aib-Arg-D-Tyr(4- | | | | |
| | | OMe)-Arg-D-Trp-Cys) | | | | |
| CTX-1298 | Ac | Arg-cyclo(Cys- | 37 | NH2 | 0.93 | 0.12 |
| | | Aib-Arg-D-Tyr(4- | | | | |
| | | OMe)-Arg-Phe-Cys) | | | | |
| CTX-1299 | Ac | Arg-cyclo(Cys- | 38 | NH2 | >154 | 3.66 |
| | | Aib-Cit-D-Tyr(4- | | | | |
| | | OMe)-Arg-Phe-Cys) | | | | |
| CTX-1601 | Ac | Arg-cyclo(Cys- | 39 | NH2 | 2.3 | 0.93 |
| | | Aib-Arg-D-Phe- | | | | |
| | | Arg-Phe-Cys) | | | | |
| CTX-1602 | Ac | Arg-cyclo(Cys- | 40 | NH2 | 3.44 | 3.65 |
| | | Aib-Orn-D-Phe- | | | | |
| | | Arg-Phe-Cys) | | | | |
| CTX-1603 | Ac | Arg-cyclo(Cys- | 41 | NH2 | >18 | 2.46 |
| | | Aib-homo-Cit-D- | | | | |
| | | Phe-Arg-Phe-Cys) | | | | |
| CTX-1604 | Ac | Arg-cyclo(Cys- | 42 | NH2 | 0.11 | 3.66 |
| | | Aib-Pal(2′)-D- | | | | |
| | | Phe-Arg-Phe-Cys) | | | | |
| CTX-1605 | Ac | Arg-cyclo(Cys- | 43 | NH2 | >300 | 0.14 |
| | | Aib-Pal(3′)-D- | | | | |
| | | Phe-Arg-Phe-Cys) | | | | |
| CTX-1606 | Ac | Arg-cyclo(Cys- | 44 | NH2 | 0.62 | 2.99 |
| | | Aib-Pal(4′)-D- | | | | |
| | | Phe-Arg-Phe-Cys) | | | | |
| CTX-1607 | Ac | Arg-Nle- | 45 | NH2 | 8.15 | 36 |
| | | cyclo(Cys-Cit-D- | | | | |
| | | Phe-Arg-Trp-Cys) | | | | |
| CTX-1608 | Ac | Arg-Nle- | 46 | NH2 | 0.95 | 2.07 |
| | | cyclo(Cys-Cit-D- | | | | |
| | | Phe-Arg-D-Trp-Cys) | | | | |
| CTX-1609 | Ac | Arg-Nle- | 47 | NH2 | 4.72 | 4.15 |
| | | cyclo(Cys-Cit- | | | | |
| | | Phe-Arg-Trp-Cys) | | | | |
| CTX-1610 | Ac | Arg-Nle- | 48 | NH2 | 0.66 | 0.03 |
| | | cyclo(Cys-His- | | | | |
| | | Nal(1′)-Arg-Trp-Cys) | | | | |
| CTX-1611 | Ac | Arg-Nle- | 49 | NH2 | 6.61 | 0.25 |
| | | cyclo(Cys-Arg-D- | | | | |
| | | Nal(1′)-Arg-Trp-Cys) | | | | |
| CTX-1616 | Ac | Arg-Nle- | 50 | NH2 | 3.78 | 1.51 |
| | | cyclo(Cys-Arg-D- | | | | |
| | | Phe-Arg-D-Trp-Cys) | | | | |
| CTX-1617 | Ac | Arg-Nle- | 51 | NH2 | 11 | 1.75 |
| | | cyclo(Cys-Orn-D- | | | | |
| | | Phe-Arg-Trp-Cys) | | | | |
| CTX-1618 | Ac | Arg-Nle-cyclo(Cys- | 52 | NH2 | 16 | 16 |
| | | homo-Cit-D-Phe-Arg- | | | | |
| | | Trp-Cys) | | | | |
| CTX-1619 | Ac | Arg-Nle-cyclo(Cys- | 53 | NH2 | 13 | 86 |
| | | Pal(2′)-D-Phe- | | | | |
| | | Arg-Trp-Cys) | | | | |
| CTX-1620 | Ac | Arg-Nle-cyclo(Cys- | 54 | NH2 | 12 | 47 |
| | | Pal(3′)-D-Phe- | | | | |
| | | Arg-Trp-Cys) | | | | |
| CTX-1621 | Ac | Arg-Nle-cyclo(Cys- | 55 | NH2 | 13 | 82 |
| | | Pal(4′)-D-Phe- | | | | |
| | | Arg-Trp-Cys) | | | | |
| CTX-1622 | Ac | Arg-Nle- | 56 | NH2 | 0.32 | 0.39 |
| | | cyclo(Cys-Arg-D- | | | | |
| | | Phe-Arg-Phe-Cys) | | | | |
| CTX-1623 | Ac | Arg-Nle-cyclo(Cys- | 57 | NH2 | 2.29 | 1.08 |
| | | Arg-D-Phe(4-Br)- | | | | |
| | | Arg-Phe-Cys) | | | | |
| CTX-1624 | Ac | Arg-Nle-cyclo(Cys- | 58 | NH2 | 0.47 | 0.15 |
| | | Arg-D-Tyr(4-OMe)- | | | | |
| | | Arg-Phe-Cys) | | | | |
| CTX-1625 | Ac | Arg-Nle- | 59 | NH2 | 1.87 | 0.08 |
| | | cyclo(Cys-His-D- | | | | |
| | | Phe-Arg-Phe-Cys) | | | | |
| CTX-1626 | Ac | Arg-Nle- | 60 | NH2 | 1.6 | 3.05 |
| | | cyclo(Cys-Cit-D- | | | | |
| | | Phe-Arg-Phe-Cys) | | | | |
| CTX-1627 | Ac | cyclo(Cys-D-Ala- | 61 | NH2 | 2.47 | 9.33 |
| | | Arg-D-Phe-Arg- | | | | |
| | | Trp-Cys) | | | | |
| CTX-1628 | Ac | cyclo(Cys-D-Ala- | 62 | NH2 | 6.37 | 191 |
| | | Cit-D-Phe-Arg- | | | | |
| | | Trp-Cys) | | | | |
| CTX-1629 | Ac | cyclo(Cys-Gly- | 63 | NH2 | 19 | 15 |
| | | Arg-D-Phe-Arg- | | | | |
| | | Trp-Cys) | | | | |
| CTX-1630 | Ac | cyclo(Cys-Gly- | 64 | NH2 | 28 | 1816 |
| | | Cit-D-Phe-Arg- | | | | |
| | | Trp-Cys) | | | | |
| CTX-1631 | Ac | cyclo(Cys-Aib- | 65 | NH2 | 6.91 | 4.09 |
| | | His-D-Phe-Arg- | | | | |
| | | Trp-Cys) | | | | |
| CTX-1632 | Ac | cyclo(Cys-Aib- | 66 | NH2 | 36 | 3.18 |
| | | Arg-D-Phe-Arg- | | | | |
| | | Trp-Cys) | | | | |
| CTX-1633 | Ac | cyclo(Cys-Aib- | 67 | NH2 | 104 | 942 |
| | | Cit-D-Phe-Arg- | | | | |
| | | Trp-Cys) | | | | |
| CTX-1634 | Ac | cyclo(Cys-Aib- | 68 | NH2 | 6.68 | 4.02 |
| | | His-D-Phe-Arg- | | | | |
| | | Phe-Cys) | | | | |
| CTX-1635 | Ac | cyclo(Cys-Aib- | 69 | NH2 | 5.7 | 1.19 |
| | | Arg-D-Phe-Arg- | | | | |
| | | Phe-Cys) | | | | |
| CTX-1636 | Ac | cyclo(Cys-Aib- | 70 | NH2 | 9.44 | >12 |
| | | Cit-D-Phe-Arg- | | | | |
| | | Phe-Cys) | | | | |
| CTX-1637 | Ac | cyclo(Cys-Gly- | 71 | NH2 | 1.47 | 3.2 |
| | | His-D-Phe-Arg- | | | | |
| | | Phe-Cys) | | | | |
| CTX-1638 | Ac | cyclo(Cys-Gly- | 72 | NH2 | 1.66 | 1.04 |
| | | Arg-D-Phe-Arg- | | | | |
| | | Phe-Cys) | | | | |
| CTX-1639 | Ac | cyclo(Cys-Gly- | 73 | NH2 | 2.82 | 8.32 |
| | | Cit-D-Phe-Arg- | | | | |
| | | Phe-Cys) | | | | |
| CTX-1642 | Chloroacetyl | Thioether- | 74 | NH2 | 29 | 13 |
| | | cyclo(D-Ala-Arg- | | | | |
| | | D-Phe-Arg-Trp-Cys) | | | | |
| CTX-1643 | Chloroacetyl | Thioether- | 75 | NH2 | 133 | 261 |
| | | cyclo(D-Ala-Cit- | | | | |
| | | D-Phe-Arg-Trp-Cys) | | | | |
| CTX-1644 | Chloroacetyl | Thioether- | 76 | NH2 | 53 | 4.84 |
| | | cyclo(Gly-His-D- | | | | |
| | | Phe-Arg-Trp-Cys) | | | | |
| CTX-1645 | Chloroacetyl | Thioether- | 77 | NH2 | 42 | 9.04 |
| | | cyclo(Gly-Arg-D- | | | | |
| | | Phe-Arg-Trp-Cys) | | | | |
| CTX-1646 | Chloroacetyl | Thioether- | 78 | NH2 | 193 | >770 |
| | | cyclo(Gly-Cit-D- | | | | |
| | | Phe-Arg-Trp-Cys) | | | | |
| CTX-1647 | Chloroacetyl | Thioether- | 79 | NH2 | 30 | 3.66 |
| | | cyclo(Aib-His-D- | | | | |
| | | Phe-Arg-Trp-Cys) | | | | |
| CTX-1648 | Chloroacetyl | Thioether- | 80 | NH2 | 63 | 13 |
| | | cyclo(Aib-Arg-D- | | | | |
| | | Phe-Arg-Trp-Cys) | | | | |
| CTX-1649 | Chloroacetyl | Thioether- | 81 | NH2 | 72 | 1652 |
| | | cyclo(Aib-Cit-D- | | | | |
| | | Phe-Arg-Trp-Cys) | | | | |
| CTX-1650 | Chloroacetyl | Thioether- | 82 | NH2 | 53 | 0.95 |
| | | cyclo(Aib-His-D- | | | | |
| | | Phe-Arg-Phe-Cys) | | | | |
| CTX-1651 | Chloroacetyl | Thioether- | 83 | NH2 | >8 | 1.45 |
| | | cyclo(Aib-Arg-D- | | | | |
| | | Phe-Arg-Phe-Cys) | | | | |
| CTX-1652 | Chloroacetyl | Thioether- | 84 | NH2 | Not active | Not active |
| | | cyclo(Aib-Cit-D- | | | | |
| | | Phe-Arg-Phe-Cys) | | | | |
| CTX-1653 | Chloroacetyl | Thioether- | 85 | NH2 | >8 | 0.83 |
| | | cyclo(Gly-His-D- | | | | |
| | | Phe-Arg-Phe-Cys) | | | | |
| CTX-1654 | Chloroacetyl | Thioether- | 86 | NH2 | 5.41 | 0.62 |
| | | cyclo(Gly-Arg-D- | | | | |
| | | Phe-Arg-Phe-Cys) | | | | |
| CTX-1655 | Chloroacetyl | Thioether- | 87 | NH2 | Not active | Not active |
| | | cyclo(Gly-Cit-D- | | | | |
| | | Phe-Arg-Phe-Cys) | | | | |
| CTX-1656 | Chloroacetyl | Thioether- | 88 | NH2 | 1.64 | 1 |
| | | cyclo(D-Ala-His- | | | | |
| | | D-Phe-Arg-Phe-Cys) | | | | |
| CTX-1657 | Chloroacetyl | Thioether- | 89 | NH2 | 3.56 | 1.42 |
| | | cyclo(D-Ala-Arg- | | | | |
| | | D-Phe-Arg-Phe-Cys) | | | | |
| CTX-1658 | Chloroacetyl | Thioether- | 90 | NH2 | Not active | Not active |
| | | cyclo(D-Ala-Cit- | | | | |
| | | D-Phe-Arg-Phe-Cys) | | | | |
| CTX-2128 | Ac | Nle-cyclo(Cys-His- | 91 | NH2 | 0.01 | Not tested |
| | | Nal(2′)-Arg-D-Trp- | | | | against MC1R |
| | | Cys)-Arg-Phe-Gly | | | | |

TABLE 4
MC4R selectivity versus MC3R and MC1R for exemplary MC4R agonists
with significant MC3R antagonism or partial agonism

| | | | | | MC4R | MC4R |
|---|---|---|---|---|---|---|
| Peptide | N-term. | | SEQ | C-term. | selectivity | selectivity |
| code | Mod. | Sequence | ID NO. | Mod. | vs MC3R | vs MC1R |
| CTX-1200 | Ac | Arg-cyclo(Cys-D- | 152 | NH2 | 4.09 | 0.69 |
| ^{(setmelanotide)} | | Ala-His-D-Phe-Arg- | | | | |
| | | Trp-Cys) | | | | |
| CTX-1212 | Ac | Arg-cyclo(Cys-D- | 92 | NH2 | 0.07 | 0.07 |
| | | Ala-Arg-Phe(4-Br)- | | | | |
| | | Arg-Trp-Cys) | | | | |
| CTX-1214 | Ac | Arg-cyclo(Cys-D- | 93 | NH2 | 0.2 | Not tested |
| | | Ala-Arg-D-Phe(4- | | | | against MC1R |
| | | Br)-Arg-D-Trp-Cys) | | | | |
| CTX-1215 | Ac | Arg-cyclo(Cys-D- | 94 | NH2 | 0.05 | 0.07 |
| | | Ala-Arg-Phe(4-Br)- | | | | |
| | | Arg-D-Trp-Cys) | | | | |
| CTX-1216 | Ac | Arg-cyclo(Cys-D-Ala- | 95 | NH2 | 1.99 | 0.82 |
| | | Arg-D-Phe(4-Br)-Arg-D- | | | | |
| | | Trp-Cys)-Lys-Pro-Val | | | | |
| CTX-1217 | Ac | Arg-cyclo(Cys-D-Ala- | 96 | NH2 | 2.73 | 1.12 |
| | | Arg-D-Phe(4-Br)-Arg- | | | | |
| | | D-Trp-Cys)-Lys-Pro | | | | |
| CTX-1218 | Ac | Arg-cyclo(Cys-D- | 97 | NH2 | 1.85 | 1 |
| | | Ala-Arg-D-Phe(4-Br)- | | | | |
| | | Arg-D-Trp-Cys)-Lys | | | | |
| CTX-1219 | Ac | Arg-cyclo(Cys-D-Ala- | 98 | free | 1.99 | 1 |
| | | Arg-D-Phe(4-Br)-Arg-D- | | | | |
| | | Trp-Cys)-Arg-Phe-Gly | | | | |
| CTX-1220 | Ac | Arg-cyclo(Cys-D-Ala- | 99 | NH2 | 3.82 | 1.53 |
| | | Arg-D-Phe(4-Br)-Arg- | | | | |
| | | D-Trp-Cys)-Arg-Phe | | | | |
| CTX-1222 | Ac | Arg-cyclo(Cys-D-Ala- | 100 | NH2 | 0.33 | 0.1 |
| | | Arg-D-Phe(4-Br)-Arg- | | | | |
| | | D-Trp-Cys)-Arg-D-Phe | | | | |
| CTX-1226 | R2 | Arg-cyclo(Cys-D- | 101 | NH2 | 3.21 | 1.33 |
| | | Ala-Arg-D-Phe(4- | | | | |
| | | Br)-Arg-D-Trp-Cys) | | | | |
| CTX-1229 | Ac | Arg-Nle-cyclo(Cys- | 102 | NH2 | 3.5 nM | 18 nM |
| | | His-D-Phe(4-Br)- | | | (16%) | (31%) |
| | | Arg-Trp-Cys) | | | | |
| CTX-1230 | Ac | Arg-Nle-cyclo(Cys- | 103 | NH2 | 1.95 | 0.18 |
| | | Arg-D-Phe(4-Br)- | | | | |
| | | Arg-Trp-Cys) | | | | |
| CTX-1231 | Ac | Arg-Nle-cyclo(Cys- | 104 | NH2 | 3.04 | 0.19 |
| | | His-D-Phe(4-Br)- | | | | |
| | | Arg-D-Trp-Cys) | | | | |
| CTX-1232 | Ac | Arg-Nle-cyclo(Cys- | 105 | NH2 | 2.44 | 0.39 |
| | | Arg-D-Phe(4-Br)- | | | | |
| | | Arg-D-Trp-Cys) | | | | |
| CTX-1233 | Ac | Arg-Nle-cyclo(Cys- | 106 | NH2 | 1.88 | 0.26 |
| | | His-D-Phe(4-I)-Arg- | | | | |
| | | D-Trp-Cys) | | | | |
| CTX-1234 | Ac | Nle-cyclo(Cys-His- | 107 | NH2 | 1.32 | 0.2 |
| | | D-Phe(4-I)-Arg-D- | | | | |
| | | Trp-Cys) | | | | |
| CTX-1236 | Ac | Arg-cyclo(Cys-D- | 108 | NH2 | 1.36 | 2.01 |
| | | Ala-Arg-D-Phe(4- | | | | |
| | | F)-Arg-D-Trp-Cys) | | | | |
| CTX-1237 | Ac | Arg-cyclo(Cys-D- | 109 | NH2 | 0.08 | 0.24 |
| | | Ala-Arg-D-Tyr-Arg- | | | | |
| | | D-Trp-Cys) | | | | |
| CTX-1238 | Ac | Arg-cyclo(Cys-D- | 110 | NH2 | 0.49 | 0.44 |
| | | Ala-Arg-D-Tyr(O- | | | | |
| | | Me)-Arg-D-Trp-Cys) | | | | |
| CTX-1239 | Ac | Arg-cyclo(Cys-D- | 111 | NH2 | 0.3 | 0.1 |
| | | Ala-Arg-D-Hph-Arg- | | | | |
| | | D-Trp-Cys) | | | | |
| CTX-1240 | Ac | Arg-cyclo(Cys-D- | 112 | NH2 | 0.41 | 0.46 |
| | | Ala-Arg-aMe-D- | | | | |
| | | Phe-Arg-D-Trp-Cys) | | | | |
| CTX-1242 | Ac | Arg-cyclo(Cys-D- | 113 | NH2 | 0.08 | 0.64 |
| | | Ala-Arg-D-Phe(4- | | | | |
| | | tBu)-Arg-D-Trp-Cys) | | | | |
| CTX-1243 | Ac | Arg-cyclo(Cys-D- | 114 | NH2 | 0.38 | 0.21 |
| | | Ala-Arg-D-Bip-Arg- | | | | |
| | | D-Trp-Cys) | | | | |
| CTX-1244 | Ac | Arg-cyclo(Cys-D- | 115 | NH2 | 0.11 | 0.26 |
| | | Ala-Arg-D-Trp-Arg- | | | | |
| | | D-Trp-Cys) | | | | |
| CTX-1245 | Ac | Arg-cyclo(Cys-D- | 116 | NH2 | 0.02 | 0.08 |
| | | Ala-Arg-D-Phe(4-NH- | | | | |
| | | Ac)-Arg-D-Trp-Cys) | | | | |
| CTX-1246 | Ac | Arg-cyclo(Cys-Aib- | 117 | NH2 | 2.91 | 0.74 |
| | | Arg-D-Phe(4-Br)- | | | | |
| | | Arg-D-Trp-Cys) | | | | |
| CTX-1247 | Ac | Arg-cyclo(Cys-D- | 118 | NH2 | 2.05 | 0.43 |
| | | Abu-Arg-D-Phe(4- | | | | |
| | | Br)-Arg-D-Trp-Cys) | | | | |
| CTX-1248 | Ac | Arg-cyclo(Cys-D-Ala- | 119 | NH2 | 1.36 | 1.31 |
| | | NMe-Arg-D-Phe(4-Br)- | | | | |
| | | Arg-D-Trp-Cys) | | | | |
| CTX-1249 | Ac | Arg-cyclo(Cys-D- | 120 | NH2 | 0.45 | 5.08 |
| | | Ala-Pro-D-Phe(4- | | | | |
| | | Br)-Arg-D-Trp-Cys) | | | | |
| CTX-1250 | Ac | Arg-cyclo(Cys-D- | 121 | NH2 | 11 | 1.06 |
| | | Ala-D-Arg-D-Phe(4- | | | | |
| | | Br)-Arg-D-Trp-Cys) | | | | |
| CTX-1251 | Ac | Arg-cyclo(Cys-D-Ala- | 122 | NH2 | 3.73 | 0.11 |
| | | Arg-D-Phe(4-Br)-NMe- | | | | |
| | | Arg-D-Trp-Cys) | | | | |
| CTX-1252 | Ac | Arg-cyclo(Cys-D- | 123 | NH2 | 0.06 | 0.04 |
| | | Ala-Arg-D-Phe(4- | | | | |
| | | Br)-D-Arg-D-Trp-Cys) | | | | |
| CTX-1253 | Ac | Arg-cyclo(Cys-D- | 124 | NH2 | 0.53 | 0.57 |
| | | Ala-Arg-D-Phe(4- | | | | |
| | | Br)-Cit-D-Trp-Cys) | | | | |
| CTX-1254 | Ac | Arg-cyclo(Cys-D-Ala- | 125 | NH2 | 2.19 | 0.26 |
| | | Arg-D-Phe(4-Br)-Arg- | | | | |
| | | D-Nal(2′)-Cys) | | | | |
| CTX-1255 | Ac | Arg-cyclo(Cys-D- | 126 | NH2 | 0.24 | 0.05 |
| | | Ala-Arg-D-Phe(4- | | | | |
| | | Br)-Arg-D-Phe-Cys) | | | | |
| CTX-1256 | Ac | Arg-cyclo(Cys-D- | 127 | NH2 | 0.08 | 0.003 |
| | | Ala-Arg-D-Phe(4- | | | | |
| | | Br)-Arg-D-Tic-Cys) | | | | |
| CTX-1258 | Ac | Arg-cyclo(Cys-D- | 128 | NH2 | 3.04 | 0.53 |
| | | Ala-Arg-D-Phe(4- | | | | |
| | | Br)-Arg-Phe-Cys) | | | | |
| CTX-1259 | Ac | Arg-cyclo(Cys-D- | 129 | NH2 | 0.12 | 0.01 |
| | | Ala-Arg-D-Phe(4- | | | | |
| | | Br)-Arg-Tic-Cys) | | | | |
| CTX-1260 | Ac | Arg-cyclo(Cys-D- | 130 | NH2 | 0.05 | 0.01 |
| | | Ala-Arg-D-Phe(4- | | | | |
| | | Br)-Arg-D-Ala-Cys) | | | | |
| CTX-1271 | Ac | Arg-cyclo(Cys-Aib- | 131 | NH2 | 1.63 | 2.9 |
| | | Arg-D-Phe-Cit-D- | | | | |
| | | Trp-Cys) | | | | |
| CTX-1272 | Ac | Arg-cyclo(Cys-D- | 132 | NH2 | 0.9 | 0.21 |
| | | Ala-Arg-NMe-D- | | | | |
| | | Phe-Arg-D-Trp-Cys) | | | | |
| CTX-1273 | Ac | Arg-cyclo(Cys-D- | 133 | NH2 | 0.64 | 0.05 |
| | | Ala-Arg-D-Tic-Arg- | | | | |
| | | D-Trp-Cys) | | | | |
| CTX-1274 | Ac | Arg-cyclo(Cys-D- | 134 | NH2 | 0.12 | 0.19 |
| | | Ala-Arg-D-Trp-Arg- | | | | |
| | | Trp-Cys) | | | | |
| CTX-1275 | Ac | Arg-cyclo(Cys-D- | 135 | NH2 | 0.27 | 0.22 |
| | | Ala-Arg-D-Trp-Arg- | | | | |
| | | D-Trp-Cys) | | | | |
| CTX-1277 | Ac | Arg-cyclo(Cys-D- | 136 | NH2 | 0.1 | 0.05 |
| | | Ala-Arg-D-Hph-Arg- | | | | |
| | | D-Trp-Cys) | | | | |
| CTX-1279 | Ac | Arg-cyclo(Cys-D- | 137 | NH2 | 0.61 | 0.83 |
| | | Ala-Arg-a-Me-D- | | | | |
| | | Phe-Arg-D-Trp-Cys) | | | | |
| CTX-1280 | Ac | Arg-cyclo(Cys-D- | 138 | NH2 | 1.24 | 0.1 |
| | | Ala-Arg-D-Dip-Arg- | | | | |
| | | D-Trp-Cys) | | | | |
| CTX-1282 | Ac | Arg-cyclo(Cys-D- | 139 | NH2 | 0.38 | 0.14 |
| | | Ala-Arg-D-Phe-D- | | | | |
| | | Arg-Trp-Cys) | | | | |
| CTX-1283 | Ac | Arg-cyclo(Cys-D- | 140 | NH2 | 0.12 | 0.03 |
| | | Ala-Arg-D-Phe-D- | | | | |
| | | Arg-D-Trp-Cys) | | | | |
| CTX-1600 | Ac | Arg-cyclo(Cys-Aib- | 141 | NH2 | >70 | >70 |
| | | Cit-D-Tyr(4-OMe)- | | | | |
| | | Arg-D-Trp-Cys) | | | | |
| CTX-1612 | Ac | Arg-Nle-cyclo(Cys- | 142 | NH2 | 2.27 | 0.09 |
| | | Arg-D-Phe(4-Br)- | | | | |
| | | Arg-Trp-Cys) | | | | |
| CTX-1613 | Ac | Arg-Nle-cyclo(Cys- | 143 | NH2 | 2.1 | 0.79 |
| | | Arg-D-Phe(4-Br)- | | | | |
| | | Arg-D-Trp-Cys) | | | | |
| CTX-1614 | Ac | Arg-Nle-cyclo(Cys- | 144 | NH2 | 1.89 | 0.08 |
| | | Arg-D-Tyr(4-OMe)- | | | | |
| | | Arg-Trp-Cys) | | | | |
| CTX-1615 | Ac | Arg-Nle-cyclo(Cys- | 145 | NH2 | 2.14 | 0.26 |
| | | Arg-D-Tyr(4-OMe)- | | | | |
| | | Arg-D-Trp-Cys) | | | | |
| CTX-1640 | Ac | cyclo(Cys-Gly-Arg- | 146 | NH2 | 3.09 | 3.72 |
| | | D-Phe(4-Br)-Arg- | | | | |
| | | Phe-Cys) | | | | |
| CTX-1641 | Ac | cyclo(Cys-Gly-Arg- | 147 | NH2 | 1.18 | 0.28 |
| | | D-Tyr(4-OMe)-Arg- | | | | |
| | | Phe-Cys) | | | | |
| CTX-2203 | Ac | Arg-cyclo(Cys-D- | 148 | NH2 | 2.45 | Not tested |
| | | Ala-Pro-D-Nal(2′)- | | | | against MC1R |
| | | Arg-Trp-Cys) | | | | |
| CTX-2204 | Ac | Arg-cyclo(Cys-D- | 149 | NH2 | 0.19 | Not tested |
| | | Ala-Pro-D-Phe(4- | | | | against MC1R |
| | | Br)-Arg-Trp-Cys) | | | | |
| CTX-2205 | Ac | Arg-cyclo(Cys-D- | 150 | NH2 | 2.12 | 0.2 |
| | | Ala-Arg-D-Phe(4- | | | | |
| | | Br)-Arg-Trp-Cys) | | | | |
| CTX-2206 | Ac | Arg-cyclo(Cys-D- | 151 | NH2 | 7.35 | Not tested |
| | | Ala-His-D-Phe(4- | | | | against MC1R |
| | | Br)-Arg-Trp-Cys) | | | | |

### Example 2

### Plasma Stability

Pooled mouse plasma was prepared and stored at −80° C, prior to use, 396 μL mouse plasma was incubated at 37° C. for 5 minutes in 1.5 mL microcentrifuge tubes. 4 μL of 100 μM test or control compound/peptide was added to each tube and incubated for 0.5, 15, 30, 60, 120, or 240 minutes. An aliquot of 40 μL of each reaction was stopped by the addition of 4 volume of cold acetonitrile containing 200 ng/mL of The incubation solution was centrifuged at 3500 rpm for 10 minutes to precipitate protein. The supernatant was used for LC/MS/MS analysis. The natural log peak area ratio (compound peak area/internal standard peak area) was plotted against time and the gradient of the line determined. Data for exemplars are shown below.

### LC-MS/MS Method:

| | | Gradient Program: | | |
|---|---|---|---|---|
| Chromatographic Conditions: | | Time | % A | % B |
| Column: | Agilent Poroshell 120 SB-C18, | 0.01 | 70 | 30 |
| | 2.7 μm | 0.80 | 70 | 30 |
| Mobile Phase | 0.1% formic acid in purified | 1.00 | 5 | 95 |
| A: | deionized water | 3.00 | 5 | 95 |
| Mobile Phase | 0.1% formic acid in acetonitrile | 3.10 | 99 | 1 |
| B: | | 5.10 | 99 | 1 |
| Flow Rate: | 0.3 mL/min | | | |
| Injection | 5 μL | | | |
| Volume: | | | | |
| Run Time: | 5.5 min | | | |

### MS/MS Conditions in 4500

MRM-transitions:

| | Q1 Mass | | | | | |
|---|---|---|---|---|---|---|
| | (M + H⁺, | Q3 Mass | | | | |
| Compounds | m/z) | (m/z) | DP | EP | CE | CXP |
| CTX1200 | 373.2 | 287.1 | 87.98 | 12.14 | 33.93 | 10.10 |
| CTX1211 | 379.5 | 287.2 | 97.85 | 11.99 | 34.09 | 11.11 |
| CTX1227 | 387.1 | 287.0 | 89.83 | 12.29 | 31.25 | 12.80 |
| CTX1228 | 393.5 | 287.1 | 88.98 | 12.18 | 34.92 | 10.91 |
| DTrp8-g-MSH | 524.5 | 654.7 | 90.00 | 3.10 | 24.30 | 8.10 |
| (IS) | | | | | | |
| Procainamide | 235.8 | 119.9 | 67.00 | 7.12 | 26.90 | 17.93 |
| Procaine | 236.9 | 164.1 | 105.8 | 4.05 | 14.40 | 1.39 |

### Results

### Mouse Plasma Stability

The mouse plasma stability and T_{1/2} of test compounds are listed in the Table and plotted in the graph below.

### Example 3

### Plasma Protein Binding Data

Solutions of test compound in DMSO were prepared at 1 mM, 100 uM and 10 uM, 5 ul of each solution of test compound was added to 495 uL plasma, and incubated at 25° C. for 5 min. Sample was added to a Centrifree® device, then centrifuged at 2000× g for 20 min to obtain desired filtrate volume. Initial plasma samples and ultrafiltrates were subjected to LC-MS/MS. % Free peptide=(concentration of ultrafiltrates/concentration of plasma)×100%. % Bound=100%−% Free

TABLE 6
Peptide Stability and Plasma Protein Binding Data

| | | Protein | Protein | Protein |
|---|---|---|---|---|
| | Plasma Half- | Binding | Binding | Binding |
| Compound | life (min) | 0.1 uM (%) | 1.0 uM (%) | 10.0 uM (%) |
| setmelanotide | >180 | 43 | 57 | 61 |
| CTX-1211 | >180 | 51 | 63 | 61 |
| CTX-1227 | 63 | 50 | 47 | 54 |
| CTX-1228 | 55 | 37 | 39 | 39 |
| CTX-1236 | 227 | 45 | 49 | 51 |
| CTX-1238 | 172 | 53 | 47 | 41 |
| CTX-1246 | >240 | 73 | 44 | 43 |
| CTX-1247 | 199 | 49 | 53 | 53 |
| CTX-1258 | >240 | 47 | 50 | 50 |
| CTX-1268 | 105 | 52 | 58 | 62 |
| CTX-1285 | >240 | 45 | 39 | 41 |
| CTX-1258 | >240 | 47 | 50 | 50 |

### Example 4

### Pharmacokinetics

The drug at 1.5 mg/mL in PBS containing 10% DMSO and 10% PEG-400 was given by IP injection (15 mg/kg). At the given time points (0.5 h, 2 h, 4 h, 7 h and 24 h), brain samples were taken out and frozen at −80° C. immediately for later preparation and analysis, and blood samples were collected using heparinized calibrated pipettes. Blood samples were centrifuged at 15000 rpm for 10 min. Subsequently, blood plasma was collected from the upper layer. The plasma was frozen at −80° C. for later analysis.

TABLE 7
Volume of CTX-1227 Dose Solution.

| | | | | Animal | Compound | Compound | Sample | |
|---|---|---|---|---|---|---|---|---|
| | | | Dose | Weight | Volume | Concentration | time | Brain |
| Compound | Route | Mouse | (mg/kg) | (g) | (μL) | (mg/ml) | (h) | weight(g) |
| CTX-1227 | IP | 1 | 15 | 25.8 | 258.0 | 1.5 | 0.5 | 0.429 |
| CTX-1227 | IP | 2 | 15 | 29.8 | 298.0 | 1.5 | 0.5 | 0.479 |
| CTX-1227 | IP | 3 | 15 | 28.8 | 288.0 | 1.5 | 0.5 | 0.439 |
| CTX-1227 | IP | 4 | 15 | 30.8 | 308.0 | 1.5 | 2 | 0.430 |
| CTX-1227 | IP | 5 | 15 | 28.8 | 288.0 | 1.5 | 2 | 0.447 |
| CTX-1227 | IP | 6 | 15 | 26.8 | 268.0 | 1.5 | 2 | 0.444 |
| CTX-1227 | IP | 7 | 15 | 28.0 | 280.0 | 1.5 | 4 | 0.482 |
| CTX-1227 | IP | 8 | 15 | 27.4 | 274.0 | 1.5 | 4 | 0.437 |
| CTX-1227 | IP | 9 | 15 | 34.1 | 341.0 | 1.5 | 4 | 0.459 |
| CTX-1227 | IP | 10 | 15 | 27.8 | 278.0 | 1.5 | 7 | 0.436 |
| CTX-1227 | IP | 11 | 15 | 29.5 | 295.0 | 1.5 | 7 | 0.482 |
| CTX-1227 | IP | 12 | 15 | 26.5 | 265.0 | 1.5 | 7 | 0.425 |
| CTX-1227 | IP | 13 | 15 | 29.6 | 296.0 | 1.5 | 24 | 0.425 |
| CTX-1227 | IP | 14 | 15 | 29.4 | 294.0 | 1.5 | 24 | 0.306 |
| CTX-1227 | IP | 15 | 15 | 27.4 | 274.0 | 1.5 | 24 | 0.383 |

TABLE 8
Volume of setmelanotide Dose Solution.

| | | | | Animal | Compound | Compound | Sample | |
|---|---|---|---|---|---|---|---|---|
| | | | Dose | Weight | Volume | Concentration | time | Brain |
| Compound | Route | Mouse | (mg/kg) | (g) | (μL) | (mg/ml) | (h) | weight(g) |
| setmelanotide | IP | 1 | 15 | 26.6 | 266.0 | 1.5 | 0.5 | 0.456 |
| setmelanotide | IP | 2 | 15 | 27.3 | 273.0 | 1.5 | 0.5 | 0.464 |
| setmelanotide | IP | 3 | 15 | 26.5 | 265.0 | 1.5 | 0.5 | 0.456 |
| setmelanotide | IP | 4 | 15 | 28.8 | 288.0 | 1.5 | 2 | 0.486 |
| setmelanotide | IP | 5 | 15 | 30.0 | 300,0 | 1.5 | 2 | 0.466 |
| setmelanotide | IP | 6 | 15 | 31.1 | 311.0 | 1.5 | 2 | 0.480 |
| setmelanotide | IP | 7 | 15 | 34.4 | 344.0 | 1.5 | 4 | 0.491 |
| setmelanotide | IP | 8 | 15 | 27.5 | 275.0 | 1.5 | 4 | 0.455 |
| setmelanotide | IP | 9 | 15 | 29.2 | 292.0 | 1.5 | 4 | 0.449 |
| setmelanotide | IP | 10 | 15 | 25.9 | 259.0 | 1.5 | 7 | 0.473 |
| setmelanotide | IP | 11 | 15 | 31.8 | 318.0 | 1.5 | 7 | 0.485 |
| setmelanotide | IP | 12 | 15 | 28.8 | 288.0 | 1.5 | 7 | 0.484 |
| setmelanotide | IP | 13 | 15 | 27.8 | 278.0 | 1.5 | 24 | 0.444 |
| setmelanotide | IP | 14 | 15 | 28.5 | 285.0 | 1.5 | 24 | 0.485 |
| setmelanotide | IP | 15 | 15 | 30.0 | 300.0 | 1.5 | 24 | 0.469 |

### 1.1 Specificity

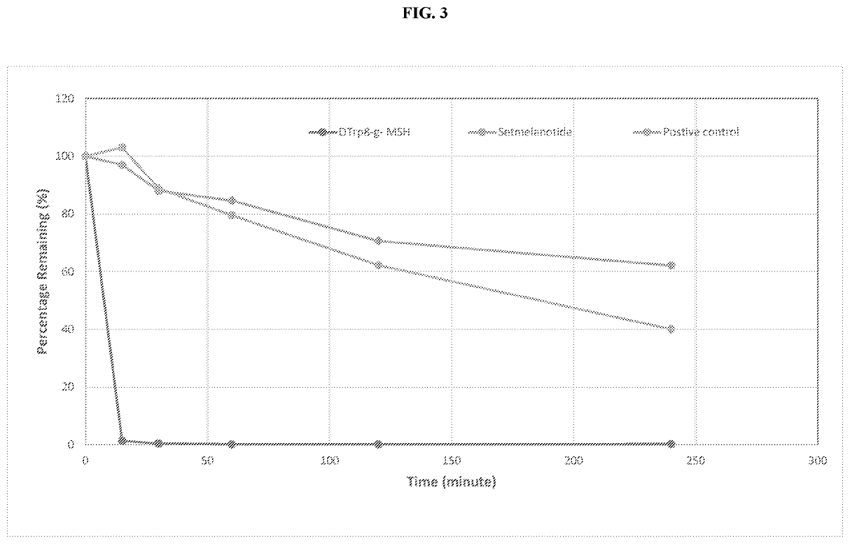

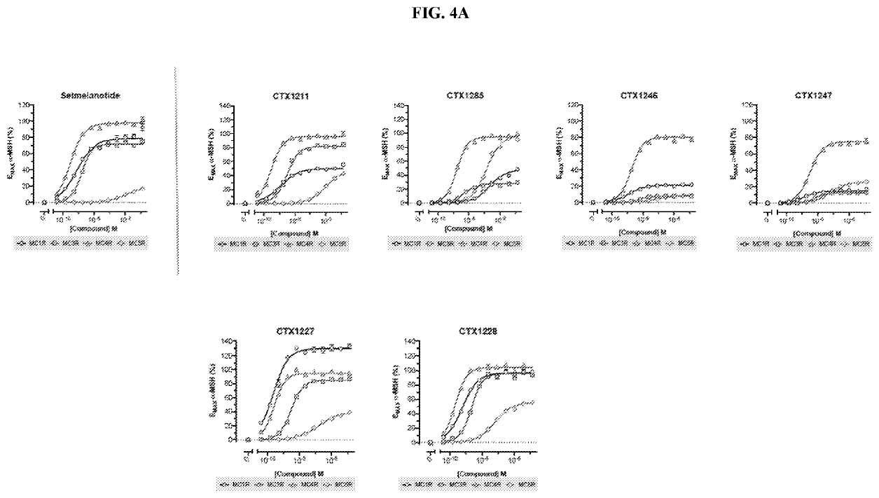

The chromatographic conditions showed that the Plasma, Brain, internal standard (CTX-1227 and setmelanotide are internal standard compounds for each other) have no interference to the CTX-1227 and setmelanotide determination. (FIG. 3-4).

### 1.2 Calibration Curves

The analytical curves were constructed using non-zero standards in the blank Plasma and Brain Samples. A blank sample (matrix sample processed without internal standard) was used to exclude contamination. The linear regression analysis of CTX1227/setmelanotide were performed by plotting the peak area ratio (y) against the CTX1227/setmelanotide concentration (x) in ng/mL, respectively. The linearity of the relationship between peak area ratio and concentration was demonstrated by the correlation coefficients (R).

### 1.3 LC-MS/MS Conditions

TABLE 9
Chromatographic Conditions.

| | | Gradient Program: | | |
|---|---|---|---|---|
| Chromatographic Conditions: | | Time | % A | % B |
| Column: | Agilent Poroshell 120 SB-C18, 2.7 μm | 0.01 | 70 | 30 |
| Mobile Phase | 0.1% formic acid in purified deionized | 0.80 | 70 | 30 |
| A: | water | 1.00 | 5 | 95 |
| Mobile Phase | 0.1% formic acid in acetonitrile | 3.00 | 5 | 95 |
| B: | | 3.10 | 99 | 1 |
| Flow Rate: | 0.5 mL/min | 5.10 | 99 | 1 |
| Injection | 20 μL | | | |
| Volume: | | | | |
| Run Time: | 5.5 min | | | |

### 1.4 MS/MS Conditions

TABLE 10
MRM-transitions with 5 ms pause between masses.

| | Q1 Mass | | | | | |
|---|---|---|---|---|---|---|
| | (M + H⁺, | Q3 Mass | | | | |
| Compounds | m/z) | (m/z) | DP | EP | CE | CXP |
| CTX1227 | 387.1 | 287.0 | 89.83 | 12.29 | 31.25 | 12.80 |
| setmelanotide | 373.2 | 287.1 | 87.98 | 12.14 | 33.93 | 10.10 |

### 2 Result

TABLE11
CTX-1227 and setmelanotide concentration in mice Plasma and Brain.

| | Dose IP 15 mg/kg | | | | | | | | | |
|---|---|---|---|---|---|---|---|---|---|---|
| Time point | Plasma (ng/mL) | | | | | Brain (ng/g) | | | | |
| (Hours) | 1 | 2 | 3 | Mean | SD | 1 | 2 | 3 | Mean | SD |

CTX1227

| | | | | | | | | | | |
|---|---|---|---|---|---|---|---|---|---|---|
| 0.5 | 20900 | 25000 | 68700 | 38200.00 | 26493.21 | 99 | 263.5 | 182 | 181.50 | 82.25 |
| 2 | 50200 | 17300 | 20700 | 29400.00 | 18093.37 | 287.5 | 176.5 | 327.5 | 263.83 | 78.23 |
| 4 | 19400 | 23300 | 33400 | 25366.67 | 7225.19 | 158 | 142.5 | 210 | 170.17 | 35.36 |
| 7 | 9360 | 9210 | 2880 | 7150.00 | 3698.69 | 223 | 224.5 | 119 | 188.83 | 60.48 |
| 24 | 15 | BLQ | BLQ | N/A | N/A | BlQ | BLQ | BLQ | N/A | N/A |

setmelanotide

| | | | | | | | | | | |
|---|---|---|---|---|---|---|---|---|---|---|
| 0.5 | 67000 | 37200 | 25800 | 43333.33 | 21273.77 | 306.5 | 231.5 | 247.5 | 261.83 | 39.50 |
| 2 | 7190 | 11000 | 4580 | 7590.00 | 3228.64 | 207.5 | 121.5 | 220.5 | 183.17 | 53.80 |
| 4 | 455 | 9590 | 17.8 | 3354.27 | 5404.73 | 105.5 | 279.5 | BLQ | N/A | N/A |
| 7 | 64.2 | 69.3 | 55.3 | 62.93 | 7.09 | BlQ | BLQ | BLQ | N/A | N/A |
| 24 | 8.04 | 7.37 | 8.81 | 8.07 | 0.72 | BlQ | BLQ | BLQ | N/A | N/A |

N/A: No Data.
BLQ: Below Limit of Quantification

### Example 5

### Acute Feeding Studies

### Mice

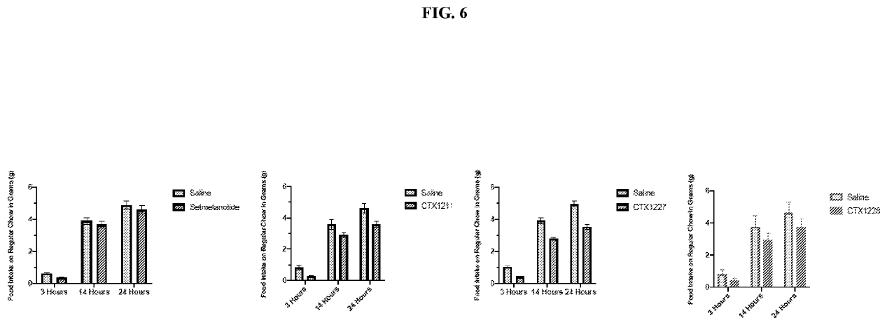

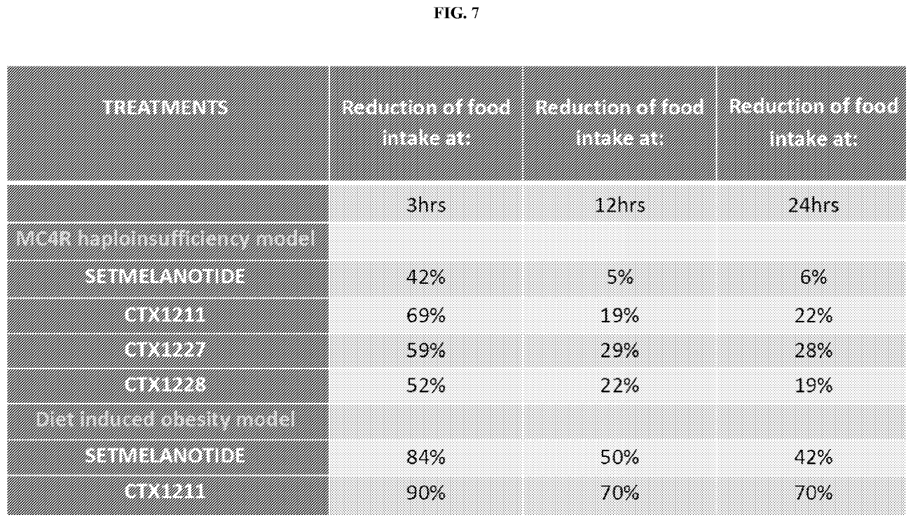

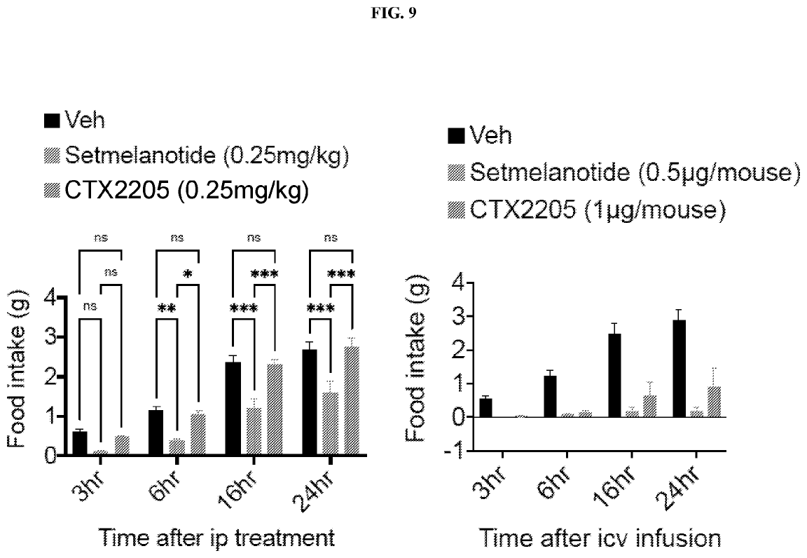

Studies utilized 7 MC4R+/− male C57BL/6J mice per condition, 8-24 weeks of age (FIG. 6), or 8-24 weeks of age WT male C57BL/6J mice made obese by 16-20 weeks of high fat diet, beginning at 8 weeks of age (diet induced obese mice, FIG. 7; all mice, FIG. 9). Mice are individually housed.

### Acclimatization:

Animals are injected daily at 5 PM with 150 ml of saline for 3 days or until animals acclimate, as indicated by the return of 14 hr food intake to pre-treatment levels. Animals are then randomized, and injected With either vehicle or drug in vehicle on the experimental day.

### I.P. Injection Protocol:

Inject 150 μl of Saline (0.9% Nacl) or compound (already prepared at 2.5 mg/kg dose) and aliquoted and stored at −80C).
Remove the needle from the vial and flick the needle to get the air bubbles out.
Inject to the right or left side of the midline, into the abdominal cavity.
Injection at 5 pm, 1 hour before the starting of dark cycle. Record body weight and food weight at 8 pm (3 hours), 7am (14 hours) and 5 pm next day (24 hours).

### ICV Injection Protocol

For the intracerebroventricular (icv) cannulation, DIO mice were implanted with a stainless steel cannula into the right lateral ventricle under isoflurane anesthesia at the following coordinates; L; 0.460, AP; −1.0, DV; −2.20 with respect to the bregma. Following recovery, mice were tested for positive cannulation with 20 ng of Angiotensin II. Mice were infused with vehicle (10% DMSO in water), or peptides indicated into the lateral ventricle within 30 min of the onset of dark cycle.

### Example 6

### Low Dose Infusion Study

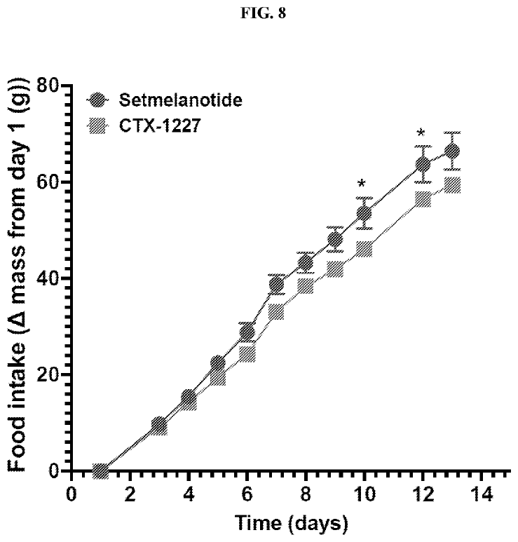

6-7 MC4R+/− 42 week old male C57BL/6J mice per condition (FIG. 8) were implanted with Alzet minipump model #1002 subcutaneously in the subscapular region. Pumps were loaded prior to implantation so as to deliver an estimated 1200 nmol/kg/day. Cumulative food intake was monitored during low-dose infusion of peptide over two weeks (FIG. 8). Data points indicate mean±SEM.

SEQUENCES

SEQ ID NO: 1-X-AA1-AA1B-AA2-AA3-AA4-AA5-AA6-AA7-AA8-AA9-AA10-AA11-Y
SEQ ID NO: 2-Arg-Cys-D-Ala-His-D-Phe-Arg-Trp-Cys
SEQ ID NO: 3-D-Ala-His-D-Phe-Arg-Trp-Cys
SEQ ID NO: 4-D-Ala-Nme-His-D-Phe-Arg-Nme-Trp-Cys
SEQ ID NO: 5-Cys-Glu-His-D-Phe-Arg-Trp-Cys
SEQ ID NO: 6-Cys-Gly-His-D-Phe-Arg-Trp-Cys
SEQ ID NO: 7-Cys-D-Ala-His-D-Phe-Arg-Trp-Cys
SEQ ID NO: 8-Arg-Cys-D-Ala-Arg-D-Phe-Arg-Trp-Cys
SEQ ID NO: 9-Arg-Cys-D-Ala-His-Phe-Arg-Trp-Cys
SEQ ID NO: 10-Arg-Nle-Cys-His-D-Phe-Arg-Trp-Cys
SEQ ID NO: 11-Arg-Nle-Cys-Arg-D-Phe-Arg-Trp-Cys
SEQ ID NO: 12-Arg-Cys-D-Ala-Arg-D-Phe-Arg-D-Phe-Cys
SEQ ID NO: 13-Arg-Cys-D-Ala-His-D-Phe-Arg-D-Trp-Cys
SEQ ID NO: 14-Arg-Cys-D-Ala-Arg-D-Phe-Arg-D-Trp-Cys
SEQ ID NO: 15-Arg-Cys-D-Ala-Arg-D-Phe-Arg-D-Nal(2′)-Cys
SEQ ID NO: 16-Arg-Cys-D-Ala-Arg-D-Phe-Arg-D-Phe-Cys
SEQ ID NO: 17-Arg-Cys-Ala-Arg-D-Phe-Arg-Trp-Cys
SEQ ID NO: 18-Arg-Cys-Ala-Arg-D-Phe-Arg-D-Trp-Cys
SEQ ID NO: 19-Arg-Cys-NMe-Ala-Arg-D-Phe-Arg-D-Trp-Cys
SEQ ID NO: 20-Arg-Cys-Aib-Arg-D-Phe-Cit-Trp-Cys
SEQ ID NO: 21-Arg-Cys-D-Ala-Arg-aMe-D-Phe-Arg-Trp-Cys
SEQ ID NO: 22-Arg-Cys-D-Ala-Arg-D-Phe-Cit-D-Trp-Cys
SEQ ID NO: 23-Arg-Cys-D-Ala-Arg-D-Phe-N-Me-Arg-D-Trp-Cys
SEQ ID NO: 24-Arg-Cys-D-Ala-Cit-D-Phe-Arg-Trp-Cys
SEQ ID NO: 25-Arg-Cys-D-Ala-Cit-D-Phe-Arg-D-Trp-Cys
SEQ ID NO: 26-Arg-Cys-Ala-Cit-D-Phe-Arg-Trp-Cys
SEQ ID NO: 27-Arg-Cys-Ala-Cit-D-Phe-Arg-D-Trp-Cys
SEQ ID NO: 28-Arg-Cys-Aib-Cit-D-Phe-Arg-Trp-Cys
SEQ ID NO: 29-Arg-Cys-Aib-Cit-D-Phe-Arg-D-Trp-Cys
SEQ ID NO: 30-Arg-Cys-Aib-His-D-Phe-Arg-Trp-Cys
SEQ ID NO: 31-Arg-Cys-Aib-Arg-D-Phe-Arg-Trp-Cys
SEQ ID NO: 32-Arg-Cys-Gly-Arg-D-Phe-Arg-Trp-Cys
SEQ ID NO: 33-Arg-Cys-Gly-Cit-D-Phe-Arg-Trp-Cys
SEQ ID NO: 34-Arg-Cys-D-Abu-His-D-Phe-Arg-Trp-Cys
SEQ ID NO: 35-Arg-Cys-Aib-His-D-Phe-Arg-D-Trp-Cys
SEQ ID NO: 36-Arg-Cys-Aib-Arg-D-Tyr(4-OMe)-Arg-D-Trp-Cys
SEQ ID NO: 37-Arg-Cys-Aib-Arg-D-Tyr(4-OMe)-Arg-Phe-Cys
SEQ ID NO: 38-Arg-Cys-Aib-Cit-D-Tyr(4-OMe)-Arg-Phe-Cys
SEQ ID NO: 39-Arg-Cys-Aib-Arg-D-Phe-Arg-Phe-Cys
SEQ ID NO: 40-Arg-Cys-Aib-Orn-D-Phe-Arg-Phe-Cys
SEQ ID NO: 41-Arg-Cys-Aib-homo-Cit-D-Phe-Arg-Phe-Cys
SEQ ID NO: 42-Arg-Cys-Aib-Pal(2′)-D-Phe-Arg-Phe-Cys
SEQ ID NO: 43-Arg-Cys-Aib-Pal(3′)-D-Phe-Arg-Phe-Cys
SEQ ID NO: 44-Arg-Cys-Aib-Pal(4′)-D-Phe-Arg-Phe-Cys
SEQ ID NO: 45-Arg-Nle-Cys-Cit-D-Phe-Arg-Trp-Cys
SEQ ID NO: 46-Arg-Nle-Cys-Cit-D-Phe-Arg-D-Trp-Cys
SEQ ID NO: 47-Arg-Nle-Cys-Cit-Phe-Arg-Trp-Cys
SEQ ID NO: 48-Arg-Nle-Cys-His-Nal(1′)-Arg-Trp-Cys
SEQ ID NO: 49-Arg-Nle-Cys-Arg-D-Nal(1′)-Arg-Trp-Cys
SEQ ID NO: 50-Arg-Nle-Cys-Arg-D-Phe-Arg-D-Trp-Cys
SEQ ID NO: 51-Arg-Nle-Cys-Orn-D-Phe-Arg-Trp-Cys
SEQ ID NO: 52-Arg-Nle-Cys-homo-Cit-D-Phe-Arg-Trp-Cys
SEQ ID NO: 53-Arg-Nle-Cys-Pal(2′)-D-Phe-Arg-Trp-Cys
SEQ ID NO: 54-Arg-Nle-Cys-Pal(3′)-D-Phe-Arg-Trp-Cys
SEQ ID NO: 55-Arg-Nle-Cys-Pal(4′)-D-Phe-Arg-Trp-Cys
SEQ ID NO: 56-Arg-Nle-Cys-Arg-D-Phe-Arg-Phe-Cys
SEQ ID NO: 57-Arg-Nle-Cys-Arg-D-Phe(4-Br)-Arg-Phe-Cys
SEQ ID NO: 58-Arg-Nle-Cys-Arg-D-Tyr(4-OMe)-Arg-Phe-Cys
SEQ ID NO: 59-Arg-Nle-Cys-His-D-Phe-Arg-Phe-Cys
SEQ ID NO: 60-Arg-Nle-Cys-Cit-D-Phe-Arg-Phe-Cys
SEQ ID NO: 61-Cys-D-Ala-Arg-D-Phe-Arg-Trp-Cys
SEQ ID NO: 62-Cys-D-Ala-Cit-D-Phe-Arg-Trp-Cys
SEQ ID NO: 63-Cys-Gly-Arg-D-Phe-Arg-Trp-Cys
SEQ ID NO: 64-Cys-Gly-Cit-D-Phe-Arg-Trp-Cys
SEQ ID NO: 65-Cys-Aib-His-D-Phe-Arg-Trp-Cys
SEQ ID NO: 66-Cys-Aib-Arg-D-Phe-Arg-Trp-Cys
SEQ ID NO: 67-Cys-Aib-Cit-D-Phe-Arg-Trp-Cys
SEQ ID NO: 68-Cys-Aib-His-D-Phe-Arg-Phe-Cys
SEQ ID NO: 69-Cys-Aib-Arg-D-Phe-Arg-Phe-Cys
SEQ ID NO: 70-Cys-Aib-Cit-D-Phe-Arg-Phe-Cys
SEQ ID NO: 71-Cys-Gly-His-D-Phe-Arg-Phe-Cys
SEQ ID NO: 72-Cys-Gly-Arg-D-Phe-Arg-Phe-Cys
SEQ ID NO: 73-Cys-Gly-Cit-D-Phe-Arg-Phe-Cys
SEQ ID NO: 74-D-Ala-Arg-D-Phe-Arg-Trp-Cys
SEQ ID NO: 75-D-Ala-Cit-D-Phe-Arg-Trp-Cys
SEQ ID NO: 76-Gly-His-D-Phe-Arg-Trp-Cys
SEQ ID NO: 77-Gly-Arg-D-Phe-Arg-Trp-Cys
SEQ ID NO: 78-Gly-Cit-D-Phe-Arg-Trp-Cys
SEQ ID NO: 79-Aib-His-D-Phe-Arg-Trp-Cys
SEQ ID NO: 80-Aib-Arg-D-Phe-Arg-Trp-Cys
SEQ ID NO: 81-Aib-Cit-D-Phe-Arg-Trp-Cys
SEQ ID NO: 82-Aib-His-D-Phe-Arg-Phe-Cys
SEQ ID NO: 83-Aib-Arg-D-Phe-Arg-Phe-Cys
SEQ ID NO: 84-Aib-Cit-D-Phe-Arg-Phe-Cys
SEQ ID NO: 85-Gly-His-D-Phe-Arg-Phe-Cys
SEQ ID NO: 86-Gly-Arg-D-Phe-Arg-Phe-Cys
SEQ ID NO: 87-Gly-Cit-D-Phe-Arg-Phe-Cys
SEQ ID NO: 88-D-Ala-His-D-Phe-Arg-Phe-Cys
SEQ ID NO: 89-D-Ala-Arg-D-Phe-Arg-Phe-Cys
SEQ ID NO: 90-D-Ala-Cit-D-Phe-Arg-Phe-Cys
SEQ ID NO: 91-Nle-Cys-His-Nal(2′)-Arg-D-Trp-Cys-Arg-Phe-Gly
SEQ ID NO: 92-Arg-Cys-D-Ala-Arg-Phe(4-Br)-Arg-Trp-Cys
SEQ ID NO: 93-Arg-Cys-D-Ala-Arg-D-Phe(4-Br)-Arg-D-Trp-Cys
SEQ ID NO: 94-Arg-Cys-D-Ala-Arg-Phe(4-Br)-Arg-D-Trp-Cys
SEQ ID NO: 95-Arg-Cys-D-Ala-Arg-D-Phe(4-Br)-Arg-D-Trp-Cys-Lys-Pro-Val
SEQ ID NO: 96-Arg-Cys-D-Ala-Arg-D-Phe(4-Br)-Arg-D-Trp-Cys-Lys-Pro
SEQ ID NO: 97-Arg-Cys-D-Ala-Arg-D-Phe(4-Br)-Arg-D-Trp-Cys-Lys
SEQ ID NO: 98-Arg-Cys-D-Ala-Arg-D-Phe(4-Br)-Arg-D-Trp-Cys-Arg-Phe-Gly
SEQ ID NO: 99-Arg-Cys-D-Ala-Arg-D-Phe(4-Br)-Arg-D-Trp-Cys-Arg-Phe
SEQ ID NO: 100-Arg-Cys-D-Ala-Arg-D-Phe(4-Br)-Arg-D-Trp-Cys-Arg-D-Phe
SEQ ID NO: 101-Arg-Cys-D-Ala-Arg-D-Phe(4-Br)-Arg-D-Trp-Cys
SEQ ID NO: 102-Arg-Nle-Cys-His-D-Phe(4-Br)-Arg-Trp-Cys
SEQ ID NO: 103-Arg-Nle-Cys-Arg-D-Phe(4-Br)-Arg-Trp-Cys
SEQ ID NO: 104-Arg-Nle-Cys-His-D-Phe(4-Br)-Arg-D-Trp-Cys
SEQ ID NO: 105-Arg-Nle-Cys-Arg-D-Phe(4-Br)-Arg-D-Trp-Cys
SEQ ID NO: 106-Arg-Nle-Cys-His-D-Phe(4-1)-Arg-D-Trp-Cys
SEQ ID NO: 107-Nle-Cys-His-D-Phe(4-I)-Arg-D-Trp-Cys
SEQ ID NO: 108-Arg-Cys-D-Ala-Arg-D-Phe(4-F)-Arg-D-Trp-Cys
SEQ ID NO: 109-Arg-Cys-D-Ala-Arg-D-Tyr-Arg-D-Trp-Cys
SEQ ID NO: 110-Arg-Cys-D-Ala-Arg-D-Tyr(O-Me)-Arg-D-Trp-Cys
SEQ ID NO: 111-Arg-Cys-D-Ala-Arg-D-Hph-Arg-D-Trp-Cys
SEQ ID NO: 112-Arg-Cys-D-Ala-Arg-aMe-D-Phe-Arg-D-Trp-Cys
SEQ ID NO: 113-Arg-Cys-D-Ala-Arg-D-Phe(4-tBu)-Arg-D-Trp-Cys
SEQ ID NO: 114-Arg-Cys-D-Ala-Arg-D-Bip-Arg-D-Trp-Cys
SEQ ID NO: 115-Arg-Cys-D-Ala-Arg-D-Trp-Arg-D-Trp-Cys
SEQ ID NO: 116-Arg-Cys-D-Ala-Arg-D-Phe(4-NH-Ac)-Arg-D-Trp-Cys
SEQ ID NO: 117-Arg-Cys-Aib-Arg-D-Phe(4-Br)-Arg-D-Trp-Cys
SEQ ID NO: 118-Arg-Cys-D-Abu-Arg-D-Phe(4-Br)-Arg-D-Trp-Cys
SEQ ID NO: 119-Arg-Cys-D-Ala-NMe-Arg-D-Phe(4-Br)-Arg-D-Trp-Cys
SEQ ID NO: 120-Arg-Cys-D-Ala-Pro-D-Phe(4-Br)-Arg-D-Trp-Cys
SEQ ID NO: 121-Arg-Cys-D-Ala-D-Arg-D-Phe(4-Br)-Arg-D-Trp-Cys
SEQ ID NO: 122-Arg-Cys-D-Ala-Arg-D-Phe(4-Br)-NMe-Arg-D-Trp-Cys
SEQ ID NO: 123-Arg-Cys-D-Ala-Arg-D-Phe(4-Br)-D-Arg-D-Trp-Cys
SEQ ID NO: 124-Arg-Cys-D-Ala-Arg-D-Phe(4-Br)-Cit-D-Trp-Cys
SEQ ID NO: 125-Arg-Cys-D-Ala-Arg-D-Phe(4-Br)-Arg-D-Nal(2′)-Cys
SEQ ID NO: 126-Arg-Cys-D-Ala-Arg-D-Phe(4-Br)-Arg-D-Phe-Cys
SEQ ID NO: 127-Arg-Cys-D-Ala-Arg-D-Phe(4-Br)-Arg-D-Tic-Cys
SEQ ID NO: 128-Arg-Cys-D-Ala-Arg-D-Phe(4-Br)-Arg-Phe-Cys
SEQ ID NO: 129-Arg-Cys-D-Ala-Arg-D-Phe(4-Br)-Arg-Tic-Cys
SEQ ID NO: 130-Arg-Cys-D-Ala-Arg-D-Phe(4-Br)-Arg-D-Ala-Cys
SEQ ID NO: 131-Arg-Cys-Aib-Arg-D-Phe-Cit-D-Trp-Cys
SEQ ID NO: 132-Arg-Cys-D-Ala-Arg-NMe-D-Phe-Arg-D-Trp-Cys
SEQ ID NO: 133-Arg-Cys-D-Ala-Arg-D-Tic-Arg-D-Trp-Cys
SEQ ID NO: 134-Arg-Cys-D-Ala-Arg-D-Trp-Arg-Trp-Cys
SEQ ID NO: 135-Arg-Cys-D-Ala-Arg-D-Trp-Arg-D-Trp-Cys
SEQ ID NO: 136-Arg-Cys-D-Ala-Arg-D-Hph-Arg-D-Trp-Cys
SEQ ID NO: 137-Arg-Cys-D-Ala-Arg-a-Me-D-Phe-Arg-D-Trp-Cys
SEQ ID NO: 138-Arg-Cys-D-Ala-Arg-D-Dip-Arg-D-Trp-Cys
SEQ ID NO: 139-Arg-Cys-D-Ala-Arg-D-Phe-D-Arg-Trp-Cys
SEQ ID NO: 140-Arg-Cys-D-Ala-Arg-D-Phe-D-Arg-D-Trp-Cys
SEQ ID NO: 141-Arg-Cys-Aib-Cit-D-Tyr(4-OMe)-Arg-D-Trp-Cys
SEQ ID NO: 142-Arg-Nle-Cys-Arg-D-Phe(4-Br)-Arg-Trp-Cys
SEQ ID NO: 143-Arg-Nle-Cys-Arg-D-Phe(4-Br)-Arg-D-Trp-Cys
SEQ ID NO: 144-Arg-Nle-Cys-Arg-D-Tyr(4-OMe)-Arg-Trp-Cys
SEQ ID NO: 145-Arg-Nle-Cys-Arg-D-Tyr(4-OMe)-Arg-D-Trp-Cys
SEQ ID NO: 146-Cys-Gly-Arg-D-Phe(4-Br)-Arg-Phe-Cys
SEQ ID NO: 147-Cys-Gly-Arg-D-Tyr(4-OMe)-Arg-Phe-Cys
SEQ ID NO: 148-Arg-Cys-D-Ala-Pro-D-Nal(2′)-Arg-Trp-Cys
SEQ ID NO: 149-Arg-Cys-D-Ala-Pro-D-Phe(4-Br)-Arg-Trp-Cys
SEQ ID NO: 150-Arg-Cys-D-Ala-Arg-D-Phe(4-Br)-Arg-Trp-Cys
SEQ ID NO: 151-Arg-Cys-D-Ala-His-D-Phe(4-Br)-Arg-Trp-Cys

### REFERENCES

The following references are herein incorporated by reference in their entireties.
1. Andermann, M. L. & Lowell, B. B. Toward a Wiring Diagram Understanding of Appetite Control. Neuron (2017). doi:10.1016/j.neuron.2017.06.014
2. Sternson, S. M., Nicholas Betley, J. & Cao, Z. F. H. Neural circuits and motivational processes for hunger. Current Opinion in Neurobiology (2013). doi:10.1016/j.conb.2013.04.006
3. Sohn, J. W., Elmquist, J. K. & Williams, K. W. Neuronal circuits that regulate feeding behavior and metabolism. Trends in Neurosciences (2013). doi:10.1016/j.tins.2013.05.003
4. Mercer, A. J., Hentges, S. T., Meshul, C. K. & Low, M. J. Unraveling the central proopiomelanocortin neural circuits. Front. Neurosci. (2013). doi:10.3389/fnins.2013.00019
5. Cone, R. D. Anatomy and regulation of the central melanocortin system. Nature Neuroscience (2005). doi:10.1038/nn1455
6. Sternson, S. M. & Atasoy, D. Agouti-related protein neuron circuits that regulate appetite. Neuroendocrinology (2014). doi:10.1159/000369072
7. Krashes, M. J., Shah, B. P., Koda, S. & Lowell, B. B. Rapid versus delayed stimulation of feeding by the endogenously released agRP neuron mediators GABA, NPY, and AgRP. Cell Metab. (2013). doi:10.1016/j.cmet.2013.09.009
8. Zhan, C. et al. Acute and Long-Term Suppression of Feeding Behavior by POMC Neurons in the Brainstem and Hypothalamus, Respectively. J. Neurosci. (2013). doi:10.1523/jneurosci.2742-12.2013
9. Betley, J. N., Cao, Z. F. H., Ritola, K. D. & Sternson, S. M. Parallel, redundant circuit organization for homeostatic control of feeding behavior. Cell (2013). doi:10.1016/j.cell.2013.11.002
10. Atasoy, D., Nicholas Betley, J., Su, H. H. & Sternson, S. M. Deconstruction of a neural circuit for hunger. Nature (2012). doi:10.1038/nature11270
11. Luquet, S., Perez, F. A., Hnasko, T. S. & Palmiter, R. D. NPY/AgRP neurons are essentials for feeding in adult mice but can be ablated in neonates. Science (80- [symbol] ). (2005). doi:10.1126/science.1115524
12. Krashes, M. J. et al. Rapid, reversible activation of AgRP neurons drives feeding behavior in mice. J. Clin. Invest. (2011). doi:10.1172/JCI46229
13. Aponte, Y., Atasoy, D. & Sternson, S. M. AGRP neurons are sufficient to orchestrate feeding behavior rapidly and without training. Nat. Neurosci. (2011). doi:10.1038/nn.2739
14. Burnett, C. J. et al. Hunger-Driven Motivational State Competition. Neuron (2016). doi:10.1016/j.neuron.2016.08.032
15. Padilla, S. L. et al. Agouti-related peptide neural circuits mediate adaptive behaviors in the starved state. Nat. Neurosci. (2016). doi:10.1038/nn.4274
16. Bagnol, D. et al. Anatomy of an endogenous antagonist; relationship between Agouti-related protein and proopiomelanocortin in brain. J. Neurosci. (1999).
17. Roselli-Rehfuss, L. et al. Identification of a receptor for gamma melanotropin and other proopiomelanocortin peptides in the hypothalamus and limbic system. Proc. Natl. Acad. Sci. (2006). doi:10.1073/pnas.90.19.8856
18. Ghamari-Langroudi, M. et al. Regulation of energy rheostasis by the melanocortin-3 receptor. Sci. Adv. (2018). doi:10.1126/sciadv.aat0866
19. Sutton, G. M. et al. The Melanocortin-3 Receptor Is Required for Entrainment to Meal Intake. J. Neurosci. (2008). doi:10.1523/JNEUROSCI.3615-08.2008
20. Renquist, B. J. et al. Melanocortin-3 receptor regulates the normal fasting response. Proc. Natl. Acad. Sci. (2012). doi:10.1073/pnas.1201994109
21. Fenselau, H. et al. A rapidly acting glutamatergic ARC→PVH satiety circuit postsynaptically regulated by α-MSH. Nat. Neurosci. (2017). doi:10.1038/nn.4442
22. Armbruster, B. N., Li, X., Pausch, M. H., Herlitze, S. & Roth, B. L. Evolving the lock to fit the key to create a family of G protein-coupled receptors potently activated by an inert ligand. Proc. Natl. Acad. Sci. (2007). doi:10.1073/pnas.0700293104
23. Alexander, G. M. et al. Remote Control of Neuronal Activity in Transgenic Mice Expressing Evolved G Protein-Coupled Receptors. Neuron (2009). doi:10.1016/j.neuron.2009.06.014
24. Stachniak, T. J., Ghosh, A. & Sternson, S. M. Chemogenetic Synaptic Silencing of Neural Circuits Localizes a Hypothalamus→Midbrain Pathway for Feeding Behavior. Neuron (2014). doi:10.1016/j.neuron.2014.04.008
25. Li, C. et al. AGRP neurons modulate fasting-induced anxiolytic effects. Transl. Psychiatry (2019). doi:10.1038/s41398-019-0438-1
26. Dietrich, M. O., Zimmer, M. R., Bober, J. & Horvath, T. L. Hypothalamic Agrp neurons drive stereotypic behaviors beyond feeding. Cell (2015). doi:10.1016/j.cell.2015.02.024
27. Zelikowsky, M. et al. The Neuropeptide Tac2 Controls a Distributed Brain State Induced by Chronic Social Isolation Stress. Cell (2018). doi:10.1016/j.cell.2018.03.037
28. Ieraci, A., Mallei, A. & Popoli, M. Social Isolation Stress Induces Anxious-Depressive-Like Behavior and Alterations of Neuroplasticity-Related Genes in Adult Male Mice. Neural Plast. (2016). doi:10.1155/2016/6212983
29. Buynitsky, T. & Mostofsky, D. I. Restraint stress in biobehavioral research; Recent developments. Neuroscience and Biobehavioral Reviews (2009). doi:10.1016/j.neubiorev.2009.05.004
30. Campos, A. C., Fogaçca, M. V., Aguiar, D. C. & Guimarães, F. S. Animal models of anxiety disorders and stress. Rev. Bras. Psiquiatr. (2013). doi:10.1590/1516-4446-2013-1139
31. Grieco, P., Balse, P. M., Weinberg, D., MacNeil, T. & Hruby, V. J. D-amino acid scan of γ-melanocyte-stimulating hormone; Importance of Trp8 on human MC3 receptor selectivity. J. Med. Chem. (2000). doi:10.1021/jm000211e
32. Merlino, F. et al. Development of Macrocyclic Peptidomimetics Containing Constrained α,α-Dialkylated Amino Acids with Potent and Selective Activity at Human Melanocortin Receptors. J. Med. Chem. (2018). doi:10.1021/acs.jmedchem.8b00488
33. Carotenuto, A. et al. Discovery of Novel Potent and Selective Agonists at the Melanocortin-3 Receptor. J. Med. Chem. (2015). doi:10.1021/acs.jmedchem.5b01285
34. Herpertz-dahlmann, B., Holtkamp, K. & Konrad, K. Eating disorders; Anorexia and bulimia nervosa. Handb. Clin. Neurol. (2012). doi:10.1016/B978-0-444-52002-9.00026-7
35. Smink, F. R. E., Van Hoeken, D. & Hoek, H. W. Epidemiology, course, and outcome of eating disorders. Current Opinion in Psychiatry (2013). doi:10.1097/YCO.0b013e328365a24f
36. Jerlhag, E. et al. Ghrelin stimulates locomotor activity and accumbal dopamine-overflow via central cholinergic systems in mice; Implications for its involvement in brain reward. Addict. Biol. (2006). doi:10.1111/j.1369-1600.2006.00002.x
37. Adermark, L. et al. Ghrelin administration into tegmental areas stimulates locomotor activity and increases extracellular concentration of dopamine in the nucleus accumbens. Addict. Biol. (2007). doi:10.1111/j.1369-1600.2006.00041.x
38. Marks, D. L., Hruby, V., Brookhart, G. & Cone, R. D. The regulation of food intake by selective stimulation of the type 3 melanocortin receptor (MC3R). Peptides (2006). doi:10.1016/j.peptides.2005.01.025
39. Lee, M. et al. Effects of selective modulation of the central melanocortin-3-receptor on food intake and hypothalamic POMC expression. Peptides (2008). doi:10.1016/j.peptides.2007.11.005
40. Lippert, R. N., Ellacott, K. L. J. & Cone, R. D. Gender-specific roles for the melanocortin-3 receptor in the regulation of the mesolimbic dopamine system in mice. Endocrinology (2014). doi:10.1210/en.2013-2049
41. Pandit, R. et al. Melanocortin 3 receptor signaling in midbrain dopamine neurons increases the motivation for food reward. Neuropsychopharmacology (2016). doi:10.1038/npp.2016.19
42. Mavrikaki, M. et al. Melanocortin-3 receptors in the limbic system mediate feeding-related motivational responses during weight loss. Mol. Metab. (2016). doi:10.1016/j.molmet.2016.05.002
43. Pei, H. et al. Lateral Hypothalamic Mc3R-Expressing Neurons Modulate Locomotor Activity, Energy Expenditure, and Adiposity in Male Mice. Endocrinology 160, 343-358 (2018).
44. Sternson, S. M. & Eiselt, A.-K. Three Pillars for the Neural Control of Appetite. Annu. Rev. Physiol. (2016). doi:10.1146/annurev-physiol-021115-104948
45. Rossi, M. A. & Stuber, G. D. Overlapping Brain Circuits for Homeostatic and Hedonic Feeding. Cell Metabolism (2018). doi:10.1016/j.cmet.2017.09.021
46. Sweeney, P. & Yang, Y. Neural Circuit Mechanisms Underlying Emotional Regulation of Homeostatic Feeding. Trends in Endocrinology and Metabolism (2017). doi:10.1016/j.tem.2017.02.006
47. Hay, P. J., Touyz, S. & Sud, R. Treatment for severe and enduring anorexia nervosa; A review; Australian and New Zealand Journal of Psychiatry (2012). doi:10.1177/0004867412450469
48. Bulik, C. M. et al. Prevalence, heritability, and prospective risk factors for anorexia nervosa. Arch. Gen. Psychiatry (2006). doi:10.1001/archpsyc.63.3.305
49. Keski-Rahkonen, A. et al. Epidemiology and course of anorexia nervosa in the community. Am. J. Psychiatry (2007). doi:10.1176/appi.ajp.2007.06081388
50. Marks, D. L., Butler, A. A., Turner, R., Brookhart, G. & Cone, R. D. Differential role of melanocortin receptor subtypes in cachexia. Endocrinology (2003). doi:10.1210/en.2002-221099
51. Fazeli, P. K. et al. Treatment with a ghrelin agonist in outpatient women with anorexia nervosa; A randomized clinical trial. J. Clin. Psychiatry (2018). doi:10.4088/JCP.17m11585
52. Fazeli, P. et al. Short-term treatment with a ghrelin agonist significantly improves gastric emptying in anorexia nervosa. Endocrine reviews. Conference: 98th annual meeting and expo of the endocrine society, ENDO 2016. United states. Conference start: 20160401. Conference end; 20160404 (2016). doi:10.1210/endo-meetings.2016.OABA.2.SUN-606
53. Carlini, V. P. et al. Ghrelin increases anxiety-like behavior and memory retention in rats. Biochem. Biophys. Res. Commun. (2002).
54. Carvajal, P., Carlini, V. P., Schiöth, H. B., de Barioglio, S. R. & Salvatierra, N. A. Central ghrelin increases anxiety in the Open Field test and impairs retention memory in a passive avoidance task in neonatal chicks. Neurobiol. Learn. Mem. (2009). doi:10.1016/j.nlm.2008.12.008
55. Sweeney, P. & Yang, Y. An excitatory ventral hippocampus to lateral septum circuit that suppresses feeding. Nat. Commun. (2015). doi:10.1038/ncomms10188
56. Sweeney, P. & Yang, Y. An Inhibitory Septum to Lateral Hypothalamus Circuit That Suppresses Feeding. J. Neurosci. (2016). doi:10.1523/jneurosci.2042-16.2016
57. Sweeney, P., Li, C. & Yang, Y. Appetite suppressive role of medial septal glutamatergic neurons. Proc. Natl. Acad. Sci. (2017). doi:10.1073/pnas.1707228114
58. Singh, A., Dirain, M., Witek, R., Rocca J. R., Edison, A. S., and Haskell-Luevano, C. Structure-Activity Relationships of Peptides Incorporating a Bioactive Reverse-Turn Heterocycle at the Melanocortin Receptors; Identification of a 5800-fold Mouse Melanocortin-3 Receptor (mMC3R) Selective Antagonist/Partial Agonist versus the Mouse Melanocortin-4 Receptor (mMC4R). J. Med. Chem, 56, 2747-2763, 2013.

1. A composition comprising a peptide having 4 or fewer substitutions relative to the sequence;
(SEQ ID NO: 1)
X-AA1-AA1B-AA2-AA3-AA4-AA5-AA6-AA7-AA8-AA9-AA10-
AA11-Y;
wherein X is a N-terminal cap moiety linked to the most N-terminal amino acid of the peptide and is acetyl, chloro acetyl, or absent;
wherein AA1 is Arg or absent;
wherein AA1B is Nle or absent;
wherein AA2 is Cys or absent;
wherein AA3 is D-Ala, Glu, D-Glu, D-Gly, D-Aib, Gly, Ala, NMe-Ala, Aib, Abu, D-Abu, or absent;
wherein AA4 is Arg, D-Arg, NMe-Arg, NMe-D-Arg, Cit, D-Cit, His, D-His, Nme-His, NMe-D-His, Pro, D-Orn, Orn, Homo-Arg, Homo-Cit, Homo-D-Cit, Pal(2′), Pal(3′), Pal(4′), D-Pal(2′), D-Pal(3), 4-guanidyl-Dab, 4-guanidyl-D-Dab, 3-guanidyl-Dap, 3-guanidyl-D-Dap, 5-carbamoyl-Dab, 5-carbamoyl-D-Dab, 3-carbamoyl-Dap, 3-carbamoyl-D-Dap, or D-Pal(4′);
wherein AA5 is Phe, D-Phe, D-Phe(4-Br), D-Phe(4-I), D-Phe(4-F), D-Phe(4-tBu), Phe(4-Br), Phe(4-F), Nal(2′), D-Nal(2′), Nal(1′), D-Tyr, Tyr(4-OMe), or D-Tyr(4-OMe), D-Hph, D-Bip, D-Tic, D-Dip, D-Trp, aMe-D-Phe, D-Phe(4-NH-Ac), NMe-D-Phe, Phe(4-tBu), Trp, Hph, Bip, Tic, Dip, D-Nal(1′), aMe-Phe, Phe(4-NH-Ac), NMe-Phe, Tyr, D-Nal(1′), Phe(4-I), D-Phe(4-I), Phe(4-tBu)], D-Phe(4-guanidyl), or Phe(4-guanidyl);
wherein AA6 is Arg, NMe-Arg, D-Arg, Cit, NMe-D-Arg, or D-Cit;
wherein AA7 is Trp, D-Trp, NMe-Trp, Phe, D-Phe, D-Ala D-Nal(2′), D-Tic, NMe-D-Trp, Ala, Nal(2′), or Tic;
wherein AA8 is Cys;
wherein AA9 is Lys, Arg, or absent;
wherein AA10 is Pro, Phe, D-Phe, or absent;
wherein AA11 is Val, Gly, or absent;
wherein Y is a C-terminal cap linked to the most C-terminal amino acid of the peptide and is NH₂ or absent;
wherein AA1 is linked to AA2 if AA1B is absent;
wherein AA9 is present if AA10 is present;
wherein AA9 and AA10 are present if AA11 is present;
wherein the peptide does not consist of Arg-Cys-(D-Ala)-His-(D-Phe)-Arg-Trp-Cys (SEQ ID NO: 2).

2. The composition of claim 1, wherein the peptide comprises 100% sequence similarity to the sequence;
(SEQ ID NO: 1)
X-AA1-AA1B-AA2-AA3-AA4-AA5-AA6-AA7-AA8-AA9-AA10-
AA11-Y;
wherein X is a N-terminal cap moiety linked to the most N-terminal amino acid of the peptide and is acetyl, chloro acetyl, or absent;
wherein AA1 is Arg or absent;
wherein AA1B is Nle or absent;
wherein AA2 is Cys or absent;
wherein AA3 is D-Ala, Glu, D-Glu, D-Gly, D-Aib, Gly, Ala, NMe-Ala, Aib, Abu, D-Abu, or absent;
wherein AA4 is Arg, D-Arg, NMe-Arg, NMe-D-Arg, Cit, D-Cit, His, D-His, Nme-His, NMe-D-His, Pro, D-Om, Orn, Homo-Arg, Homo-Cit, Homo-D-Cit, Pal(2′), Pal(3′), Pal(4′), D-Pal(2′), D-Pal(3′), 4-guanidyl-Dab, 4-guanidyl-D-Dab, 3-guanidyl-Dap, 3-guanidyl-D-Dap, 5-carbamoyl-Dab, 5-carbamoyl-D-Dab, 3-carbamoyl-Dap, 3-carbamoyl-D-Dap, or D-Pal(4′);
wherein AA5 is Phe, D-Phe, D-Phe(4-Br), D-Phe(4-I), D-Phe(4-F), D-Phe(4-tBu), Phe(4-Br), Phe(4-F), Nal(2′), D-Nal(2′), Nal(1′), D-Tyr, Tyr(4-OMe), or D-Tyr(4-OMe), D-Hph, D-Bip, D-Tic, D-Dip, D-Trp, aMe-D-Phe, D-Phe(4-NH-Ac), NMe-D-Phe, Phe(4-tBu), Trp, Hph, Bip, Tic, Dip, D-Nal(1′), aMe-Phe, Phe(4-NH-Ac), NMe-Phe, Tyr, D-Nal(1′), Phe(4-I), D-Phe(4-I), Phe(4-tBu)], D-Phe(4-guanidyl), or Phe(4-guanidyl);
wherein AA6 is Arg, NMe-Arg, D-Arg, Cit, NMe-D-Arg, or D-Cit;
wherein AA7 is Trp, D-Trp, NMe-Trp, Phe, D-Phe, D-Ala D-Nal(2′), D-Tic, NMe-D-Trp, Ala, Nal(2′), or Tic;
wherein AA8 is Cys;
wherein AA9 is Lys, Arg, or absent;
wherein AA10 is Pro, Phe, D-Phe, or absent;
wherein AA11 is Val, Gly, or absent;
wherein Y is a C-terminal cap linked to the most C-terminal amino acid of the peptide and is NH₂ or absent;
wherein AA1 is linked to AA2 if AA1B is absent;
wherein AA9 is present if AA10 is present;
wherein AA9 and AA10 are present if AA11 is present;
wherein the peptide does not consist of Arg-Cys-(D-Ala)-His-(D-Phe)-Arg-Trp-Cys (SEQ ID NO: 2).

3. The composition of claim 1, wherein the peptide comprises the sequence;
(SEQ ID NO: 1)
X-AA1-AA1B-AA2-AA3-AA4-AA5-AA6-AA7-AA8-AA9-AA10-
AA11-Y;
wherein X is a N-terminal cap moiety linked to the most N-terminal amino acid of the peptide and is acetyl, chloro acetyl, or absent;
wherein AA1 is Arg or absent;
wherein AA1B is Nle or absent;
wherein AA2 is Cys or absent;
wherein AA3 is D-Ala, Glu, D-Glu, D-Gly, D-Aib, Gly, Ala, NMe-Ala, Aib, Abu, D-Abu, or absent;
wherein AA4 is Arg, D-Arg, NMe-Arg, NMe-D-Arg, Cit, D-Cit, His, D-His, Nme-His, NMe-D-His, Pro, D-Om, Orn, Homo-Arg, Homo-Cit, Homo-D-Cit, Pal(2′), Pal(3′), Pal(4′), D-Pal(2′), D-Pal(3′), 4-guanidyl-Dab, 4-guanidyl-D-Dab, 3-guanidyl-Dap, 3-guanidyl-D-Dap, 5-carbamoyl-Dab, 5-carbamoyl-D-Dab, 3-carbamoyl-Dap, 3-carbamoyl-D-Dap, or D-Pal(4′);
wherein AA5 is Phe, D-Phe, D-Phe(4-Br), D-Phe(4-I), D-Phe(4-F), D-Phe(4-tBu), Phe(4-Br), Phe(4-F), Nal(2′), D-Nal(2′), Nal(1′), D-Tyr, Tyr(4-OMe), or D-Tyr(4-OMe), D-Hph, D-Bip, D-Tic, D-Dip, D-Trp, aMe-D-Phe, D-Phe(4-NH-Ac), NMe-D-Phe, Phe(4-tBu), Trp, Hph, Bip, Tic, Dip, D-Nal(1′), aMe-Phe, Phe(4-NH-Ac), NMe-Phe, Tyr, D-Nal(1′), Phe(4-I), D-Phe(4-I), Phe(4-tBu)], D-Phe(4-guanidyl), or Phe(4-guanidyl);
wherein AA6 is Arg, NMe-Arg, D-Arg, Cit, NMe-D-Arg, or D-Cit;
wherein AA7 is Trp, D-Trp, NMe-Trp, Phe, D-Phe, D-Ala D-Nal(2′), D-Tic, NMe-D-Trp, Ala, Nal(2′), or Tic;
wherein AA8 is Cys;
wherein AA9 is Lys, Arg, or absent;
wherein AA10 is Pro, Phe, D-Phe, or absent;
wherein AA11 is Val, Gly, or absent;
wherein Y is a C-terminal cap linked to the most C-terminal amino acid of the peptide and is NH₂ or absent;
wherein AA1 is linked to AA2 if AA1B is absent;
wherein AA9 is present if AA10 is present;
wherein AA9 and AA10 are present if AA11 is present;
wherein the peptide does not consist of Arg-Cys-(D-Ala)-His-(D-Phe)-Arg-Trp-Cys (SEQ ID NO: 2).

4. A composition comprising a peptide having 1-4 substitutions or terminal deletions relative to the amino acid sequence Arg-Cys-(D-Ala)-His-(D-Phe)-Arg-Trp-Cys (SEQ ID NO: 2).

5. The composition of one of claims 1-4, wherein the peptide is selected from one or SEQ ID NOS: 3-151.

6. The composition of one of claims 1-5, wherein the peptide comprises one or more non-proteinogenic amino acids or amino acid analogs.

7. The composition of one of claim 1-6, wherein AA3 is absent and AA1B is present.

8. The composition of one of claim 1-6, wherein AA3 is present and AA1B is absent.

9. The composition of one of claim 1-8, wherein all or a portion of the peptide is cyclic.

10. The composition of claim 9, wherein X is chloroacetyl, AA1, AA1b, and AA2 are absent, and the chloroacetyl reacts with the Cys at AA8 to form a thioether-linked cyclic peptide.

11. The composition of claim 10, wherein the peptide comprises an amino acid sequence selected from SEQ ID NOS: 3-4 and 74-90.

12. The composition of claim 9, wherein the amino acid corresponding to AA2 of SEQ ID NO: 1 is Cys, the amino acid corresponding to AA8 of SEQ ID NO: 1 is Cys, wherein the amino acid corresponding to AA2 of SEQ ID NO: 1 is linked to the amino acid corresponding to AA8 of SEQ ID NO: 1, and wherein the peptide segment corresponding to AA2-AA8 of SEQ ID NO: 1 is cyclic.

13. The composition of claim 9 or 12, wherein the peptide further comprises an amino acid corresponding to AA1 or AA1b of SEQ ID NO: 1 linked to the amino acid corresponding to AA2 of SEQ ID NO: 1 and/or an amino acid corresponding to AA9 of SEQ ID NO: 1 linked to the amino acid corresponding to AA8 of SEQ ID NO: 1.

14. The composition of claim 13, wherein the peptide comprises an amino acid sequence selected from SEQ ID NOS: 5-73 and 91-151.

15. The composition of one of claims 1-14, wherein the peptide a melanocortin 4 receptor (MC4R) agonist.

16. The composition of one of claims 1-14, wherein the peptide is a melanocortin 3 receptor (MC3R) antagonist.

17. The composition of one of claims 1-14, wherein the peptide is a melanocortin 3 receptor (MC3R) partial agonist.

18. A method of treating a subject for a disease, condition, or disorder comprising administering a composition of one of claims 1-17 to a subject.

19. The method of claim 18, wherein the subject suffers from positive energy balance as the cause or result of the disease, condition, or disorder

20. The method of claim 18, wherein the disease, condition, or disorder is characterized by overeating.

21. The method of claim 18, wherein the disease, condition, or disorder characterized by one or more emotional/mental symptoms.

22. The method of claim 18, wherein the disease, condition, or disorder is caused by or is the result of obesity.

23. The method of claim 18, wherein the subject suffers from diabetes, heart disease, hypertension, sleep apnea, depression, kidney disease, and/or arthritis.

24. The method of claim 23, wherein the composition is co-administered with nutritional therapy, psychotherapy, or other pharmaceutical agents.

25. The method of one of claims 18-24, wherein the administration is repeated on a recurring basis for a period of at least 1 week.

26. The method of claim 25, wherein the administration is repeated on a daily basis.

27. The method of claim 25, wherein the administration is repeated on a recurring basis for a period of at least 1 month.

28. The method of claim 27, wherein the administration is repeated on a recurring basis for a period of at least 1 year.

29. Use of a composition of one of claims 1-17 in the treatment or prevention of an condition, disease, or disorder.

30. Use of a composition of one of claims 1-17 as a medicament.

31. A composition of one of claims 1-17 for use in the manufacture of a medicament.
